SEQUENCE LISTING (247 sequences)

SEQ ID NO: 1 - DNA, 20 bp, synthetic construct
  source 1..20: mol_type = other DNA
tagcctggatctccttggtg

SEQ ID NO: 2 - DNA, 20 bp, synthetic construct
  source 1..20: mol_type = other DNA
gcggtacctgtagtgtgcag

SEQ ID NO: 3 - DNA, 25 bp, synthetic construct
  source 1..25: mol_type = other DNA
gcagagaaacaaatgaaggacaaac

SEQ ID NO: 4 - DNA, 20 bp, synthetic construct
  source 1..20: mol_type = other DNA
cttcctccttgtcctcctca

SEQ ID NO: 5 - DNA, 22 bp, synthetic construct
  source 1..22: mol_type = other DNA
tttctttgaagcagcaagtatg

SEQ ID NO: 6 - DNA, 22 bp, synthetic construct
  source 1..22: mol_type = other DNA
agatgctctgagaaaggcatta

SEQ ID NO: 7 - DNA, 23 bp, synthetic construct
  source 1..23: mol_type = other DNA
ccaacctcaccaacattacagag

SEQ ID NO: 8 - DNA, 26 bp, synthetic construct
  source 1..26: mol_type = other DNA
tccaatgtcacatgaatgtaaatcaa

SEQ ID NO: 9-20 - skipped (12 entries)

SEQ ID NO: 21 - DNA, 23 bp, synthetic construct
  source 1..23: mol_type = other DNA
ctcttgaagaatgaaagccttgg

SEQ ID NO: 22 - DNA, 25 bp, synthetic construct
  source 1..25: mol_type = other DNA
gtttctgtgtgctttatccagaact

SEQ ID NO: 23-25 - skipped (3 entries)

SEQ ID NO: 26 - DNA, 26 bp, synthetic construct
  source 1..26: mol_type = other DNA
tgcctattcctaactgactcatcatt

SEQ ID NO: 27 - DNA, 23 bp, synthetic construct
  source 1..23: mol_type = other DNA
gaaacatttggcacattccattc

SEQ ID NO: 28 - DNA, 23 bp, synthetic construct
  source 1..23: mol_type = other DNA
aatgcacgtactcaccatttgtc

SEQ ID NO: 29 - DNA, 22 bp, synthetic construct
  source 1..22: mol_type = other DNA
catctttgttgctgtccttcca

SEQ ID NO: 30 - DNA, 27 bp, synthetic construct
  source 1..27: mol_type = other DNA
ttacatttttaatgctcctttctttga

SEQ ID NO: 31 - DNA, 22 bp, synthetic construct
  source 1..22: mol_type = other DNA
gatgaggtgattttcgtggaag

SEQ ID NO: 32 - DNA, 20 bp, synthetic construct
  source 1..20: mol_type = other DNA
cttgtcacccacgggaaagt

SEQ ID NO: 33 - DNA, 26 bp, synthetic construct
  source 1..26: mol_type = other DNA
aaaataagtaggaaatagcagcctca

SEQ ID NO: 34 - DNA, 24 bp, synthetic construct
  source 1..24: mol_type = other DNA
tctctgtcctcacagagttcaagc

SEQ ID NO: 35 - DNA, 20 bp, synthetic construct
  source 1..20: mol_type = other DNA
cttggtccagccagggacta

SEQ ID NO: 36 - DNA, 21 bp, synthetic construct
  source 1..21: mol_type = other DNA
caaaaacatcccacgcctagt

SEQ ID NO: 37 - DNA, 21 bp, synthetic construct
  source 1..21: mol_type = other DNA
tggggtgaagaccattttgaa

SEQ ID NO: 38 - DNA, 23 bp, synthetic construct
  source 1..23: mol_type = other DNA
gagaagagggttgaggagttcaa

SEQ ID NO: 39 - DNA, 24 bp, synthetic construct
  source 1..24: mol_type = other DNA
gcaaaatcacattattgccaacat

SEQ ID NO: 40 - DNA, 22 bp, synthetic construct
  source 1..22: mol_type = other DNA
ctagtgcattcaagcacaatgg

SEQ ID NO: 41 - DNA, 23 bp, synthetic construct
  source 1..23: mol_type = other DNA
cagagcctaaacatccccttaaa

SEQ ID NO: 42 - DNA, 25 bp, synthetic construct
  source 1..25: mol_type = other DNA
aggtgatctatttttccctttctcc

SEQ ID NO: 43 - DNA, 24 bp, synthetic construct
  source 1..24: mol_type = other DNA
ttatgtgtacccaaaaaggtgaca

SEQ ID NO: 44 - DNA, 20 bp, synthetic construct
  source 1..20: mol_type = other DNA
tgccagttgtgctttttgct

SEQ ID NO: 45 - DNA, 23 bp, synthetic construct
  source 1..23: mol_type = other DNA
acaataaaaggcagcttggacac

SEQ ID NO: 46 - DNA, 23 bp, synthetic construct
  source 1..23: mol_type = other DNA
acttggcagccagaaatatcctc

SEQ ID NO: 47 - DNA, 25 bp, synthetic construct
  source 1..25: mol_type = other DNA
gactgtcaagcagagaatgggtact

SEQ ID NO: 48 - DNA, 25 bp, synthetic construct
  source 1..25: mol_type = other DNA
gatgtctatgaagtgttgtggttcc

SEQ ID NO: 49 - DNA, 27 bp, synthetic construct
  source 1..27: mol_type = other DNA
tgttacagaaatgaaataagacggaaa

SEQ ID NO: 50 - DNA, 20 bp, synthetic construct
  source 1..20: mol_type = other DNA
gaggctcgccatgccgggag

SEQ ID NO: 51 - DNA, 22 bp, synthetic construct
  source 1..22: mol_type = other DNA
gatgtcgatggacgtctcgtgc

SEQ ID NO: 52 - DNA, 20 bp, synthetic construct
  source 1..20: mol_type = other DNA
cagcgccgccttcggctttc

SEQ ID NO: 53 - DNA, 20 bp, synthetic construct
  source 1..20: mol_type = other DNA
gcccgggtagtcaaagtcgc

SEQ ID NO: 54 - DNA, 20 bp, synthetic construct
  source 1..20: mol_type = other DNA
ggcagcaggagaaggccaag

SEQ ID NO: 55 - DNA, 21 bp, synthetic construct
  source 1..21: mol_type = other DNA
ttcatcctcctcgcggggctc

SEQ ID NO: 56 - DNA, 19 bp, synthetic construct
  source 1..19: mol_type = other DNA
cggccgctggtgatcaagc

SEQ ID NO: 57 - DNA, 19 bp, synthetic construct
  source 1..19: mol_type = other DNA
ggtgaccgggctgcaggtg

SEQ ID NO: 58 - DNA, 19 bp, synthetic construct
  source 1..19: mol_type = other DNA
cgcactgcggccagaccac

SEQ ID NO: 59 - DNA, 22 bp, synthetic construct
  source 1..22: mol_type = other DNA
gttgctgttcttgtccaccgac

SEQ ID NO: 60 - DNA, 20 bp, synthetic construct
  source 1..20: mol_type = other DNA
gcgggcaaggccaagaagtc

SEQ ID NO: 61 - DNA, 20 bp, synthetic construct
  source 1..20: mol_type = other DNA
gtgtccagttcgcggctcag

SEQ ID NO: 62 - DNA, 22 bp, synthetic construct
  source 1..22: mol_type = other DNA
gaccagtgacaatgaccgcctg

SEQ ID NO: 63 - DNA, 19 bp, synthetic construct
  source 1..19: mol_type = other DNA
ggcgaccccaaaccactcc

SEQ ID NO: 64 - DNA, 23 bp, synthetic construct
  source 1..23: mol_type = other DNA
ccttcctcttactctctgcctgc

SEQ ID NO: 65 - DNA, 21 bp, synthetic construct
  source 1..21: mol_type = other DNA
gacagcgggcacacttaccag

SEQ ID NO: 66 - DNA, 22 bp, synthetic construct
  source 1..22: mol_type = other DNA
cattgttagggccgaggctaga

SEQ ID NO: 67 - DNA, 26 bp, synthetic construct
  source 1..26: mol_type = other DNA
acgcacttgttttacctgtatgagtc

SEQ ID NO: 68 - DNA, 27 bp, synthetic construct
  source 1..27: mol_type = other DNA
gcagattcaactttcacgtatcaaacc

SEQ ID NO: 69 - DNA, 22 bp, synthetic construct
  source 1..22: mol_type = other DNA
tgcagagcacgggctttaatgt

SEQ ID NO: 70 - DNA, 22 bp, synthetic construct
  source 1..22: mol_type = other DNA
cctcgcagacattaaagcccgt

SEQ ID NO: 71 - DNA, 21 bp, synthetic construct
  source 1..21: mol_type = other DNA
ccaccatcaccactgctgctg

SEQ ID NO: 72 - DNA, 21 bp, synthetic construct
  source 1..21: mol_type = other DNA
cagtggtgatggtggtgaggc

SEQ ID NO: 73 - DNA, 23 bp, synthetic construct
  source 1..23: mol_type = other DNA
cctagcccatctgtgagtccaac

SEQ ID NO: 74 - DNA, 22 bp, synthetic construct
  source 1..22: mol_type = other DNA
tgtcctcccaaacctcagtagc

SEQ ID NO: 75 - DNA, 22 bp, synthetic construct
  source 1..22: mol_type = other DNA
agcttggccagttcctttctct

SEQ ID NO: 76 - DNA, 22 bp, synthetic construct
  source 1..22: mol_type = other DNA
gtgtctcgagtatgtgcggtcc

SEQ ID NO: 77 - DNA, 21 bp, synthetic construct
  source 1..21: mol_type = other DNA
catcgtgggctggtggaagaa

SEQ ID NO: 78 - DNA, 22 bp, synthetic construct
  source 1..22: mol_type = other DNA
tccattgcctctccttctgtgc

SEQ ID NO: 79 - DNA, 21 bp, synthetic construct
  source 1..21: mol_type = other DNA
tgccaactccttcatgcacct

SEQ ID NO: 80 - DNA, 22 bp, synthetic construct
  source 1..22: mol_type = other DNA
cctaggggatgttccagatggc

SEQ ID NO: 81 - DNA, 24 bp, synthetic construct
  source 1..24: mol_type = other DNA
aatgtgggtttgttgccatgaaac

SEQ ID NO: 82 - DNA, 22 bp, synthetic construct
  source 1..22: mol_type = other DNA
tctatcgtgtccccacagggaa

SEQ ID NO: 83 - DNA, 22 bp, synthetic construct
  source 1..22: mol_type = other DNA
ggggaaaggttgaacccaagga

SEQ ID NO: 84 - DNA, 20 bp, synthetic construct
  source 1..20: mol_type = other DNA
cttcacgccgccttccaccg

SEQ ID NO: 85 - DNA, 21 bp, synthetic construct
  source 1..21: mol_type = other DNA
ctccgggccagtaccttgaaa

SEQ ID NO: 86 - DNA, 21 bp, synthetic construct
  source 1..21: mol_type = other DNA
tcctgctgtgtggttagacgg

SEQ ID NO: 87 - DNA, 21 bp, synthetic construct
  source 1..21: mol_type = other DNA
gaagaggtggcggatgactgg

SEQ ID NO: 88 - DNA, 23 bp, synthetic construct
  source 1..23: mol_type = other DNA
gccctgggcattccttctttatt

SEQ ID NO: 89 - DNA, 24 bp, synthetic construct
  source 1..24: mol_type = other DNA
acttctaagatgtggcaagatggc

SEQ ID NO: 90 - DNA, 25 bp, synthetic construct
  source 1..25: mol_type = other DNA
gttggggcatagttaaaacctgtgt

SEQ ID NO: 91 - DNA, 22 bp, synthetic construct
  source 1..22: mol_type = other DNA
ttcctctgtgttggcggatacc

SEQ ID NO: 92 - DNA, 22 bp, synthetic construct
  source 1..22: mol_type = other DNA
ggtatccgccaacacagaggaa

SEQ ID NO: 93 - DNA, 26 bp, synthetic construct
  source 1..26: mol_type = other DNA
aggagactggaattctcgaataagga

SEQ ID NO: 94-186 - skipped (93 entries)

SEQ ID NO: 187 - DNA, 19 bp, synthetic construct
  source 1..19: mol_type = other DNA
aggaaacagctatgaccat

SEQ ID NO: 188 - DNA, 18 bp, synthetic construct
  source 1..18: mol_type = other DNA
tgtaaaacgacggccagt

SEQ ID NO: 189 - skipped

SEQ ID NO: 190 - DNA, 10 bp, synthetic construct
  source 1..10: mol_type = other DNA
gcccccgccg

SEQ ID NO: 191 - DNA, 20 bp, synthetic construct
  source 1..20: mol_type = other DNA
gcggcccgccgcccccgccg

SEQ ID NO: 192 - DNA, 21 bp, synthetic construct
  source 1..21: mol_type = other DNA
ctttcacgtatcaaaccaccc

SEQ ID NO: 193 - DNA, 19 bp, synthetic construct
  source 1..19: mol_type = other DNA
cacgggctttaatgtctgc

SEQ ID NO: 194 - DNA, 21 bp, synthetic construct
  source 1..21: mol_type = other DNA
aagatcccagattccctactg

SEQ ID NO: 195 - DNA, 18 bp, synthetic construct
  source 1..18: mol_type = other DNA
gtgggctggtggaagaac

SEQ ID NO: 196 - DNA, 18 bp, synthetic construct
  source 1..18: mol_type = other DNA
ggcgagaggtcaccactg

SEQ ID NO: 197 - DNA, 18 bp, synthetic construct
  source 1..18: mol_type = other DNA
ccatcaccactgctgctg

SEQ ID NO: 198 - DNA, 20 bp, synthetic construct
  source 1..20: mol_type = other DNA
aactgaatgtgagtctggca

SEQ ID NO: 199 - DNA, 20 bp, synthetic construct
  source 1..20: mol_type = other DNA
cactagagacggaatgggac

SEQ ID NO: 200 - DNA, 19 bp, synthetic construct
  source 1..19: mol_type = other DNA
cagtggtgatggtggtgag

SEQ ID NO: 201 - DNA, 20 bp, synthetic construct
  source 1..20: mol_type = other DNA
ccaactgtagccctctgtag

SEQ ID NO: 202 - DNA, 19 bp, synthetic construct
  source 1..19: mol_type = other DNA
ctgctgtgtggttagacgg

SEQ ID NO: 203 - DNA, 18 bp, synthetic construct
  source 1..18: mol_type = other DNA
gaggtggcggatgactgg

SEQ ID NO: 204 - DNA, 18 bp, synthetic construct
  source 1..18: mol_type = other DNA
agcaacgcccatttcacc

SEQ ID NO: 205 - DNA, 20 bp, synthetic construct
  source 1..20: mol_type = other DNA
gctcagctgcaaagaatgtg

SEQ ID NO: 206 - DNA, 19 bp, synthetic construct
  source 1..19: mol_type = other DNA
caggcaagatgagcgaggc

SEQ ID NO: 207 - DNA, 20 bp, synthetic construct
  source 1..20: mol_type = other DNA
gggccagtaccttgaaagcg

SEQ ID NO: 208 - DNA, 21 bp, synthetic construct
  source 1..21: mol_type = other DNA
ggctgctggtctggctactat

SEQ ID NO: 209 - DNA, 19 bp, synthetic construct
  source 1..19: mol_type = other DNA
ccagggcagaggctacaac

SEQ ID NO: 210 - DNA, 20 bp, synthetic construct
  source 1..20: mol_type = other DNA
tgtaggtcttgtggatgagc

SEQ ID NO: 211 - DNA, 20 bp, synthetic construct
  source 1..20: mol_type = other DNA
ttcctctgtgttggcggata

SEQ ID NO: 212 - DNA, 22 bp, synthetic construct
  source 1..22: mol_type = other DNA
cagaggaaaggtaaatccacca

SEQ ID NO: 213 - DNA, 25 bp, synthetic construct
  source 1..25: mol_type = other DNA
ggaataagatacccaatagccttca

SEQ ID NO: 214 - DNA, 23 bp, synthetic construct
  source 1..23: mol_type = other DNA
ttccttctttattgcccttctta

SEQ ID NO: 215 - DNA, 26 bp, synthetic construct
  source 1..26: mol_type = other DNA
caacttaccatgttcaatgatttcaa

SEQ ID NO: 216 - DNA, 18 bp, synthetic construct
  source 1..18: mol_type = other DNA
ccacattgttagggccga

SEQ ID NO: 217 - DNA, 22 bp, synthetic construct
  source 1..22: mol_type = other DNA
cgcacttgttttacctgtatga

SEQ ID NO: 218 - DNA, 22 bp, synthetic construct
  source 1..22: mol_type = other DNA
gcctcccttcctcttactctct

SEQ ID NO: 219 - DNA, 19 bp, synthetic construct
  source 1..19: mol_type = other DNA
gcagcctgggtaagcacac

SEQ ID NO: 220 - DNA, 20 bp, synthetic construct
  source 1..20: mol_type = other DNA
ccaacgtcagtgaggcagat

SEQ ID NO: 221 - DNA, 21 bp, synthetic construct
  source 1..21: mol_type = other DNA
ggatgacaataccaggcagga

SEQ ID NO: 222 - DNA, 20 bp, synthetic construct
  source 1..20: mol_type = other DNA
tgctttccttggagccaaat

SEQ ID NO: 223 - DNA, 24 bp, synthetic construct
  source 1..24: mol_type = other DNA
tgtgttagctggagtgaaaacttg

SEQ ID NO: 224 - DNA, 22 bp, synthetic construct
  source 1..22: mol_type = other DNA
atatgcctgagcgtttcgatgc

SEQ ID NO: 225 - DNA, 21 bp, synthetic construct
  source 1..21: mol_type = other DNA
gtggccttctagccaacctct

SEQ ID NO: 226 - DNA, 21 bp, synthetic construct
  source 1..21: mol_type = other DNA
cccaggggatatgctgaacta

SEQ ID NO: 227 - DNA, 20 bp, synthetic construct
  source 1..20: mol_type = other DNA
acctggagagaggctgagtg

SEQ ID NO: 228 - DNA, 20 bp, synthetic construct
  source 1..20: mol_type = other DNA
ttggcttgcaggtgcccatc

SEQ ID NO: 229 - DNA, 22 bp, synthetic construct
  source 1..22: mol_type = other DNA
ggcgagtccagaaccaagattt

SEQ ID NO: 230 - DNA, 22 bp, synthetic construct
  source 1..22: mol_type = other DNA
ctactcctcgcctgtggacaac

SEQ ID NO: 231 - DNA, 22 bp, synthetic construct
  source 1..22: mol_type = other DNA
gatctgggactgcatgctggtg

SEQ ID NO: 232 - DNA, 22 bp, synthetic construct
  source 1..22: mol_type = other DNA
gtattcacagagacttggcagc

SEQ ID NO: 233 - DNA, 22 bp, synthetic construct
  source 1..22: mol_type = other DNA
tgtcaagcagagaatgggtact

SEQ ID NO: 234 - DNA, 28 bp, synthetic construct
  source 1..28: mol_type = other DNA
agttttacatttttaatgctcctttctt

SEQ ID NO: 235 - DNA, 21 bp, synthetic construct
  source 1..21: mol_type = other DNA
tgatgaggtgattttcgtgga

SEQ ID NO: 236 - DNA, 20 bp, synthetic construct
  source 1..20: mol_type = other DNA
ggcacggtcttcagagaagc

SEQ ID NO: 237 - DNA, 28 bp, synthetic construct
  source 1..28: mol_type = other DNA
acattattgccaacatgacttacttgat

SEQ ID NO: 238 - DNA, 23 bp, synthetic construct
  source 1..23: mol_type = other DNA
gctgcagtgggaccactattatc

SEQ ID NO: 239 - DNA, 20 bp, synthetic construct
  source 1..20: mol_type = other DNA
gctcccggaagacagtcccc

SEQ ID NO: 240 - DNA, 21 bp, synthetic construct
  source 1..21: mol_type = other DNA
ctggctggaccaagcccatca

SEQ ID NO: 241 - DNA, 21 bp, synthetic construct
  source 1..21: mol_type = other DNA
ctaggcgaggagctccagtcg

SEQ ID NO: 242 - DNA, 22 bp, synthetic construct
  source 1..22: mol_type = other DNA
  modified_base 20..22: mod_base = OTHER; note = methylated nucleotide
gatctgggactgcatgctggtg

SEQ ID NO: 243 - DNA, 23 bp, synthetic construct
  source 1..23: mol_type = other DNA
atgctgaccaactcgtgtgtgaa

SEQ ID NO: 244 - DNA, 23 bp, synthetic construct
  source 1..23: mol_type = other DNA
cagaactcgcaacagtccttcga

SEQ ID NO: 245 - DNA, 21 bp, synthetic construct
  source 1..21: mol_type = other DNA
gtgcgtggaggagatcgagcg

SEQ ID NO: 246 - DNA, 24 bp, synthetic construct
  source 1..24: mol_type = other DNA
tgattatagcctaagacccggagc

SEQ ID NO: 247 - DNA, 22 bp, synthetic construct
  source 1..22: mol_type = other DNA
cctaagacccggagcttttcac

(19) United States
(12) Patent Application Publication
(10) Pub. No.: US 2025/0290146 A1
(43) Pub. Date: Sep. 18, 2025

(54) METHODS AND COMPOSITIONS FOR SCREENING FOR MYELOPROLIFERATIVE DISORDERS

(71) Applicant: Precipio, Inc., New Haven, CT (US)

(72) Inventor: Ayman Mohamed, New Haven, CT (US)

(21) Appl. No.: 18/859,034

(22) PCT Filed: Apr. 24, 2023
(86) PCT No.: PCT/US2023/019643

§ 371 Date: Oct. 22, 2024

Related U.S. Application Data
(60) Provisional application No. 63/363,469, filed on Apr. 22, 2022.

(51) Int. Cl. C12Q 1/6886; C12Q 1/6851

(52) U.S. Cl. CPC C12Q 1/6886; C12Q 1/6851; C12Q 2600/156

(57) ABSTRACT

The invention relates to compositions and methods for determining risk of developing myeloproliferative disorders. In certain embodiments, the invention includes screening for mutations in the genes JAK2, MPL (thrombopoietin receptor), and CALR (calreticulin). In certain embodiments, the invention includes screening for mutations in the genes FLT3, IDH2, IDH1, KIT NPM1, and/or CEBPA, In certain embodiments, the invention includes screening for mutations in the genes WT1, ASXL1, RUNX1, DNMT3, and/or SF3B1.

### CROSS REFERENCE TO RELATED APPLICATIONS

This application claims the benefit of and priority to U.S. Provisional Application No. 63/363,469, filed Apr. 22, 2022, the entire disclosure of which is hereby incorporated by reference in its entirety for all purposes.

### FIELD OF THE INVENTION

The invention relates to compositions and methods for determining risk of developing myeloproliferative disorders. In certain embodiments, the invention includes screening for mutations in the genes MPL (thrombopoietin receptor), CALR (calreticulin), and JAK2 (Janus Kinase 2). In certain embodiments, the invention includes screening for mutations in the genes FLT3 (fms related receptor tyrosine kinase 3), IDH2 (isocitrate dehydrogenase (NADP(+)) 2), IDH1 (isocitrate dehydrogenase (NADP(+)) 1), KIT receptor tyrosine kinase, NPM1 (nucleophosmin), and/or CEBPA (CCAAT/enhancer-binding protein). In certain embodiments, the invention includes screening for mutations in the genes WT1 (Wilms' tumor gene 1), ASXL1 (additional sex combs gene), RUNX1 (runt-related transcription factor 1), DNMT3 (DNA methyltransferase 3), SF3B1 (splicing factor 3B subunit 1), and/or TERT (telomerase reverse transcriptase). In certain embodiments, the invention includes screening for mutation in the genes SF3B1 (splicing Factor 3b Subunit 1), NOTCH1 (Notch receptor 1), CXCR4 (C-X-C Motif Chemokine Receptor 4), and/or MYD88 (MYD88 Innate Immune Signal Transduction Adaptor). In certain embodiments, the invention includes screening for the presence of exons in the genes BCR (breakpoint cluster region) and ABL1 (abelson tyrosine kinase).

### BACKGROUND

Myelodysplastic syndromes (MDS) are a group of disorders in which immature blood cells (blast cells) formed in the bone marrow fail to properly mature. Instead of developing normally, the cells die in the bone marrow or shortly after entering the bloodstream. Individuals with MDS may suffer from fatigue, susceptibility to infection, and bleeding and bruising. In some cases, MDS progresses to acute myeloid leukemia (AML).

Myeloproliferative neoplasms (MPN) are disorders, including chronic myeloid leukemia (CML), polycythemia vera, essential thrombocythemia, and myelofibrosis, in which abnormal or too many blood cells are generated. Individuals with MPN may experience headaches, blurred vision, fatigue, weakness, dizziness, itchiness, high blood pressure, enlarged spleen or liver, night sweats and bone pain. In some cases, MPN progresses to acute myeloid leukemia (AML).

The diagnostic process for myelodysplastic syndromes (MDS) and myeloproliferative neoplasms (MPN) includes testing for mutations in genes known to give rise to such disorders, such as MPL (thrombopoietin receptor), CALR (calreticulin), and JAK2 (Janus Kinase 2). The known positive frequency for these mutations is low. However, expensive reagents and positive controls create a high cost of running these tests. Full mutation analysis by more specific post-PCR detection technologies (i.e., RT-PCR, Sanger, etc.) is currently performed on all samples being evaluated for MDS or MPN. To lower costs, these tests are often administered in batches, which can increase delay. Further, given the expense of the tests, they are often performed serially, starting with one mutation and, if that test is negative, moving on to the next mutation. The serial assessment of these mutations also contributes to delay in diagnosis. Thus, current approaches are costly, time consuming, and unnecessary for negative cases.

Accordingly, there is a clear, on-going, and urgent need to reduce time and cost of diagnosis of myeloproliferative disorders through methods that permit rapid, low-cost identification of mutations in key genes.

### SUMMARY

The present disclosure is based in part, upon the discovery of a rapid, low-cost method of detecting the presence or absence of a mutation in one or more of the genes JAK2 (Janus Kinase 2), MPL (thrombopoietin receptor), and CALR (calreticulin). In certain embodiments, the mutation is in exon 10 of the MPL gene, in exon 9 of the CALR gene, or in exon 12 or 14 of the JAK2 gene. The method may include amplifying (e.g., by PCR) a portion of one or more of the exons using the primers set forth in SEQ ID NOs: 1 and 2, SEQ ID NOs: 3 and 4, SEQ ID NOs: 5 and 6, SEQ ID NOs: 7 and 8, and SEQ ID NOs: 21 and 22 or primers having the reverse complement sequence to any of the foregoing, or primers having 90% sequence identity to any of the foregoing. In certain embodiments, the method includes the use of high resolution melting (HRM) to detect the presence or absence of the mutation.

The present disclosure also is based in part, upon the discovery of a rapid, low-cost method of detecting the presence or absence of a mutation in one or more of the genes FLT3 (fms related receptor tyrosine kinase 3), IDH2 (isocitrate dehydrogenase (NADP(+)) 2), IDH1 (isocitrate dehydrogenase (NADP(+)) 1), KIT receptor tyrosine kinase. NPM1 (nucleophosmin), and/or CEBPA (CCAAT/enhancer-binding protein). In certain embodiments, the mutation is in exon 14, 15, 16, or 20 of the FLT3 gene, in exon 4 of the IDH2 gene, in exon 4 of the IDF1 gene, in 9, 11, 13, 17 of the KIT gene, in exon 12 of the NPM1 gene, or in exon 1 of the CEBPA gene. The method may include amplifying (e.g., by PCR) a portion of one or more of the exons using the primers set forth in SEQ ID NOs: 26 and 27, SEQ ID NOs: 28 and 29, SEQ ID NOs: 30 and 31, SEQ ID NOs: 32 and 33, SEQ ID NOs: 234 and 235, SEQ ID NOs: 34 and 35, SEQ ID NOs: 36 and 37, SEQ ID NOs: 238 and 239, SEQ ID NOs: 240 and 241, SEQ ID NOs: 38 and 39, SEQ ID NOs: 236 and 237, SEQ ID NOs: 40 and 41, SEQ ID NOs: 42 and 43, SEQ ID NOs: 44 and 45, SEQ ID NOs: 46 and 47, SEQ ID NOs: 232 and 233, SEQ ID NOs: 48 and 49, SEQ ID NOs: 50 and 51, SEQ ID NOs: 52 and 53, SEQ ID NOs: 54 and 55, SEQ ID NOs: 56 and 57, SEQ ID NOs: 58 and 59, SEQ ID NOs: 60 and 61, and SEQ ID NOs: 62 and 63, or primers having the reverse complement sequence to any of the foregoing, or primers having 90% sequence identity to any of the foregoing. In certain embodiments, the method includes the use of high resolution melting (HRM) to detect the presence or absence of the mutation.

The present disclosure also is based in part, upon the discovery of a rapid, low-cost method of detecting the presence or absence of a mutation in one or more of the genes WT1 (Wilms' tumor gene 1), ASXL1 (additional sex combs gene), RUNX1 (runt-related transcription factor 1), DNMT3 (DNA methyltransferase 3), SF3B1 (splicing factor 3B subunit 1), and/or TERT (telomerase reverse transcriptase). In certain embodiments, the mutation is in exon 8 or 10 of the WT1 gene, in exon 12 of the ASXL1 gene, in exon 4, 5, 6, or 8 of the RUNX1 gene, in exon 23 of the DNMT3A gene, in exon 15, 16 & 17 of the SF3B1 gene, and/or in the TERT promoter or exon 15. In certain embodiments, the mutation is in exon 8 or 10 of the WT1 gene, in exon 12 of the ASXL1 gene, in exon 4, 5, 6, or 8 of the RUNX1 gene, in exon 23 of the DNMT3A gene, and/or in exon 15, 16 & 17 of the SF3B1 gene. The method may include amplifying (e.g., by PCR) a portion of one or more of the exons using the primers set forth in SEQ ID NOs: 64 and 65, SEQ ID NOs: 66 and 67, SEQ ID NOs: 216 and 217, SEQ ID NOs: 218 and 219, SEQ ID NOs: 68 and 69, SEQ ID NOs: 70 and 71, SEQ ID NOs: 72 and 73, SEQ ID NOs: 74 and 75, SEQ ID NOs: 76 and 77, SEQ ID NOs: 192 and 193, SEQ ID NOs: 194 and 195, SEQ ID NOs: 196 and 197, SEQ ID NOs: 198 and 199, SEQ ID NOs: 200 and 201SEQ ID NOs: 78 and 79, SEQ ID NOs: 80 and 81, SEQ ID NOs: 82 and 83, SEQ ID NOs: 84 and 85. SEQ ID NOs: 204 and 205, SEQ ID NOs: 206 and 207, SEQ ID NOs: 86 and 87, SEQ ID NOs: 202 and 203, SEQ ID NOs: 88 and 89, SEQ ID NOs: 90 and 91, SEQ ID NOs: 92 and 93, SEQ ID NOs: 208 and 209, SEQ ID NOs: 210 and 211, SEQ ID NOs: 212 and 213, SEQ ID NOs: 214 and 215, or primers having the reverse complement sequence to any of the foregoing, or primers having 90% sequence identity to any of the foregoing. In certain embodiments, the method includes the use of high resolution melting (HRM) to detect the presence or absence of the mutation.

The present disclosure also is based in part, upon the discovery of a rapid, low-cost method of detecting the presence or absence of a mutation in one or more of the genes SF3B1 (splicing Factor 3b Subunit 1), NOTCH1 (Notch receptor 1), CXCR4 (C-X-C Motif Chemokine Receptor 4), and/or MYD88 (MYD88 Innate Immune Signal Transduction Adaptor). In certain embodiments, the mutation is in exon 15, 16, or 17 of the SF3B1 gene, in exon 34 of the NOTCH1 gene, in exon 2 of the CXCR4 gene, or in exon 3, 4, or 5 of the MYD88 gene. The method may include amplifying (e.g., by PCR) a portion of one or more of the exons using the primers set forth in SEQ ID NOs: 88 and 89, SEQ ID NOs: 90 and 91, SEQ ID NOs: 92 and 93, SEQ ID NOs: 208 and 209, SEQ ID NOs: 210 and 211, SEQ ID NOs: 212 and 213, SEQ ID NOs: 214 and 215, SEQ ID NOs: 220 and 221, SEQ ID NOs: 222 and 223, SEQ ID NOs: 224 and 225, SEQ ID NOs: 226 and 227, SEQ ID NOs: 228 and 229, and SEQ ID NOs: 230 and 231, or primers having the reverse complement sequence to any of the foregoing, or primers having 90% sequence identity to any of the foregoing. In certain embodiments, the method includes the use of high resolution melting (HRM) to detect the presence or absence of the mutation.

The present disclosure also is based in part, upon the discovery of a rapid, low-cost method of detecting the presence or absence of an exon in the genes BCR (breakpoint cluster region) and ABL1 (abelson tyrosine kinase). In certain embodiments, the exon is 1, 13, or 19 of the BCR gene and/or exon 3 of the ABL1 gene. The method may include amplifying (e.g., by PCR) a portion of one or more of the exons using the primers set forth in SEQ ID NOs: 243-247 or primers having the reverse complement sequence to any of the foregoing, or primers having 90% sequence identity to any of the foregoing. In certain embodiments, the method includes the use of high resolution melting (HRM) to detect the presence or absence of the exon.

The methods described herein are advantageous, in part, because they allow a clinician to quickly and inexpensively (through use of HRM in contrast to direct sequencing), rule out the presence of mutations in these genes and/or presence of exons in these genes. Only when a sample tests positive for a mutation is the slower, more expensive step of sequencing needed to confirm the presence of the mutation.

Thus, in one aspect, the disclosure relates to a method for determining the presence or absence of a mutation in a gene of interest (e.g., JAK2, MPL and/or CALR). The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 1)
TAGCCTGGATCTCCTTGGTG
and
(SEQ ID NO: 2)
GCGGTACCTGTAGTGTGCAG;
(ii)
(SEQ ID NO: 3)
GCAGAGAAACAAATGAAGGACAAAC
and
(SEQ ID NO: 4)
CTTCCTCCTTGTCCTCCTCA;
(iii)
(SEQ ID NO: 5)
TTTCTTTGAAGCAGCAAGTATG
and
(SEQ ID NO: 6)
AGATGCTCTGAGAAAGGCATTA;
(iv)
(SEQ ID NO: 7)
CCAACCTCACCAACATTACAGAG
and
(SEQ ID NO: 8)
TCCAATGTCACATGAATGTAAATCAA;
(v)
(SEQ ID NO: 21)
CTCTTGAAGAATGAAAGCCTTGG
and
(SEQ ID NO: 22)
GTTTCTGTGTGCTTTATCCAGAACT;

(vi) a pair of primers having the reverse complement sequences of any of (i) through (v); and
(vii) a pair of primers having at least 90% sequence identity to any of (i) through (vi).

In another aspect, the disclosure relates to a method for determining the presence or absence of a mutation in a MPL gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 1)
TAGCCTGGATCTCCTTGGTG
and
(SEQ ID NO: 2)
GCGGTACCTGTAGTGTGCAG;

(ii) a pair of primers having the reverse complement sequences of (i); and
(iii) a pair of primers having at least 90% sequence identity to any of (i) through (ii).

In another aspect, the disclosure relates to a method for determining the presence or absence of a mutation in a CALR gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 3)
GCAGAGAAACAAATGAAGGACAAAC
and
(SEQ ID NO: 4)
CTTCCTCCTTGTCCTCCTCA;

(ii) a pair of primers having the reverse complement sequences of (i); and
(iii) a pair of primers having at least 90% sequence identity to any of (i) through (ii).

In another aspect, the disclosure relates to a method for determining the presence or absence of a mutation in a JAK2 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 5)
TTTCTTTGAAGCAGCAAGTATG
and
(SEQ ID NO: 6)
AGATGCTCTGAGAAAGGCATTA;
(ii)
(SEQ ID NO: 7)
CCAACCTCACCAACATTACAGAG
and
(SEQ ID NO: 8)
TCCAATGTCACATGAATGTAAATCAA;
(iii)
(SEQ ID NO: 21)
CTCTTGAAGAATGAAAGCCTTGG
and
(SEQ ID NO: 22)
GTTTCTGTGTGCTTTATCCAGAACT;

(iv) a pair of primers having the reverse complement sequences of any of (i) through (iii); and
(v) a pair of primers having at least 90% sequence identity to any of (i) through (iv).

In another aspect, the disclosure relates to a method for determining the presence or absence of a mutation in a gene of interest (e.g., FLT3, IDH2, IDH1, KIT NPM1, and/or CEBPA). The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 30)
TTACATTTTTAATGCTCCTTTCTTTGA
and
(SEQ ID NO: 31)
GATGAGGTGATTTTCGTGGAAG;
(iv)
(SEQ ID NO: 32)
CTTGTCACCCACGGGAAAGT
and
(SEQ ID NO: 33)
AAAATAAGTAGGAAATAGCAGCCTCA;
(v)
(SEQ ID NO: 234)
AGTTTTACATTTTTAATGCTCCTTTCTT
and
(SEQ ID NO: 235)
TGATGAGGTGATTTTCGTGGA;
(vi)
(SEQ ID NO: 34)
TCTCTGTCCTCACAGAGTTCAAGC
and
(SEQ ID NO: 35)
CTTGGTCCAGCCAGGGACTA;
(vii)
(SEQ ID NO: 36)
CAAAAACATCCCACGCCTAGT
and
(SEQ ID NO: 37)
TGGGGTGAAGACCATTTTGAA;
(viii)
(SEQ ID NO: 238)
GCTGCAGTGGGACCACTATTATC;
and
(SEQ ID NO: 239)
GCTCCCGGAAGACAGTCCCC;
(ix)
(SEQ ID NO: 240)
CTGGCTGGACCAAGCCCATCA;
and
(SEQ ID NO: 241)
CTAGGCGAGGAGCTCCAGTCG;
(x)
(SEQ ID NO: 38)
GAGAAGAGGGTTGAGGAGTTCAA
and
(SEQ ID NO: 39)
GCAAAATCACATTATTGCCAACAT;
(xi)
(SEQ ID NO: 236)
GGCACGGTCTTCAGAGAAGC;
and
(SEQ ID NO: 237)
ACATTATTGCCAACATGACTTACTTGAT;
(xii)
(SEQ ID NO: 40)
CTAGTGCATTCAAGCACAATGG
and
(SEQ ID NO: 41)
CAGAGCCTAAACATCCCCTTAAA;
(xii)
(SEQ ID NO: 42)
AGGTGATCTATTTTTCCCTTTCTCC
and
(SEQ ID NO: 43)
TTATGTGTACCCAAAAAGGTGACA;
(xiv)
(SEQ ID NO: 44)
TGCCAGTTGTGCTTTTTGCT
and
(SEQ ID NO: 45)
ACAATAAAAGGCAGCTTGGACAC;
(xv)
(SEQ ID NO: 46)
ACTTGGCAGCCAGAAATATCCTC
and
(SEQ ID NO: 47)
GACTGTCAAGCAGAGAATGGGTACT;
(xvi)
(SEQ ID NO: 232)
GTATTCACAGAGACTTGGCAGC;
and
(SEQ ID NO: 233)
TGTCAAGCAGAGAATGGGTACT;
(xvii)
(SEQ ID NO: 48)
GATGTCTATGAAGTGTTGTGGTTCC
and
(SEQ ID NO: 49)
TGTTACAGAAATGAAATAAGACGGAAA;
(xviii)
(SEQ ID NO: 50)
GAGGCTCGCCATGCCGGGAG
and
(SEQ ID NO: 51)
GATGTCGATGGACGTCTCGTGC;
(xix)
(SEQ ID NO: 52)
CAGCGCCGCCTTCGGCTTTC
and
(SEQ ID NO: 53)
GCCCGGGTAGTCAAAGTCGC;
(xx)
(SEQ ID NO: 54)
GGCAGCAGGAGAAGGCCAAG
and
(SEQ ID NO: 55)
TTCATCCTCCTCGCGGGGCTC;
(xxi)
(SEQ ID NO: 56)
CGGCCGCTGGTGATCAAGC
and
(SEQ ID NO: 57)
GGTGACCGGGCTGCAGGTG;
(xxii)
(SEQ ID NO: 58)
CGCACTGCGGCCAGACCAC
and
(SEQ ID NO: 59)
GTTGCTGTTCTTGTCCACCGAC;
(xxiii)
(SEQ ID NO: 60)
GCGGGCAAGGCCAAGAAGTC
and
(SEQ ID NO: 61)
GTGTCCAGTTCGCGGCTCAG;
(xxiv)
(SEQ ID NO: 62)
GACCAGTGACAATGACCGCCTG
and
(SEQ ID NO: 63)
GGCGACCCCAAACCACTCC;

(xxv) a pair of primers having the reverse complement sequences of any of (i) through (xxiv); and
(xxvi) a pair of primers having at least 90% sequence identity to any of (i) through (xxv).

In another aspect, the disclosure relates to a method for determining the presence or absence of a mutation in a FLT3 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 32)
CTTGTCACCCACGGGAAAGT
and
(SEQ ID NO: 33)
AAAATAAGTAGGAAATAGCAGCCTCA;
(iv)
(SEQ ID NO: 234)
AGTTTTACATTTTTAATGCTCCTTTCTT
and
(SEQ ID NO: 235)
TGATGAGGTGATTTTCGTGGA;

(v) a pair of primers having the reverse complement sequences of any of (i) through (iv); and
(vi) a pair of primers having at least 90% sequence identity to any of (i) through (v).

In another aspect, the disclosure relates to a method for determining the presence or absence of a mutation in a IDH2 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 34)
TCTCTGTCCTCACAGAGTTCAAGC
and
(SEQ ID NO: 35)
CTTGGTCCAGCCAGGGACTA;
(ii)
(SEQ ID NO: 36)
CAAAAACATCCCACGCCTAGT
and
(SEQ ID NO: 37)
TGGGGTGAAGACCATTTTGAA;
(iii)
(SEQ ID NO: 238)
GCTGCAGTGGGACCACTATTATC;
and
(SEQ ID NO: 239)
GCTCCCGGAAGACAGTCCCC;
(iv)
(SEQ ID NO: 240)
CTGGCTGGACCAAGCCCATCA;
and
(SEQ ID NO: 241)
CTAGGCGAGGAGCTCCAGTCG;

(v) a pair of primers having the reverse complement sequences of any of (i) through (iv); and
(vi) a pair of primers having at least 90% sequence identity to any of (i) through (v).

In another aspect, the disclosure relates to a method for determining the presence or absence of a mutation in a IDH1 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 38)
GAGAAGAGGGTTGAGGAGTTCAA
and
(SEQ ID NO: 39)
GCAAAATCACATTATTGCCAACAT;
(ii)
(SEQ ID NO: 236)
GGCACGGTCTTCAGAGAAGC;
and
(SEQ ID NO: 237)
ACATTATTGCCAACATGACTTACTTGAT;

(iii) a pair of primers having the reverse complement sequences of any of (i) through (ii); and
(iv) a pair of primers having at least 90% sequence identity to any of (i) through (iii).

In another aspect, the disclosure relates to a method for determining the presence or absence of a mutation in a KIT gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 40)
CTAGTGCATTCAAGCACAATGG
and
(SEQ ID NO: 41)
CAGAGCCTAAACATCCCCTTAAA;
(ii)
(SEQ ID NO: 42)
AGGTGATCTATTTTTCCCTTTCTCC
and
(SEQ ID NO: 43)
TTATGTGTACCCAAAAAGGTGACA;
(iii)
(SEQ ID NO: 44)
TGCCAGTTGTGCTTTTTGCT
and
(SEQ ID NO: 45)
ACAATAAAAGGCAGCTTGGACAC;
(v)
(SEQ ID NO: 232)
GTATTCACAGAGACTTGGCAGC;
and
(SEQ ID NO: 233)
TGTCAAGCAGAGAATGGGTACT;

(vi) a pair of primers having the reverse complement sequences of any of (i) through (v); and
(vii) a pair of primers having at least 90% sequence identity to any of (i) through (vi).

In another aspect, the disclosure relates to a method for determining the presence or absence of a mutation in a NMP1 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 48)
GATGTCTATGAAGTGTTGTGGTTCC
and
(SEQ ID NO: 49)
TGTTACAGAAATGAAATAAGACGGAAA;

(ii) a pair of primers having the reverse complement sequences of (i); and
(iii) a pair of primers having at least 90% sequence identity to any of (i) through (ii).

In another aspect, the disclosure relates to a method for determining the presence or absence of a mutation in a CEBPA gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 50)
GAGGCTCGCCATGCCGGGAG
and
(SEQ ID NO: 51)
GATGTCGATGGACGTCTCGTGC;
(ii)
(SEQ ID NO: 52)
CAGCGCCGCCTTCGGCTTTC
and
(SEQ ID NO: 53)
GCCCGGGTAGTCAAAGTCGC;
(iii)
(SEQ ID NO: 54)
GGCAGCAGGAGAAGGCCAAG
and
(SEQ ID NO: 55)
TTCATCCTCCTCGCGGGGCTC;
(iv)
(SEQ ID NO: 56)
CGGCCGCTGGTGATCAAGC
and
(SEQ ID NO: 57)
GGTGACCGGGCTGCAGGTG;
(v)
(SEQ ID NO: 58)
CGCACTGCGGCCAGACCAC
and
(SEQ ID NO: 59)
GTTGCTGTTCTTGTCCACCGAC;
(vi)
(SEQ ID NO: 60)
GCGGGCAAGGCCAAGAAGTC
and
(SEQ ID NO: 61)
GTGTCCAGTTCGCGGCTCAG;
(vii)
(SEQ ID NO: 62)
GACCAGTGACAATGACCGCCTG
and
(SEQ ID NO: 63)
GGCGACCCCAAACCACTCC;

(vii) a pair of primers having the reverse complement sequences of any of (i) through (vii); and
(ix) a pair of primers having at least 90% sequence identity to any of (i) through (viii).

In another aspect, the disclosure relates to a method for determining the presence or absence of a mutation in a gene of interest (e.g., WT1, ASXL1, RUNX1, DNMT3, SF3B1, and/or TERT). The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 64)
CCTTCCTCTTACTCTCTGCCTGC
and
(SEQ ID NO: 65)
GACAGCGGGCACACTTACCAG;
(ii)
(SEQ ID NO: 66)
CATTGTTAGGGCCGAGGCTAGA
and
(SEQ ID NO: 67)
ACGCACTTGTTTTACCTGTATGAGTC;
(iii)
(SEQ ID NO: 218)
GCCTCCCTTCCTCTTACTCTCT;
and
GCAGCCTGGGTAAGCACAC;
(iv)
(SEQ ID NO: 68)
GCAGATTCAACTTTCACGTATCAAACC
and
(SEQ ID NO: 69)
TGCAGAGCACGGGCTTTAATGT;
(v)
(SEQ ID NO: 70)
CCTCGCAGACATTAAAGCCCGT
and
(SEQ ID NO: 71)
CCACCATCACCACTGCTGCTG;
(vi)
(SEQ ID NO: 72)
CAGTGGTGATGGTGGTGAGGC
and
(SEQ ID NO: 73)
CCTAGCCCATCTGTGAGTCCAAC;
(vii)
(SEQ ID NO: 74)
TGTCCTCCCAAACCTCAGTAGC
and
(SEQ ID NO: 75)
AGCTTGGCCAGTTCCTTTCTCT;
(viii)
(SEQ ID NO: 76)
GTGTCTCGAGTATGTGCGGTCC
and
(SEQ ID NO: 77)
CATCGTGGGCTGGTGGAAGAA;
(ix)
(SEQ ID NO: 192)
CTTTCACGTATCAAACCACCC
and
(SEQ ID NO: 193)
CACGGGCTTTAATGTCTGC;
(x)
(SEQ ID NO: 194)
AAGATCCCAGATTCCCTACTG
and
(SEQ ID NO: 195)
GTGGGCTGGTGGAAGAAC;
(xi)
(SEQ ID NO: 196)
GGCGAGAGGTCACCACTG
and
(SEQ ID NO: 197)
CCATCACCACTGCTGCTG;
(xii)
(SEQ ID NO: 198)
AACTGAATGTGAGTCTGGCA
and
(SEQ ID NO: 199)
CACTAGAGACGGAATGGGAC;
(xiii)
(SEQ ID NO: 200)
CAGTGGTGATGGTGGTGAG
and
(SEQ ID NO: 201)
CCAACTGTAGCCCTCTGTAG;
(xiv)
(SEQ ID NO: 78)
TCCATTGCCTCTCCTTCTGTGC
and
(SEQ ID NO: 79)
TGCCAACTCCTTCATGCACCT;
(xv)
(SEQ ID NO: 80)
CCTAGGGGATGTTCCAGATGGC
and
(SEQ ID NO: 81)
AATGTGGGTTTGTTGCCATGAAAC;
(xvi)
(SEQ ID NO: 82)
TCTATCGTGTCCCCACAGGGAA
and
(SEQ ID NO: 83)
GGGGAAAGGTTGAACCCAAGGA;
(xvii)
(SEQ ID NO: 84)
CTTCACGCCGCCTTCCACCG
and
(SEQ ID NO: 85)
CTCCGGGCCAGTACCTTGAAA;
(xviii)
(SEQ ID NO: 204)
AGCAACGCCCATTTCACC
and
(SEQ ID NO: 205)
GCTCAGCTGCAAAGAATGTG;
(xix)
(SEQ ID NO: 206)
CAGGCAAGATGAGCGAGGC
and
(SEQ ID NO: 207)
GGGCCAGTACCTTGAAAGCG;
(xx)
(SEQ ID NO: 86)
TCCTGCTGTGTGGTTAGACGG
and
(SEQ ID NO: 87)
GAAGAGGTGGCGGATGACTGG;
(xxi)
(SEQ ID NO: 202)
CTGCTGTGTGGTTAGACGG
and
(SEQ ID NO: 203)
GAGGTGGCGGATGACTGG;
(xxii)
(SEQ ID NO: 88)
GCCCTGGGCATTCCTTCTTTATT
and
(SEQ ID NO: 89)
ACTTCTAAGATGTGGCAAGATGGC;
(xxiii)
(SEQ ID NO: 90)
GTTGGGGCATAGTTAAAACCTGTGT
and
(SEQ ID NO: 91)
TTCCTCTGTGTTGGCGGATACC;
(xxiv)
(SEQ ID NO: 92)
GGTATCCGCCAACACAGAGGAA
and
(SEQ ID NO: 93)
AGGAGACTGGAATTCTCGAATAAGGA;
(xxv)
(SEQ ID NO: 208)
GGCTGCTGGTCTGGCTACTAT
and
(SEQ ID NO: 209)
CCAGGGCAGAGGCTACAAC;
(xxvi)
(SEQ ID NO: 210)
TGTAGGTCTTGTGGATGAGC
and
(SEQ ID NO: 211)
TTCCTCTGTGTTGGCGGATA;
(xxvii)
(SEQ ID NO: 212)
CAGAGGAAAGGTAAATCCACCA
and
(SEQ ID NO: 213)
GGAATAAGATACCCAATAGCCTTCA;
(xxviii)
(SEQ ID NO: 214)
TTCCTTCTTTATTGCCCTTCTTA
and
(SEQ ID NO: 215)
CAACTTACCATGTTCAATGATTTCAA;

(xxix) a pair of primers having the reverse complement sequences of any of (i) through (xxviii); and
(xxx) a pair of primers having at least 90% sequence identity to any of (i) through (xxix);
In another aspect, the disclosure relates to a method for determining the presence or absence of a mutation in a WT1 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 218)
GCCTCCCTTCCTCTTACTCTCT;
and
GCAGCCTGGGTAAGCACAC;

(ii) a pair of primers having the reverse complement sequences of (i); and
(iii) a pair of primers having at least 90% sequence identity to any of (i) through (ii).

In another aspect, the disclosure relates to a method for determining the presence or absence of a mutation in a ASXL1 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 192)
CTTTCACGTATCAAACCACCC
and
(SEQ ID NO: 193)
CACGGGCTTTAATGTCTGC;
(ii)
(SEQ ID NO: 194)
AAGATCCCAGATTCCCTACTG
and
(SEQ ID NO: 195)
GTGGGCTGGTGGAAGAAC;
(iii)
(SEQ ID NO: 196)
GGCGAGAGGTCACCACTG
and
(SEQ ID NO: 197)
CCATCACCACTGCTGCTG;
(iv)
(SEQ ID NO: 198)
AACTGAATGTGAGTCTGGCA
and
(SEQ ID NO: 199)
CACTAGAGACGGAATGGGAC;
(v)
(SEQ ID NO: 200)
CAGTGGTGATGGTGGTGAG
and
(SEQ ID NO: 201)
CCAACTGTAGCCCTCTGTAG;

(vi) a pair of primers having the reverse complement sequences of any of (i) through (v); and
(vii) a pair of primers having at least 90% sequence identity to any of (i) through (vi).

In another aspect, the disclosure relates to a method for determining the presence or absence of a mutation in a RUNX1 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 204)
AGCAACGCCCATTTCACC
and
(SEQ ID NO: 205)
GCTCAGCTGCAAAGAATGTG;
(ii)
(SEQ ID NO: 206)
CAGGCAAGATGAGCGAGGC
and
(SEQ ID NO: 207)
GGGCCAGTACCTTGAAAGCG;

(iii) a pair of primers having the reverse complement sequences of any of (i) through (ii); and
(iv) a pair of primers having at least 90% sequence identity to any of (i) through (iii).

In another aspect, the disclosure relates to a method for determining the presence or absence of a mutation in a DNMT3A gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 202)
CTGCTGTGTGGTTAGACGG
and
(SEQ ID NO: 203)
GAGGTGGCGGATGACTGG;

(ii) a pair of primers having the reverse complement sequences of (i); and
(iii) a pair of primers having at least 90% sequence identity to any of (i) through (ii).

In another aspect, the disclosure relates to a method for determining the presence or absence of a mutation in a SFB31 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;

(v) a pair of primers having the reverse complement sequences of any of (i) through (iv); and
(vi) a pair of primers having at least 90% sequence identity to any of (i) through (v).

In another aspect, the disclosure relates to a method for determining the presence or absence of a mutation in a gene of interest (e.g., SF3B1, NOTCH1, CXCR4, and/or MYD88). The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 88)
GCCCTGGGCATTCCTTCTTTATT
and
(SEQ ID NO: 89)
ACTTCTAAGATGTGGCAAGATGGC;
(ii)
(SEQ ID NO: 90)
GTTGGGGCATAGTTAAAACCTGTGT
and
(SEQ ID NO: 91)
TTCCTCTGTGTTGGCGGATACC;
(iii)
(SEQ ID NO: 92)
GGTATCCGCCAACACAGAGGAA
and
(SEQ ID NO: 93)
AGGAGACTGGAATTCTCGAATAAGGA;
(iv)
(SEQ ID NO: 208)
GGCTGCTGGTCTGGCTACTAT
and
(SEQ ID NO: 209)
CCAGGGCAGAGGCTACAAC;
(v)
(SEQ ID NO: 210)
TGTAGGTCTTGTGGATGAGC
and
(SEQ ID NO: 211)
TTCCTCTGTGTTGGCGGATA;
(vi)
(SEQ ID NO: 212)
CAGAGGAAAGGTAAATCCACCA
and
(SEQ ID NO: 213)
GGAATAAGATACCCAATAGCCTTCA;
(vii)
(SEQ ID NO: 214)
TTCCTTCTTTATTGCCCTTCTTA
and
(SEQ ID NO: 215)
CAACTTACCATGTTCAATGATTTCAA;
(viii)
(SEQ ID NO: 230)
CTACTCCTCGCCTGTGGACAAC
and
(SEQ ID NO: 231)
GATCTGGGACTGCATGCTGGTG;
(ix)
(SEQ ID NO: 220)
CCAACGTCAGTGAGGCAGAT
and
(SEQ ID NO: 221)
GGATGACAATACCAGGCAGGA;
(x)
(SEQ ID NO: 222)
TGCTTTCCTTGGAGCCAAAT
and
(SEQ ID NO: 223)
TGTGTTAGCTGGAGTGAAAACTTG;
(xi)
(SEQ ID NO: 224)
ATATGCCTGAGCGTTTCGATGC
and
(SEQ ID NO: 225)
GTGGCCTTCTAGCCAACCTCT;
(xii)
(SEQ ID NO: 226)
CCCAGGGGATATGCTGAACTA
and
(SEQ ID NO: 227)
ACCTGGAGAGAGGCTGAGTG;
(xiii)
(SEQ ID NO: 228)
TTGGCTTGCAGGTGCCCATC
and
(SEQ ID NO: 229)
GGCGAGTCCAGAACCAAGATTT;

(xiv) a pair of primers having the reverse complement sequences of any of (i) through (xiii); and
(xv) a pair of primers having at least 90% sequence identity to any of (i) through (xiv);

In another aspect, the disclosure relates to a method for determining the presence or absence of a mutation in a SFB31 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;

(v) a pair of primers having the reverse complement sequences of any of (i) through (iv); and
(vi) a pair of primers having at least 90% sequence identity to any of (i) through (v).

In another aspect, the disclosure relates to a method for determining the presence or absence of a mutation in a NOTCH1 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 230)
CTACTCCTCGCCTGTGGACAAC
and
(SEQ ID NO: 231)
GATCTGGGACTGCATGCTGGTG;

(ii) a pair of primers having the reverse complement sequences of (i); and
(iii) a pair of primers having at least 90% sequence identity to any of (i) through (ii).

In another aspect, the disclosure relates to a method for determining the presence or absence of a mutation in a CXCR4 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 220)
CCAACGTCAGTGAGGCAGAT
and
(SEQ ID NO: 221)
GGATGACAATACCAGGCAGGA;
(ii)
(SEQ ID NO: 222)
TGCTTTCCTTGGAGCCAAAT
and
(SEQ ID NO: 223)
TGTGTTAGCTGGAGTGAAAACTTG;

(iii) a pair of primers having the reverse complement sequences of any of (i) through (ii); and
(iv) a pair of primers having at least 90% sequence identity to any of (i) through (iii).

In another aspect, the disclosure relates to a method for determining the presence or absence of a mutation in a MYD88 gene. The method can include amplifying a region of a gene of interest from a sample to produce a double-stranded oligonucleotide, wherein the amplifying step comprises performing PCR using a pair of primers. The pair of primers may be selected from the group consisting of:

(i)
(SEQ ID NO: 224)
ATATGCCTGAGCGTTTCGATGC
and
(SEQ ID NO: 225)
GTGGCCTTCTAGCCAACCTCT;
(ii)
(SEQ ID NO: 226)
CCCAGGGGATATGCTGAACTA
and
(SEQ ID NO: 227)
ACCTGGAGAGAGGCTGAGTG;
(iii)
(SEQ ID NO: 228)
TTGGCTTGCAGGTGCCCATC
and
(SEQ ID NO: 229)
GGCGAGTCCAGAACCAAGATTT;

(iv) a pair of primers having the reverse complement sequences of any of (i) through (iii); and
(v) a pair of primers having at least 90% sequence identity to any of (i) through (iv).

Thus, in one aspect, the disclosure relates to a method for determining the presence or absence of an exon in a gene of interest (e.g., BCR and/or ABL1). The method can include amplifying a region of a gene of interest from a sample to produce a double-stranded oligonucleotide, wherein the amplifying step comprises performing PCR using two or more primers. The primers may be selected from the group consisting of:

(i)
(SEQ ID NO: 243)
ATGCTGACCAACTCGTGTGTGAA;
(ii)
(SEQ ID NO: 244)
CAGAACTCGCAACAGTCCTTCGA;
(iii)
(SEQ ID NO: 245)
GTGCGTGGAGGAGATCGAGCG;
(iv)
(SEQ ID NO: 246)
TGATTATAGCCTAAGACCCGGAGC;
(v)
(SEQ ID NO: 247)
CCTAAGACCCGGAGCTTTTCAC;

(vi) two or more primers having the reverse complement sequences of any of (i) through (v); and
(vii) two or more primers having at least 90% sequence identity to any of (i) through (vi).

SEQ ID NO: 243 binds a portion of exon 13 of BCR, SEQ ID NO: 244 binds a portion of exon 1 of BCR, and SEQ ID NO: 245 binds a portion of exon 19 of BCR. SEQ ID NOs: 246 and 247 each binds a portion exon 3 of ABL1, SEQ ID NO: 243 can be used with either or both of SEQ ID NO: 246 or SEQ ID NO: 247 to amplify a gene encoding BCR/ABL1 isoform p203 or a gene encoding BCR/ABL1 isoform p 210, SEQ ID NO: 244 can be used with either or both of SEQ ID NO: 246 or SEQ ID NO: 247 to amplify a gene encoding BCR/ABL1 isoform p190. SEQ ID NO: 245 can be used with either or both of SEQ ID NO: 246 or SEQ ID NO: 247 to amplify a gene encoding BCR/ABL isoform p230.

In another aspect, the disclosure relates to a method for determining the presence or absence of an exon in a BCR/ABL1 fusion gene. The method can include amplifying a region of a gene of interest from a sample to produce a double-stranded oligonucleotide, wherein the amplifying step comprises performing PCR using two or more primers. The primers may be selected from the group consisting of:

(i)
(SEQ ID NO: 243)
ATGCTGACCAACTCGTGTGTGAA;
(ii)
(SEQ ID NO: 246)
TGATTATAGCCTAAGACCCGGAGC;
(iii)
(SEQ ID NO: 247)
CCTAAGACCCGGAGCTTTTCAC;

(iv) two or more primers having the reverse complement sequences of any of (i) through (iii); and
(v) two or more primers having at least 90% sequence identity to any of (i) through (iv).

In another aspect, the disclosure relates to a method for determining the presence or absence of an exon in a BCR/ABL1 fusion gene. The method can include amplifying a region of a gene of interest from a sample to produce a double-stranded oligonucleotide, wherein the amplifying step comprises performing PCR using two or more primers. The primers may be selected from the group consisting of:

(i)
(SEQ ID NO: 244)
CAGAACTCGCAACAGTCCTTCGA;
(ii)
(SEQ ID NO: 246)
TGATTATAGCCTAAGACCCGGAGC;
(iii)
(SEQ ID NO: 247)
CCTAAGACCCGGAGCTTTTCAC;

(iv) two or more primers having the reverse complement sequences of any of (i) through (iii); and
(v) two or more primers having at least 90% sequence identity to any of (i) through (iv).

In another aspect, the disclosure relates to a method for determining the presence or absence of an exon in a BCR/ABL1 fusion gene. The method can include amplifying a region of a gene of interest from a sample to produce a double-stranded oligonucleotide, wherein the amplifying step comprises performing PCR using two or more primers. The primers may be selected from the group consisting of:

(i)
(SEQ ID NO: 245)
GTGCGTGGAGGAGATCGAGCG;
(ii)
(SEQ ID NO: 246)
TGATTATAGCCTAAGACCCGGAGC;
(iii)
(SEQ ID NO: 247)
CCTAAGACCCGGAGCTTTTCAC;

(iv) two or more primers having the reverse complement sequences of any of (i) through (iii); and
(v) two or more primers having at least 90% sequence identity to any of (i) through (iv).

The method can further include determining that the double-stranded oligonucleotide comprises or does not comprise the mutation in the gene of interest by means of a High Resolution Melting (HRM) analysis.

In certain embodiments, the method further includes sequencing the mutation. In certain embodiments, the sample is from a subject in need of treatment of a myeloid malignancy, and the method further comprises treating the subject with an effective amount of a therapeutic agent to treat the myeloid malignancy. In certain embodiments, the sample is from a subject in need of treatment of acute myeloid leukemia, and the method further comprises treating the subject with an effective amount of a therapeutic agent to treat the acute myeloid leukemia. In certain embodiments, the sample is from a subject in need of treatment of anemia, and the method further comprises treating the subject with an effective amount of a therapeutic agent to treat anemia. In certain embodiments, the sample is from a subject in need of treatment of cytopenia, and the method further comprises treating the subject with an effective amount of a therapeutic agent to treat cytopenia. In certain embodiments, the sample is from a subject in need of treatment of chronic lymphocytic leukemia, and the method further comprises treating the subject with an effective amount of a therapeutic agent to treat chronic lymphocytic leukemia. In certain embodiments, the sample is from a subject in need of treatment of a BCR/ABL1-induced leukemogenesis (e.g., chronic myeloid leukemia (CML) or acute lymphocytic leukemia (ALL), and the method further comprises treating the subject with an effective amount of a therapeutic agent to treat BCR/ABL1-induced leukemogenesis (e.g., chronic myeloid leukemia (CML) or acute lymphocytic leukemia (ALL). In certain embodiments, the determining step includes comparing HRM analysis results of the double-stranded oligonucleotide to a control, for example, a control comprising HRM analysis results of a wild-type sample or a sample comprising the mutation of interest.

In certain embodiments, the HRM analysis is performed using a dye, for example, a dye comprising SYTO® 9, SYBR® Green, or Chai Green™.

In certain embodiments, the amplifying step comprises performing PCR using, in parallel, at least 2, 3, or 4 pairs of primers from (i) through (vii). In certain embodiments, the amplifying step comprises performing PCR using, in parallel, at least 2, 3, or 4 pairs of primers from (i) through (xxvi). In certain embodiments, the amplifying step comprises performing PCR using, in parallel, at least 2, 3, or 4 pairs of primers from (i) through (xxx). In certain embodiments, the amplifying step comprises performing PCR using, in parallel, at least 2, 3, or 4 pairs of primers from (i) through (xv). In certain embodiments, the amplifying step comprises performing PCR using, in parallel, at least 2, 3, or 4 pairs of primers from (i) through (v).

In certain embodiments, at least one of the pairs of primers comprises a tag. In certain embodiments, the tag comprises a sequencing primer such as an M13 primer. An M13 primer can include, for example, a M13 forward sequence or an M13 reverse sequence (e.g., AGGAAACAGCTATGACCAT (SEQ ID NO: 187) or TGTAAAACGACGGCCAGT (SEQ ID NO: 188)), or a complement or reverse complement thereof. In certain embodiments, the tag comprises a basic CG clamp sequence (e.g., GCGTCCCG (SEQ ID NO: 189): GCCCCCGCCG (SEQ ID NO: 190); or GCGGCCCGCCGCCCCCGCCG (SEQ ID NO: 191)).

In another aspect, the disclosure relates to a method of treating a subject in need of treatment of a myeloid malignancy. The method can include amplifying a region of a gene of interest from a sample from the subject to produce a double-stranded oligonucleotide, wherein the amplifying step comprises performing PCR using a pair of primers, selected from the group consisting of:

(i)
(SEQ ID NO: 1)
TAGCCTGGATCTCCTTGGTG
and
(SEQ ID NO: 2)
GCGGTACCTGTAGTGTGCAG;
(ii)
(SEQ ID NO: 3)
GCAGAGAAACAAATGAAGGACAAAC
and
(SEQ ID NO: 4)
CTTCCTCCTTGTCCTCCTCA;
(iii)
(SEQ ID NO: 5)
TTTCTTTGAAGCAGCAAGTATG
and
(SEQ ID NO: 6)
AGATGCTCTGAGAAAGGCATTA;
(iv)
(SEQ ID NO: 7)
CCAACCTCACCAACATTACAGAG
and
(SEQ ID NO: 8)
TCCAATGTCACATGAATGTAAATCAA;
(v)
(SEQ ID NO: 21)
CTCTTGAAGAATGAAAGCCTTGG
and
(SEQ ID NO: 22)
GTTTCTGTGTGCTTTATCCAGAACT;

(vi) a pair of primers having the reverse complement sequences of any of (i) through (v); and
(vii) a pair of primers having at least 90% sequence identity to any of (i) through (vi).

In another aspect, the disclosure relates to a method of treating a subject in need of treatment of a myeloid malignancy. The method can include amplifying a region of a MPL gene from a sample from the subject to produce a double-stranded oligonucleotide, wherein the amplifying step comprises performing PCR using a pair of primers, selected from the group consisting of:

(i)
(SEQ ID NO: 1)
TAGCCTGGATCTCCTTGGTG
and
(SEQ ID NO: 2)
GCGGTACCTGTAGTGTGCAG;

(ii) a pair of primers having the reverse complement sequences of (i); and
(iii) a pair of primers having at least 90% sequence identity to any of (i) through (ii).

In another aspect, the disclosure relates to a method of treating a subject in need of treatment of a myeloid malignancy. The method can include amplifying a region of a CALR gene from a sample from the subject to produce a double-stranded oligonucleotide, wherein the amplifying step comprises performing PCR using a pair of primers, selected from the group consisting of:

(i)
(SEQ ID NO: 3)
GCAGAGAAACAAATGAAGGACAAAC
and
(SEQ ID NO: 4)
CTTCCTCCTTGTCCTCCTCA;

(ii) a pair of primers having the reverse complement sequences of (i); and
(iii) a pair of primers having at least 90% sequence identity to any of (i) through (ii).

In another aspect, the disclosure relates to a method of treating a subject in need of treatment of a myeloid malignancy. The method can include amplifying a region of a JAK2 gene from a sample from the subject to produce a double-stranded oligonucleotide, wherein the amplifying step comprises performing PCR using a pair of primers, selected from the group consisting of:

(i)
(SEQ ID NO: 5)
TTTCTTTGAAGCAGCAAGTATG
and
(SEQ ID NO: 6)
AGATGCTCTGAGAAAGGCATTA;
(ii)
(SEQ ID NO: 7)
CCAACCTCACCAACATTACAGAG
and
(SEQ ID NO: 8)
TCCAATGTCACATGAATGTAAATCAA;
(iii)
(SEQ ID NO: 21)
CTCTTGAAGAATGAAAGCCTTGG
and
(SEQ ID NO: 22)
GTTTCTGTGTGCTTTATCCAGAACT;

(iv) a pair of primers having the reverse complement sequences of any of (i) through (iii); and
(v) a pair of primers having at least 90% sequence identity to any of (i) through (iv).

In another aspect, the disclosure relates to a method of treating a subject in need of treatment of acute myeloid leukemia. The method can include amplifying a region of a gene of interest from a sample from the subject to produce a double-stranded oligonucleotide, wherein the amplifying step comprises performing PCR using a pair of primers, selected from the group

(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 30)
TTACATTTTTAATGCTCCTTTCTTTGA
and
(SEQ ID NO: 31)
GATGAGGTGATTTTCGTGGAAG;
(iv)
(SEQ ID NO: 32)
CTTGTCACCCACGGGAAAGT
and
(SEQ ID NO: 33)
AAAATAAGTAGGAAATAGCAGCCTCA;
(v)
(SEQ ID NO: 234)
AGTTTTACATTTTTAATGCTCCTTTCTT
and
(SEQ ID NO: 235)
TGATGAGGTGATTTTCGTGGA;
(vi)
(SEQ ID NO: 34)
TCTCTGTCCTCACAGAGTTCAAGC
and
(SEQ ID NO: 35)
CTTGGTCCAGCCAGGGACTA;
(vii)
(SEQ ID NO: 36)
CAAAAACATCCCACGCCTAGT
and
(SEQ ID NO: 37)
TGGGGTGAAGACCATTTTGAA;
(viii)
(SEQ ID NO: 238)
GCTGCAGTGGGACCACTATTATC;
and
(SEQ ID NO: 239)
GCTCCCGGAAGACAGTCCCC;
(ix)
(SEQ ID NO: 240)
CTGGCTGGACCAAGCCCATCA;
and
(SEQ ID NO: 241)
CTAGGCGAGGAGCTCCAGTCG;
(x)
(SEQ ID NO: 38)
GAGAAGAGGGTTGAGGAGTTCAA
and
(SEQ ID NO: 39)
GCAAAATCACATTATTGCCAACAT;
(xi)
(SEQ ID NO: 236)
GGCACGGTCTTCAGAGAAGC;
and
(SEQ ID NO: 237)
ACATTATTGCCAACATGACTTACTTGAT;
(xii)
(SEQ ID NO: 40)
CTAGTGCATTCAAGCACAATGG
and
(SEQ ID NO: 41)
CAGAGCCTAAACATCCCCTTAAA;
(xii)
(SEQ ID NO: 42)
AGGTGATCTATTTTTCCCTTTCTCC
and
(SEQ ID NO: 43)
TTATGTGTACCCAAAAAGGTGACA;
(xiv)
(SEQ ID NO: 44)
TGCCAGTTGTGCTTTTTGCT
and
(SEQ ID NO: 45)
ACAATAAAAGGCAGCTTGGACAC;
(xv)
(SEQ ID NO: 46)
ACTTGGCAGCCAGAAATATCCTC
and
(SEQ ID NO: 47)
GACTGTCAAGCAGAGAATGGGTACT;
(xvi)
(SEQ ID NO: 232)
GTATTCACAGAGACTTGGCAGC;
and
(SEQ ID NO: 233)
TGTCAAGCAGAGAATGGGTACT;
(xvii)
(SEQ ID NO: 48)
GATGTCTATGAAGTGTTGTGGTTCC
and
(SEQ ID NO: 49)
TGTTACAGAAATGAAATAAGACGGAAA;
(xviii)
(SEQ ID NO: 50)
GAGGCTCGCCATGCCGGGAG
and
(SEQ ID NO: 51)
GATGTCGATGGACGTCTCGTGC;
(xix)
(SEQ ID NO: 52)
CAGCGCCGCCTTCGGCTTTC
and
(SEQ ID NO: 53)
GCCCGGGTAGTCAAAGTCGC;
(xx)
(SEQ ID NO: 54)
GGCAGCAGGAGAAGGCCAAG
and
(SEQ ID NO: 55)
TTCATCCTCCTCGCGGGGCTC;
(xxi)
(SEQ ID NO: 56)
CGGCCGCTGGTGATCAAGC
and
(SEQ ID NO: 57)
GGTGACCGGGCTGCAGGTG;
(xxii)
(SEQ ID NO: 58)
CGCACTGCGGCCAGACCAC
and
(SEQ ID NO: 59)
GTTGCTGTTCTTGTCCACCGAC;
(xxiii)
(SEQ ID NO: 60)
GCGGGCAAGGCCAAGAAGTC
and
(SEQ ID NO: 61)
GTGTCCAGTTCGCGGCTCAG;
(xxiv)
(SEQ ID NO: 62)
GACCAGTGACAATGACCGCCTG
and
(SEQ ID NO: 63)
GGCGACCCCAAACCACTCC;

(xxv) a pair of primers having the reverse complement sequences of any of (i) through (xxiv); and
(xxvi) a pair of primers having at least 90% sequence identity to any of (i) through (xxv);

In another aspect, the disclosure relates to a method of treating a subject in need of treatment of acute myeloid leukemia. The method can include amplifying a region of a FLT3 gene from a sample from the subject to produce a double-stranded oligonucleotide, wherein the amplifying step comprises performing PCR using a pair of primers, selected from the group

(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 32)
CTTGTCACCCACGGGAAAGT
and
(SEQ ID NO: 33)
AAAATAAGTAGGAAATAGCAGCCTCA;
(iv)
(SEQ ID NO: 234)
AGTTTTACATTTTTAATGCTCCTTTCTT
and
(SEQ ID NO: 235)
TGATGAGGTGATTTTCGTGGA;

(v) a pair of primers having the reverse complement sequences of any of (i) through (iv); and
(vi) a pair of primers having at least 90% sequence identity to any of (i) through (v).

In another aspect, the disclosure relates to a method of treating a subject in need of treatment of acute myeloid leukemia. The method can include amplifying a region of a IDH2 gene from a sample from the subject to produce a double-stranded oligonucleotide, wherein the amplifying step comprises performing PCR using a pair of primers, selected from the group consisting of:

(i)
(SEQ ID NO: 34)
TCTCTGTCCTCACAGAGTTCAAGC
and
(SEQ ID NO: 35)
CTTGGTCCAGCCAGGGACTA;
(ii)
(SEQ ID NO: 36)
CAAAAACATCCCACGCCTAGT
and
(SEQ ID NO: 37)
TGGGGTGAAGACCATTTTGAA;
(iii)
(SEQ ID NO: 238)
GCTGCAGTGGGACCACTATTATC;
and
(SEQ ID NO: 239)
GCTCCCGGAAGACAGTCCCC;
(iv)
(SEQ ID NO: 240)
CTGGCTGGACCAAGCCCATCA;
and
(SEQ ID NO: 241)
CTAGGCGAGGAGCTCCAGTCG;

(v) a pair of primers having the reverse complement sequences of any of (i) through (iv); and
(vi) a pair of primers having at least 90% sequence identity to any of (i) through (v).

In another aspect, the disclosure relates to a method of treating a subject in need of treatment of acute myeloid leukemia. The method can include amplifying a region of a IDH1 gene from a sample from the subject to produce a double-stranded oligonucleotide, wherein the amplifying step comprises performing PCR using a pair of primers, selected from the group consisting of:

(i)
(SEQ ID NO: 38)
GAGAAGAGGGTTGAGGAGTTCAA
and
(SEQ ID NO: 39)
GCAAAATCACATTATTGCCAACAT;
(ii)
(SEQ ID NO: 236)
GGCACGGTCTTCAGAGAAGC;
and
(SEQ ID NO: 237)
ACATTATTGCCAACATGACTTACTTGAT;

(iii) a pair of primers having the reverse complement sequences of any of (i) through (ii); and
(iv) a pair of primers having at least 90% sequence identity to any of (i) through (iii).

In another aspect, the disclosure relates to a method of treating a subject in need of treatment of acute myeloid leukemia. The method can include amplifying a region of a KIT gene from a sample from the subject to produce a double-stranded oligonucleotide, wherein the amplifying step comprises performing PCR using a pair of primers, selected from the group consisting of:

(i)
(SEQ ID NO: 40)
CTAGTGCATTCAAGCACAATGG
and
(SEQ ID NO: 41)
CAGAGCCTAAACATCCCCTTAAA;
(ii)
(SEQ ID NO: 42)
AGGTGATCTATTTTTCCCTTTCTCC
and
(SEQ ID NO: 43)
TTATGTGTACCCAAAAAGGTGACA;
(iii)
(SEQ ID NO: 44)
TGCCAGTTGTGCTTTTTGCT
and
(SEQ ID NO: 45)
ACAATAAAAGGCAGCTTGGACAC;
(v)
(SEQ ID NO: 232)
GTATTCACAGAGACTTGGCAGC;
and
(SEQ ID NO: 233)
TGTCAAGCAGAGAATGGGTACT;

(vi) a pair of primers having the reverse complement sequences of any of (i) through (v); and
(vii) a pair of primers having at least 90% sequence identity to any of (i) through (vi).

In another aspect, the disclosure relates to a method of treating a subject in need of treatment of acute myeloid leukemia. The method can include amplifying a region of a NMP1 gene from a sample from the subject to produce a double-stranded oligonucleotide, wherein the amplifying step comprises performing PCR using a pair of primers, selected from the group consisting of:

(i)
(SEQ ID NO: 48)
GATGTCTATGAAGTGTTGTGGTTCC
and
(SEQ ID NO: 49)
TGTTACAGAAATGAAATAAGACGGAAA;

(ii) a pair of primers having the reverse complement sequences of (i); and
(iii) a pair of primers having at least 90% sequence identity to any of (i) through (ii).

In another aspect, the disclosure relates to a method of treating a subject in need of treatment of acute myeloid leukemia. The method can include amplifying a region of a CEBPA gene from a sample from the subject to produce a double-stranded oligonucleotide, wherein the amplifying step comprises performing PCR using a pair of primers, selected from the group consisting of:

(i)
(SEQ ID NO: 50)
GAGGCTCGCCATGCCGGGAG
and
(SEQ ID NO: 51)
GATGTCGATGGACGTCTCGTGC;
(ii)
(SEQ ID NO: 52)
CAGCGCCGCCTTCGGCTTTC
and
(SEQ ID NO: 53)
GCCCGGGTAGTCAAAGTCGC;
(iii)
(SEQ ID NO: 54)
GGCAGCAGGAGAAGGCCAAG
and
(SEQ ID NO: 55)
TTCATCCTCCTCGCGGGGCTC;
(iv)
(SEQ ID NO: 56)
CGGCCGCTGGTGATCAAGC
and
(SEQ ID NO: 57)
GGTGACCGGGCTGCAGGTG;
(v)
(SEQ ID NO: 58)
CGCACTGCGGCCAGACCAC
and
(SEQ ID NO: 59)
GTTGCTGTTCTTGTCCACCGAC;
(vi)
(SEQ ID NO: 60)
GCGGGCAAGGCCAAGAAGTC
and
(SEQ ID NO: 61)
GTGTCCAGTTCGCGGCTCAG;
(vii)
(SEQ ID NO: 62)
GACCAGTGACAATGACCGCCTG
and
(SEQ ID NO: 63)
GGCGACCCCAAACCACTCC;

(vii) a pair of primers having the reverse complement sequences of any of (i) through (vii); and
(ix) a pair of primers having at least 90% sequence identity to any of (i) through (viii).

In another aspect, the disclosure relates to a method of treating a subject in need of treatment of anemia or cytopenia. The method can include amplifying a region of a gene of interest from a sample from the subject to produce a double-stranded oligonucleotide, wherein the amplifying step comprises performing PCR using a pair of primers, selected from the group

(i)
(SEQ ID NO: 64)
CCTTCCTCTTACTCTCTGCCTGC
and
(SEQ ID NO: 65)
GACAGCGGGCACACTTACCAG;
(ii)
(SEQ ID NO: 66)
CATTGTTAGGGCCGAGGCTAGA
and
(SEQ ID NO: 67)
ACGCACTTGTTTTACCTGTATGAGTC;
(iii)
(SEQ ID NO: 218)
GCCTCCCTTCCTCTTACTCTCT;
and
GCAGCCTGGGTAAGCACAC;
(iv)
(SEQ ID NO: 68)
GCAGATTCAACTTTCACGTATCAAACC
and
(SEQ ID NO: 69)
TGCAGAGCACGGGCTTTAATGT;
(v)
(SEQ ID NO: 70)
CCTCGCAGACATTAAAGCCCGT
and
(SEQ ID NO: 71)
CCACCATCACCACTGCTGCTG;
(vi)
(SEQ ID NO: 72)
CAGTGGTGATGGTGGTGAGGC
and
(SEQ ID NO: 73)
CCTAGCCCATCTGTGAGTCCAAC;
(vii)
(SEQ ID NO: 74)
TGTCCTCCCAAACCTCAGTAGC
and
(SEQ ID NO: 75)
AGCTTGGCCAGTTCCTTTCTCT;
(viii)
(SEQ ID NO: 76)
GTGTCTCGAGTATGTGCGGTCC
and
(SEQ ID NO: 77)
CATCGTGGGCTGGTGGAAGAA;
(ix)
(SEQ ID NO: 192)
CTTTCACGTATCAAACCACCC
and
(SEQ ID NO: 193)
CACGGGCTTTAATGTCTGC;
(x)
(SEQ ID NO: 194)
AAGATCCCAGATTCCCTACTG
and
(SEQ ID NO: 195)
GTGGGCTGGTGGAAGAAC;
(xi)
(SEQ ID NO: 196)
GGCGAGAGGTCACCACTG
and
(SEQ ID NO: 197)
CCATCACCACTGCTGCTG;
(xii)
(SEQ ID NO: 198)
AACTGAATGTGAGTCTGGCA
and
(SEQ ID NO: 199)
CACTAGAGACGGAATGGGAC;
(xiii)
(SEQ ID NO: 200)
CAGTGGTGATGGTGGTGAG
and
(SEQ ID NO: 201)
CCAACTGTAGCCCTCTGTAG;
(xiv)
(SEQ ID NO: 78)
TCCATTGCCTCTCCTTCTGTGC
and
(SEQ ID NO: 79)
TGCCAACTCCTTCATGCACCT;
(xv)
(SEQ ID NO: 80)
CCTAGGGGATGTTCCAGATGGC
and
(SEQ ID NO: 81)
AATGTGGGTTTGTTGCCATGAAAC;
(xvi)
(SEQ ID NO: 82)
TCTATCGTGTCCCCACAGGGAA
and
(SEQ ID NO: 83)
GGGGAAAGGTTGAACCCAAGGA;
(xvii)
(SEQ ID NO: 84)
CTTCACGCCGCCTTCCACCG
and
(SEQ ID NO: 85)
CTCCGGGCCAGTACCTTGAAA;
(xviii)
(SEQ ID NO: 204)
AGCAACGCCCATTTCACC
and
(SEQ ID NO: 205)
GCTCAGCTGCAAAGAATGTG;
(xix)
(SEQ ID NO: 206)
CAGGCAAGATGAGCGAGGC
and
(SEQ ID NO: 207)
GGGCCAGTACCTTGAAAGCG;
(xx)
(SEQ ID NO: 86)
TCCTGCTGTGTGGTTAGACGG
and
(SEQ ID NO: 87)
GAAGAGGTGGCGGATGACTGG;
(xxi)
(SEQ ID NO: 202)
CTGCTGTGTGGTTAGACGG
and
(SEQ ID NO: 203)
GAGGTGGCGGATGACTGG;
(xxii)
(SEQ ID NO: 88)
GCCCTGGGCATTCCTTCTTTATT
and
(SEQ ID NO: 89)
ACTTCTAAGATGTGGCAAGATGGC;
(xxiii)
(SEQ ID NO: 90)
GTTGGGGCATAGTTAAAACCTGTGT
and
(SEQ ID NO: 91)
TTCCTCTGTGTTGGCGGATACC;
(xxiv)
(SEQ ID NO: 92)
GGTATCCGCCAACACAGAGGAA
and
(SEQ ID NO: 93)
AGGAGACTGGAATTCTCGAATAAGGA;
(xxv)
(SEQ ID NO: 208)
GGCTGCTGGTCTGGCTACTAT
and
(SEQ ID NO: 209)
CCAGGGCAGAGGCTACAAC;
(xxvi)
(SEQ ID NO: 210)
TGTAGGTCTTGTGGATGAGC
and
(SEQ ID NO: 211)
TTCCTCTGTGTTGGCGGATA;
(xxvii)
(SEQ ID NO: 212)
CAGAGGAAAGGTAAATCCACCA
and
(SEQ ID NO: 213)
GGAATAAGATACCCAATAGCCTTCA;
(xxviii)
(SEQ ID NO: 214)
TTCCTTCTTTATTGCCCTTCTTA
and
(SEQ ID NO: 215)
CAACTTACCATGTTCAATGATTTCAA;

(xxix) a pair of primers having the reverse complement sequences of any of (i) through (xxviii); and
(xxx) a pair of primers having at least 90% sequence identity to any of (i) through (xxix).

In another aspect, the disclosure relates to a method of treating a subject in need of treatment of anemia or cytopenia. The method can include amplifying a region of a WT1 gene from a sample from the subject to produce a double-stranded oligonucleotide, wherein the amplifying step comprises performing PCR using a pair of primers, selected from the group consisting of:

(i)
(SEQ ID NO: 218)
GCCTCCCTTCCTCTTACTCTCT;
and
GCAGCCTGGGTAAGCACAC;

(ii) a pair of primers having the reverse complement sequences of (i); and
(iii) a pair of primers having at least 90% sequence identity to any of (i) through (ii).

In another aspect, the disclosure relates to a method of treating a subject in need of treatment of anemia or cytopenia. The method can include amplifying a region of a ASXL1 gene from a sample from the subject to produce a double-stranded oligonucleotide, wherein the amplifying step comprises performing PCR using a pair of primers, selected from the group

(i)
(SEQ ID NO: 192)
CTTTCACGTATCAAACCACCC
and
(SEQ ID NO: 193)
CACGGGCTTTAATGTCTGC;
(ii)
(SEQ ID NO: 194)
AAGATCCCAGATTCCCTACTG
and
(SEQ ID NO: 195)
GTGGGCTGGTGGAAGAAC;
(iii)
(SEQ ID NO: 196)
GGCGAGAGGTCACCACTG
and
(SEQ ID NO: 197)
CCATCACCACTGCTGCTG;
(iv)
(SEQ ID NO: 198)
AACTGAATGTGAGTCTGGCA
and
(SEQ ID NO: 199)
CACTAGAGACGGAATGGGAC;
(v)
(SEQ ID NO: 200)
CAGTGGTGATGGTGGTGAG
and
(SEQ ID NO: 201)
CCAACTGTAGCCCTCTGTAG;

(vi) a pair of primers having the reverse complement sequences of any of (i) through (v); and
(vii) a pair of primers having at least 90% sequence identity to any of (i) through (vi).

In another aspect, the disclosure relates to a method of treating a subject in need of treatment of anemia or cytopenia. The method can include amplifying a region of a RUNX1 gene from a sample from the subject to produce a double-stranded oligonucleotide, wherein the amplifying step comprises performing PCR using a pair of primers, selected from the group consisting of:

(ii)
(SEQ ID NO: 204)
AGCAACGCCCATTTCACC
and
(SEQ ID NO: 205)
GCTCAGCTGCAAAGAATGTG;
(ii)
(SEQ ID NO: 206)
CAGGCAAGATGAGCGAGGC
and
(SEQ ID NO: 207)
GGGCCAGTACCTTGAAAGCG;

(iii) a pair of primers having the reverse complement sequences of any of (i) through (ii); and
(iv) a pair of primers having at least 90% sequence identity to any of (i) through (iii).

In another aspect, the disclosure relates to a method of treating a subject in need of treatment of anemia or cytopenia. The method can include amplifying a region of a DNMT3A gene from a sample from the subject to produce a double-stranded oligonucleotide, wherein the amplifying step comprises performing PCR using a pair of primers, selected from the group consisting of:

(i)
(SEQ ID NO: 202)
CTGCTGTGTGGTTAGACGG
and
(SEQ ID NO: 203)
GAGGTGGCGGATGACTGG;

(ii) a pair of primers having the reverse complement sequences of (i); and
(iii) a pair of primers having at least 90% sequence identity to any of (i) through (ii).

In another aspect, the disclosure relates to a method of treating a subject in need of treatment of anemia or cytopenia. The method can include amplifying a region of a SFB31 gene from a sample from the subject to produce a double-stranded oligonucleotide, wherein the amplifying step comprises performing PCR using a pair of primers, selected from the group consisting of:

(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;

(v) a pair of primers having the reverse complement sequences of any of (i) through (iv); and
(vi) a pair of primers having at least 90% sequence identity to any of (i) through (v).

In another aspect, the disclosure relates to a method of treating a subject in need of treatment of chronic lymphocytic leukemia. The method can include amplifying a region of a gene of interest from a sample from the subject to produce a double-stranded oligonucleotide, wherein the amplifying step comprises performing PCR using a pair of primers, selected from the group consisting of:

(i)
(SEQ ID NO: 88)
GCCCTGGGCATTCCTTCTTTATT
and
(SEQ ID NO: 89)
ACTTCTAAGATGTGGCAAGATGGC;
(ii)
(SEQ ID NO: 90)
GTTGGGGCATAGTTAAAACCTGTGT
and
(SEQ ID NO: 91)
TTCCTCTGTGTTGGCGGATACC;
(iii)
(SEQ ID NO: 92)
GGTATCCGCCAACACAGAGGAA
and
(SEQ ID NO: 93)
AGGAGACTGGAATTCTCGAATAAGGA;
(iv)
(SEQ ID NO: 208)
GGCTGCTGGTCTGGCTACTAT
and
(SEQ ID NO: 209)
CCAGGGCAGAGGCTACAAC;
(v)
(SEQ ID NO: 210)
TGTAGGTCTTGTGGATGAGC
and
(SEQ ID NO: 211)
TTCCTCTGTGTTGGCGGATA;
(vi)
(SEQ ID NO: 212)
CAGAGGAAAGGTAAATCCACCA
and
(SEQ ID NO: 213)
GGAATAAGATACCCAATAGCCTTCA;
(vii)
(SEQ ID NO: 214)
TTCCTTCTTTATTGCCCTTCTTA
and
(SEQ ID NO: 215)
CAACTTACCATGTTCAATGATTTCAA;
(viii)
(SEQ ID NO: 230)
CTACTCCTCGCCTGTGGACAAC
and
(SEQ ID NO: 231)
GATCTGGGACTGCATGCTGGTG;
(ix)
(SEQ ID NO: 220)
CCAACGTCAGTGAGGCAGAT
and
(SEQ ID NO: 221)
GGATGACAATACCAGGCAGGA;
(x)
(SEQ ID NO: 222)
TGCTTTCCTTGGAGCCAAAT
and
(SEQ ID NO: 223)
TGTGTTAGCTGGAGTGAAAACTTG;
(xi)
(SEQ ID NO: 224)
ATATGCCTGAGCGTTTCGATGC
and
(SEQ ID NO: 225)
GTGGCCTTCTAGCCAACCTCT;
(xii)
(SEQ ID NO: 226)
CCCAGGGGATATGCTGAACTA
and
(SEQ ID NO: 227)
ACCTGGAGAGAGGCTGAGTG;
(xiii)
(SEQ ID NO: 228)
TTGGCTTGCAGGTGCCCATC
and
(SEQ ID NO: 229)
GGCGAGTCCAGAACCAAGATTT;

(xiv) a pair of primers having the reverse complement sequences of any of (i) through (xiii); and
(xv) a pair of primers having at least 90% sequence identity to any of (i) through (xiv).

In another aspect, the disclosure relates to a method of treating a subject in need of treatment of chronic lymphocytic leukemia. The method can include amplifying a region of a SFB31 gene from a sample from the subject to produce a double-stranded oligonucleotide, wherein the amplifying step comprises performing PCR using a pair of primers, selected from the group consisting of:

(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;

(v) a pair of primers having the reverse complement sequences of any of (i) through (iv); and
(vi) a pair of primers having at least 90% sequence identity to any of (i) through (v).

In another aspect, the disclosure relates to a method of treating a subject in need of treatment of chronic lymphocytic leukemia. The method can include amplifying a region of a NOTCH1 gene from a sample from the subject to produce a double-stranded oligonucleotide, wherein the amplifying step comprises performing PCR using a pair of primers, selected from the group consisting of:

(i)
(SEQ ID NO: 230)
CTACTCCTCGCCTGTGGACAAC
and
(SEQ ID NO: 231)
GATCTGGGACTGCATGCTGGTG;

(ii) a pair of primers having the reverse complement sequences of (i); and
(iii) a pair of primers having at least 90% sequence identity to any of (i) through (ii).

In another aspect, the disclosure relates to a method of treating a subject in need of treatment of chronic lymphocytic leukemia. The method can include amplifying a region of a CXCR4 gene from a sample from the subject to produce a double-stranded oligonucleotide, wherein the amplifying step comprises performing PCR using a pair of primers, selected from the group consisting of:

(i)
(SEQ ID NO: 220)
CCAACGTCAGTGAGGCAGAT
and
(SEQ ID NO: 221)
GGATGACAATACCAGGCAGGA;
(ii)
(SEQ ID NO: 222)
TGCTTTCCTTGGAGCCAAAT
and
(SEQ ID NO: 223)
TGTGTTAGCTGGAGTGAAAACTTG;

(iii) a pair of primers having the reverse complement sequences of any of (i) through (ii); and
(iv) a pair of primers having at least 90% sequence identity to any of (i) through (iii).

In another aspect, the disclosure relates to a method of treating a subject in need of treatment of chronic lymphocytic leukemia. The method can include amplifying a region of a MYD88 gene from a sample from the subject to produce a double-stranded oligonucleotide, wherein the amplifying step comprises performing PCR using a pair of primers, selected from the group consisting of:

(i)
(SEQ ID NO: 224)
ATATGCCTGAGCGTTTCGATGC
and
(SEQ ID NO: 225)
GTGGCCTTCTAGCCAACCTCT;
(ii)
(SEQ ID NO: 226)
CCCAGGGGATATGCTGAACTA
and
(SEQ ID NO: 227)
ACCTGGAGAGAGGCTGAGTG;
(iii)
(SEQ ID NO: 228)
TTGGCTTGCAGGTGCCCATC
and
(SEQ ID NO: 229)
GGCGAGTCCAGAACCAAGATTT;

(iv) a pair of primers having the reverse complement sequences of any of (i) through (iii); and
(v) a pair of primers having at least 90% sequence identity to any of (i) through (iv).

In another aspect, the disclosure relates to a method of treating a subject in need of treatment of a BCR/ABL1-induced leukemogenesis malignancy. The method can include amplifying a region of a gene of interest from a sample from the subject to produce a double-stranded oligonucleotide, wherein the amplifying step comprises performing PCR using two or more primers, selected from the group consisting of:

(i)
(SEQ ID NO: 243)
ATGCTGACCAACTCGTGTGTGAA;
(ii)
(SEQ ID NO: 244)
CAGAACTCGCAACAGTCCTTCGA;
(iii)
(SEQ ID NO: 245)
GTGCGTGGAGGAGATCGAGCG;
(iv)
(SEQ ID NO: 246)
TGATTATAGCCTAAGACCCGGAGC;
(v)
(SEQ ID NO: 247)
CCTAAGACCCGGAGCTTTTCAC;

(vi) two or more primers having the reverse complement sequences of any of (i) through (v); and
(vii) two or more primers having at least 90% sequence identity to any of (i) through (vi).

In another aspect, the disclosure relates to a method of treating a subject in need of treatment of a BCR/ABL1-induced leukemogenesis malignancy. The method can include amplifying a region of a BCR/ABL1 fusion gene from a sample from the subject to produce a double-stranded oligonucleotide, wherein the amplifying step comprises performing PCR using a two or more primers, selected from the group consisting of:

(i)
(SEQ ID NO: 243)
ATGCTGACCAACTCGTGTGTGAA;
(ii)
(SEQ ID NO: 246)
TGATTATAGCCTAAGACCCGGAGC;
(iii)
(SEQ ID NO: 247)
CCTAAGACCCGGAGCTTTTCAC;

(iv) two or more primers having the reverse complement sequences of any of (i) through (iii); and
(v) two or more primers having at least 90% sequence identity to any of (i) through (iv).

In another aspect, the disclosure relates to a method of treating a subject in need of treatment of a BCR/ABL1-induced leukemogenesis malignancy. The method can include amplifying a region of a BCR/ABL1 fusion gene from a sample from the subject to produce a double-stranded oligonucleotide, wherein the amplifying step comprises performing PCR using a two or more primers, selected from the group consisting of:

(i)
(SEQ ID NO: 244)
CAGAACTCGCAACAGTCCTTCGA;
(ii)
(SEQ ID NO: 246)
TGATTATAGCCTAAGACCCGGAGC;
(iii)
(SEQ ID NO: 247)
CCTAAGACCCGGAGCTTTTCAC;

(iv) two or more primers having the reverse complement sequences of any of (i) through (iii); and
(v) two or more primers having at least 90% sequence identity to any of (i) through (iv).

In another aspect, the disclosure relates to a method of treating a subject in need of treatment of a BCR/ABL1-induced leukemogenesis malignancy. The method can include amplifying a region of a BCR/ABL1 fusion gene from a sample from the subject to produce a double-stranded oligonucleotide, wherein the amplifying step comprises performing PCR using a two or more primers, selected from the group consisting of:

(i)
(SEQ ID NO: 245)
GTGCGTGGAGGAGATCGAGCG;
(ii)
(SEQ ID NO: 246)
TGATTATAGCCTAAGACCCGGAGC;
(iii)
(SEQ ID NO: 247)
CCTAAGACCCGGAGCTTTTCAC;

(iv) two or more primers having the reverse complement sequences of any of (i) through (iii); and
(v) two or more primers having at least 90% sequence identity to any of (i) through (iv).

The method can also include determining that the double-stranded oligonucleotide comprises or does not comprise the mutation in the gene of interest by means of a High Resolution Melting (HRM) analysis. Where the mutation is present, the method can also include sequencing the mutation. The method can also include treating the subject in need of treatment of the myeloid malignancy with an effective amount of a therapeutic agent. The method can also include treating the subject in need of treatment of acute myeloid leukemia with an effective amount of a therapeutic agent. The method can also include treating the subject in need of treatment of the anemia with an effective amount of a therapeutic agent. The method can also include treating the subject in need of treatment of the cytopenia with an effective amount of a therapeutic agent. The method can also include treating the subject in need of treatment of the chronic lymphocytic leukemia with an effective amount of a therapeutic agent. The method can also include treating the subject in need of treatment of the BCR/ABL1-induced leukemogenesis (e.g., chronic myeloid leukemia (CML) or acute lymphocytic leukemia (ALL)) with an effective amount of a therapeutic agent.

In certain embodiments, the determining step includes comparing HRM analysis results of the double-stranded oligonucleotide to a control, for example, a control comprising HRM analysis results of a wild-type sample or a sample comprising the mutation of interest.

In certain embodiments, the HRM analysis is performed using a dye, for example, a dye comprising SYTO® 9, SYBR® Green, or Chai Green™. In certain embodiments, the dye is used at 2× concentration.

In certain embodiments, the amplifying step comprises performing PCR using, in parallel, at least 2, 3, or 4 pairs of primers from (i) through (vi). In certain embodiments, the amplifying step comprises performing PCR using, in parallel, at least 2, 3, or 4 pairs of primers from (i) through (xxvi). In certain embodiments, the amplifying step comprises performing PCR using, in parallel, at least 2, 3, or 4 pairs of primers from (i) through (xxx). In certain embodiments, the amplifying step comprises performing PCR using, in parallel, at least 2, 3, or 4 pairs of primers from (i) through (xv). In certain embodiments, the amplifying step comprises performing PCR using, in parallel, at least 2, 3, or 4 pairs of primers from (i) through (v).

In another aspect, the disclosure relates to a kit comprising a pair of primers capable of amplifying a region of a gene of interest from a sample to produce a double-stranded oligonucleotide, wherein the pair of primers is selected from the group consisting of:

(i)
(SEQ ID NO: 1)
TAGCCTGGATCTCCTTGGTG
and
(SEQ ID NO: 2)
GCGGTACCTGTAGTGTGCAG;
(ii)
(SEQ ID NO: 3)
GCAGAGAAACAAATGAAGGACAAAC
and
(SEQ ID NO: 4)
CTTCCTCCTTGTCCTCCTCA;
(iii)
(SEQ ID NO: 5)
TTTCTTTGAAGCAGCAAGTATG
and
(SEQ ID NO: 6)
AGATGCTCTGAGAAAGGCATTA;
(iv)
(SEQ ID NO: 7)
CCAACCTCACCAACATTACAGAG
and
(SEQ ID NO: 8)
TCCAATGTCACATGAATGTAAATCAA;
(v)
(SEQ ID NO: 21)
CTCTTGAAGAATGAAAGCCTTGG
and
(SEQ ID NO: 22)
GTTTCTGTGTGCTTTATCCAGAACT;

(vi) a pair of primers having the reverse complement sequences of any of (i) through (v); and
(vii) a pair of primers having at least 90% sequence identity to any of (i) through (vi).

In another aspect, the disclosure relates to a kit comprising a pair of primers capable of amplifying a region of a MPL gene from a sample to produce a double-stranded oligonucleotide, wherein the pair of primers is selected from the group consisting of:

(i)
(SEQ ID NO: 1)
TAGCCTGGATCTCCTTGGTG
and
(SEQ ID NO: 2)
GCGGTACCTGTAGTGTGCAG;

(ii) a pair of primers having the reverse complement sequences of (i); and
(iii) a pair of primers having at least 90% sequence identity to any of (i) through (ii).

In another aspect, the disclosure relates to a kit comprising a pair of primers capable of amplifying a region of a CALR gene from a sample to produce a double-stranded oligonucleotide, wherein the pair of primers is selected from the group consisting of:

(i)
(SEQ ID NO: 3)
GCAGAGAAACAAATGAAGGACAAAC
and
(SEQ ID NO: 4)
CTTCCTCCTTGTCCTCCTCA;

(ii) a pair of primers having the reverse complement sequences of (i); and
(iii) a pair of primers having at least 90% sequence identity to any of (i) through (ii).

In another aspect, the disclosure relates to a kit comprising a pair of primers capable of amplifying a region of a JAK2 gene from a sample to produce a double-stranded oligonucleotide, wherein the pair of primers is selected from the group consisting of:

(i)
(SEQ ID NO: 5)
TTTCTTTGAAGCAGCAAGTATG
and
(SEQ ID NO: 6)
AGATGCTCTGAGAAAGGCATTA;
(ii)
(SEQ ID NO: 7)
CCAACCTCACCAACATTACAGAG
and
(SEQ ID NO: 8)
TCCAATGTCACATGAATGTAAATCAA;
(iii)
(SEQ ID NO: 21)
CTCTTGAAGAATGAAAGCCTTGG
and
(SEQ ID NO: 22)
GTTTCTGTGTGCTTTATCCAGAACT;

(iv) a pair of primers having the reverse complement sequences of any of (i) through (iii); and
(v) a pair of primers having at least 90% sequence identity to any of (i) through (iv).

In another aspect, the disclosure relates to a kit comprising a pair of primers capable of amplifying a region of a gene of interest from a sample to produce a double-stranded oligonucleotide, wherein the pair of primers is selected from the group consisting of:

(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 30)
TTACATTTTTAATGCTCCTTTCTTTGA
and
(SEQ ID NO: 31)
GATGAGGTGATTTTCGTGGAAG;
(iv)
(SEQ ID NO: 32)
CTTGTCACCCACGGGAAAGT
and
(SEQ ID NO: 33)
AAAATAAGTAGGAAATAGCAGCCTCA;
(v)
(SEQ ID NO: 234)
AGTTTTACATTTTTAATGCTCCTTTCTT
and
(SEQ ID NO: 235)
TGATGAGGTGATTTTCGTGGA;
(vi)
(SEQ ID NO: 34)
TCTCTGTCCTCACAGAGTTCAAGC
and
(SEQ ID NO: 35)
CTTGGTCCAGCCAGGGACTA;
(vii)
(SEQ ID NO: 36)
CAAAAACATCCCACGCCTAGT
and
(SEQ ID NO: 37)
TGGGGTGAAGACCATTTTGAA;
(viii)
(SEQ ID NO: 238)
GCTGCAGTGGGACCACTATTATC;
and
(SEQ ID NO: 239)
GCTCCCGGAAGACAGTCCCC;
(ix)
(SEQ ID NO: 240)
CTGGCTGGACCAAGCCCATCA;
and
(SEQ ID NO: 241)
CTAGGCGAGGAGCTCCAGTCG;
(x)
(SEQ ID NO: 38)
GAGAAGAGGGTTGAGGAGTTCAA
and
(SEQ ID NO: 39)
GCAAAATCACATTATTGCCAACAT;
(xi)
(SEQ ID NO: 236)
GGCACGGTCTTCAGAGAAGC;
and
(SEQ ID NO: 237)
ACATTATTGCCAACATGACTTACTTGAT;
(xii)
(SEQ ID NO: 40)
CTAGTGCATTCAAGCACAATGG
and
(SEQ ID NO: 41)
CAGAGCCTAAACATCCCCTTAAA;
(xii)
(SEQ ID NO: 42)
AGGTGATCTATTTTTCCCTTTCTCC
and
(SEQ ID NO: 43)
TTATGTGTACCCAAAAAGGTGACA;
(xiv)
(SEQ ID NO: 44)
TGCCAGTTGTGCTTTTTGCT
and
(SEQ ID NO: 45)
ACAATAAAAGGCAGCTTGGACAC;
(xv)
(SEQ ID NO: 46)
ACTTGGCAGCCAGAAATATCCTC
and
(SEQ ID NO: 47)
GACTGTCAAGCAGAGAATGGGTACT;
(xvi)
(SEQ ID NO: 232)
GTATTCACAGAGACTTGGCAGC;
and
(SEQ ID NO: 233)
TGTCAAGCAGAGAATGGGTACT;
(xvii)
(SEQ ID NO: 48)
GATGTCTATGAAGTGTTGTGGTTCC
and
(SEQ ID NO: 49)
TGTTACAGAAATGAAATAAGACGGAAA;
(xviii)
(SEQ ID NO: 50)
GAGGCTCGCCATGCCGGGAG
and
(SEQ ID NO: 51)
GATGTCGATGGACGTCTCGTGC;
(xix)
(SEQ ID NO: 52)
CAGCGCCGCCTTCGGCTTTC
and
(SEQ ID NO: 53)
GCCCGGGTAGTCAAAGTCGC;
(xx)
(SEQ ID NO: 54)
GGCAGCAGGAGAAGGCCAAG
and
(SEQ ID NO: 55)
TTCATCCTCCTCGCGGGGCTC;
(xxi)
(SEQ ID NO: 56)
CGGCCGCTGGTGATCAAGC
and
(SEQ ID NO: 57)
GGTGACCGGGCTGCAGGTG;
(xxii)
(SEQ ID NO: 58)
CGCACTGCGGCCAGACCAC
and
(SEQ ID NO: 59)
GTTGCTGTTCTTGTCCACCGAC;
(xxiii)
(SEQ ID NO: 60)
GCGGGCAAGGCCAAGAAGTC
and
(SEQ ID NO: 61)
GTGTCCAGTTCGCGGCTCAG;
(xxiv)
(SEQ ID NO: 62)
GACCAGTGACAATGACCGCCTG
and
(SEQ ID NO: 63)
GGCGACCCCAAACCACTCC;

(xxv) a pair of primers having the reverse complement sequences of any of (i) through (xxiv); and
(xxvi) a pair of primers having at least 90% sequence identity to any of (i) through (xxv).

In another aspect, the disclosure relates to a kit comprising a pair of primers capable of amplifying a region of a FLT3 gene from a sample to produce a double-stranded oligonucleotide, wherein the pair of primers is selected from the group consisting of:

(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 32)
CTTGTCACCCACGGGAAAGT
and
(SEQ ID NO: 33)
AAAATAAGTAGGAAATAGCAGCCTCA;
(iv)
(SEQ ID NO: 234)
AGTTTTACATTTTTAATGCTCCTTTCTT
and
(SEQ ID NO: 235)
TGATGAGGTGATTTTCGTGGA;

(v) a pair of primers having the reverse complement sequences of any of (i) through (iv); and
(vi) a pair of primers having at least 90% sequence identity to any of (i) through (v).

In another aspect, the disclosure relates to a kit comprising a pair of primers capable of amplifying a region of a IDH2 gene from a sample to produce a double-stranded oligonucleotide, wherein the pair of primers is selected from the group consisting of:

(i)
(SEQ ID NO: 34)
TCTCTGTCCTCACAGAGTTCAAGC
and
(SEQ ID NO: 35)
CTTGGTCCAGCCAGGGACTA;
(ii)
(SEQ ID NO: 36)
CAAAAACATCCCACGCCTAGT
and
(SEQ ID NO: 37)
TGGGGTGAAGACCATTTTGAA;
(iii)
(SEQ ID NO: 238)
GCTGCAGTGGGACCACTATTATC;
and
(SEQ ID NO: 239)
GCTCCCGGAAGACAGTCCCC;
(iv)
(SEQ ID NO: 240)
CTGGCTGGACCAAGCCCATCA;
and
(SEQ ID NO: 241)
CTAGGCGAGGAGCTCCAGTCG;

(v) a pair of primers having the reverse complement sequences of any of (i) through (iv); and
(vi) a pair of primers having at least 90% sequence identity to any of (i) through (v).

In another aspect, the disclosure relates to a kit comprising a pair of primers capable of amplifying a region of a IDH1 gene from a sample to produce a double-stranded oligonucleotide, wherein the pair of primers is selected from the group consisting of:

(i)
(SEQ ID NO: 38)
GAGAAGAGGGTTGAGGAGTTCAA
and
(SEQ ID NO: 39)
GCAAAATCACATTATTGCCAACAT;
(ii)
(SEQ ID NO: 236)
GGCACGGTCTTCAGAGAAGC;
and
(SEQ ID NO: 237)
ACATTATTGCCAACATGACTTACTTGAT;

(iii) a pair of primers having the reverse complement sequences of any of (i) through (ii); and
(iv) a pair of primers having at least 90% sequence identity to any of (i) through (iii).

In another aspect, the disclosure relates to a kit comprising a pair of primers capable of amplifying a region of a KIT gene from a sample to produce a double-stranded oligonucleotide, wherein the pair of primers is selected from the group consisting of:

(i)
(SEQ ID NO: 40)
CTAGTGCATTCAAGCACAATGG
and
(SEQ ID NO: 41)
CAGAGCCTAAACATCCCCTTAAA;
(ii)
(SEQ ID NO: 42)
AGGTGATCTATTTTTCCCTTTCTCC
and
(SEQ ID NO: 43)
TTATGTGTACCCAAAAAGGTGACA;
(iii)
(SEQ ID NO: 44)
TGCCAGTTGTGCTTTTTGCT
and
(SEQ ID NO: 45)
ACAATAAAAGGCAGCTTGGACAC;
(v)
(SEQ ID NO: 232)
GTATTCACAGAGACTTGGCAGC;
and
(SEQ ID NO: 233)
TGTCAAGCAGAGAATGGGTACT;

(vi) a pair of primers having the reverse complement sequences of any of (i) through (v); and
(vii) a pair of primers having at least 90% sequence identity to any of (i) through (vi).

In another aspect, the disclosure relates to a kit comprising a pair of primers capable of amplifying a region of a NMP1 gene from a sample to produce a double-stranded oligonucleotide, wherein the pair of primers is selected from the group consisting of:

(i)
(SEQ ID NO: 48)
GATGTCTATGAAGTGTTGTGGTTCC
and
(SEQ ID NO: 49)
TGTTACAGAAATGAAATAAGACGGAAA;

(ii) a pair of primers having the reverse complement sequences of (i); and
(iii) a pair of primers having at least 90% sequence identity to any of (i) through (ii).

In another aspect, the disclosure relates to a kit comprising a pair of primers capable of amplifying a region of a CEBPA gene from a sample to produce a double-stranded oligonucleotide, wherein the pair of primers is selected from the group consisting of:

| | (i) |
|---|---|
| (SEQ ID NO: 50) | |
| | GAGGCTCGCCATGCCGGGAG |
| | and |
| (SEQ ID NO: 51) | |
| | GATGTCGATGGACGTCTCGTGC; |
| | (ii) |
| (SEQ ID NO: 52) | |
| | CAGCGCCGCCTTCGGCTTTC |
| | and |
| (SEQ ID NO: 53) | |
| | GCCCGGGTAGTCAAAGTCGC; |
| | (iii) |
| (SEQ ID NO: 54) | |
| | GGCAGCAGGAGAAGGCCAAG |
| | and |
| (SEQ ID NO: 55) | |
| | TTCATCCTCCTCGCGGGGCTC; |
| | (iv) |
| (SEQ ID NO: 56) | |
| | CGGCCGCTGGTGATCAAGC |
| | and |
| (SEQ ID NO: 57) | |
| | GGTGACCGGGCTGCAGGTG; |
| | (v) |
| (SEQ ID NO: 58) | |
| | CGCACTGCGGCCAGACCAC |
| | and |
| (SEQ ID NO: 59) | |
| | GTTGCTGTTCTTGTCCACCGAC; |
| | (vi) |
| (SEQ ID NO: 60) | |
| | GCGGGCAAGGCCAAGAAGTC |
| | and |
| (SEQ ID NO: 61) | |
| | GTGTCCAGTTCGCGGCTCAG; |
| | (vii) |
| (SEQ ID NO: 62) | |
| | GACCAGTGACAATGACCGCCTG |
| | and |
| (SEQ ID NO: 63) | |
| | GGCGACCCCAAACCACTCC; |

(vii) a pair of primers having the reverse complement sequences of any of (i) through (vii); and
(ix) a pair of primers having at least 90% sequence identity to any of (i) through (viii).

In another aspect, the disclosure relates to a kit comprising a pair of primers capable of amplifying a region of a gene of interest from a sample to produce a double-stranded oligonucleotide, wherein the pair of primers is selected from the group consisting of:

| | (i) |
|---|---|
| (SEQ ID NO: 64) | |
| | CCTTCCTCTTACTCTCTGCCTGC |
| | and |
| (SEQ ID NO: 65) | |
| | GACAGCGGGCACACTTACCAG; |
| | (ii) |
| (SEQ ID NO: 66) | |
| | CATTGTTAGGGCCGAGGCTAGA |
| | and |
| (SEQ ID NO: 67) | |
| | ACGCACTTGTTTTACCTGTATGAGTC; |
| | (iii) |
| (SEQ ID NO: 218) | |
| | GCCTCCCTTCCTCTTACTCTCT; |
| | and |
| | GCAGCCTGGGTAAGCACAC; |
| | (iv) |
| (SEQ ID NO: 68) | |
| | GCAGATTCAACTTTCACGTATCAAACC |
| | and |
| (SEQ ID NO: 69) | |
| | TGCAGAGCACGGGCTTTAATGT; |
| | (v) |
| (SEQ ID NO: 70) | |
| | CCTCGCAGACATTAAAGCCCGT |
| | and |
| (SEQ ID NO: 71) | |
| | CCACCATCACCACTGCTGCTG; |
| | (vi) |
| (SEQ ID NO: 72) | |
| | CAGTGGTGATGGTGGTGAGGC |
| | and |
| (SEQ ID NO: 73) | |
| | CCTAGCCCATCTGTGAGTCCAAC; |
| | (vii) |
| (SEQ ID NO: 74) | |
| | TGTCCTCCCAAACCTCAGTAGC |
| | and |
| (SEQ ID NO: 75) | |
| | AGCTTGGCCAGTTCCTTTCTCT; |
| | (viii) |
| (SEQ ID NO: 76) | |
| | GTGTCTCGAGTATGTGCGGTCC |
| | and |
| (SEQ ID NO: 77) | |
| | CATCGTGGGCTGGTGGAAGAA; |
| | (ix) |
| (SEQ ID NO: 192) | |
| | CTTTCACGTATCAAACCACCC |
| | and |
| (SEQ ID NO: 193) | |
| | CACGGGCTTTAATGTCTGC; |
| | (x) |
| (SEQ ID NO: 194) | |
| | AAGATCCCAGATTCCCTACTG |
| | and |
| (SEQ ID NO: 195) | |
| | GTGGGCTGGTGGAAGAAC; |
| | (xi) |
| (SEQ ID NO: 196) | |
| | GGCGAGAGGTCACCACTG |
| | and |
| (SEQ ID NO: 197) | |
| | CCATCACCACTGCTGCTG; |
| | (xii) |
| (SEQ ID NO: 198) | |
| | AACTGAATGTGAGTCTGGCA |
| | and |
| (SEQ ID NO: 199) | |
| | CACTAGAGACGGAATGGGAC; |
| | (xiii) |
| (SEQ ID NO: 200) | |
| | CAGTGGTGATGGTGGTGAG |
| | and |
| (SEQ ID NO: 201) | |
| | CCAACTGTAGCCCTCTGTAG; |
| | (xiv) |
| (SEQ ID NO: 78) | |
| | TCCATTGCCTCTCCTTCTGTGC |
| | and |
| (SEQ ID NO: 79) | |
| | TGCCAACTCCTTCATGCACCT; |
| | (xv) |
| (SEQ ID NO: 80) | |
| | CCTAGGGGATGTTCCAGATGGC |
| | and |
| (SEQ ID NO: 81) | |
| | AATGTGGGTTTGTTGCCATGAAAC; |
| | (xvi) |
| (SEQ ID NO: 82) | |
| | TCTATCGTGTCCCCACAGGGAA |
| | and |
| (SEQ ID NO: 83) | |
| | GGGGAAAGGTTGAACCCAAGGA; |
| | (xvii) |
| (SEQ ID NO: 84) | |
| | CTTCACGCCGCCTTCCACCG |
| | and |
| (SEQ ID NO: 85) | |
| | CTCCGGGCCAGTACCTTGAAA; |
| | (xviii) |
| (SEQ ID NO: 204) | |
| | AGCAACGCCCATTTCACC |
| | and |
| (SEQ ID NO: 205) | |
| | GCTCAGCTGCAAAGAATGTG; |
| | (xix) |
| (SEQ ID NO: 206) | |
| | CAGGCAAGATGAGCGAGGC |
| | and |
| (SEQ ID NO: 207) | |
| | GGGCCAGTACCTTGAAAGCG; |
| | (xx) |
| (SEQ ID NO: 86) | |
| | TCCTGCTGTGTGGTTAGACGG |
| | and |
| (SEQ ID NO: 87) | |
| | GAAGAGGTGGCGGATGACTGG; |
| | (xxi) |
| (SEQ ID NO: 202) | |
| | CTGCTGTGTGGTTAGACGG |
| | and |
| (SEQ ID NO: 203) | |
| | GAGGTGGCGGATGACTGG; |
| | (xxii) |
| (SEQ ID NO: 88) | |
| | GCCCTGGGCATTCCTTCTTTATT |
| | and |
| (SEQ ID NO: 89) | |
| | ACTTCTAAGATGTGGCAAGATGGC; |
| | (xxiii) |
| (SEQ ID NO: 90) | |
| | GTTGGGGCATAGTTAAAACCTGTGT |
| | and |
| (SEQ ID NO: 91) | |
| | TTCCTCTGTGTTGGCGGATACC; |
| | (xxiv) |
| (SEQ ID NO: 92) | |
| | GGTATCCGCCAACACAGAGGAA |
| | and |
| (SEQ ID NO: 93) | |
| | AGGAGACTGGAATTCTCGAATAAGGA; |
| | (XXV) |
| (SEQ ID NO: 208) | |
| | GGCTGCTGGTCTGGCTACTAT |
| | and |
| (SEQ ID NO: 209) | |
| | CCAGGGCAGAGGCTACAAC; |
| | (xxvi) |
| (SEQ ID NO: 210) | |
| | TGTAGGTCTTGTGGATGAGC |
| | and |
| (SEQ ID NO: 211) | |
| | TTCCTCTGTGTTGGCGGATA; |
| | (xxvii) |
| (SEQ ID NO: 212) | |
| | CAGAGGAAAGGTAAATCCACCA |
| | and |
| (SEQ ID NO: 213) | |
| | GGAATAAGATACCCAATAGCCTTCA; |
| | (xxviii) |
| (SEQ ID NO: 214) | |
| | TTCCTTCTTTATTGCCCTTCTTA |
| | and |
| (SEQ ID NO: 215) | |
| | CAACTTACCATGTTCAATGATTTCAA; |

(xxix) a pair of primers having the reverse complement sequences of any of (i) through (xxviii); and
(xxx) a pair of primers having at least 90% sequence identity to any of (i) through (xxix).

In another aspect, the disclosure relates to a kit comprising a pair of primers capable of amplifying a region of a WT1 gene from a sample to produce a double-stranded oligonucleotide, wherein the pair of primers is selected from the group consisting of:

| | (i) |
|---|---|
| (SEQ ID NO: 218) | |
| | GCCTCCCTTCCTCTTACTCTCT; |
| | and |
| | GCAGCCTGGGTAAGCACAC; |

(ii) a pair of primers having the reverse complement sequences of (i); and
(iii) a pair of primers having at least 90% sequence identity to any of (i) through (ii).

In another aspect, the disclosure relates to a kit comprising a pair of primers capable of amplifying a region of a ASXL1 gene from a sample to produce a double-stranded oligonucleotide, wherein the pair of primers is selected from the group consisting of:

| | (i) |
|---|---|
| (SEQ ID NO: 192) | |
| | CTTTCACGTATCAAACCACCC |
| | and |
| (SEQ ID NO: 193) | |
| | CACGGGCTTTAATGTCTGC; |
| | (ii) |
| (SEQ ID NO: 194) | |
| | AAGATCCCAGATTCCCTACTG |
| | and |
| (SEQ ID NO: 195) | |
| | GTGGGCTGGTGGAAGAAC; |
| | (iii) |
| (SEQ ID NO: 196) | |
| | GGCGAGAGGTCACCACTG |
| | and |
| (SEQ ID NO: 197) | |
| | CCATCACCACTGCTGCTG; |
| | (iv) |
| (SEQ ID NO: 198) | |
| | AACTGAATGTGAGTCTGGCA |
| | and |
| (SEQ ID NO: 199) | |
| | CACTAGAGACGGAATGGGAC; |
| | (v) |
| (SEQ ID NO: 200) | |
| | CAGTGGTGATGGTGGTGAG |
| | and |
| (SEQ ID NO: 201) | |
| | CCAACTGTAGCCCTCTGTAG; |

(vi) a pair of primers having the reverse complement sequences of any of (i) through (v); and
(vii) a pair of primers having at least 90% sequence identity to any of (i) through (vi).

In another aspect, the disclosure relates to a kit comprising a pair of primers capable of amplifying a region of a RUNX1 gene from a sample to produce a double-stranded oligonucleotide, wherein the pair of primers is selected from the group consisting of:

| | (i) |
|---|---|
| (SEQ ID NO: 204) | |
| | AGCAACGCCCATTTCACC |
| | and |
| (SEQ ID NO: 205) | |
| | GCTCAGCTGCAAAGAATGTG; |
| | (ii) |
| (SEQ ID NO: 206) | |
| | CAGGCAAGATGAGCGAGGC |
| | and |
| (SEQ ID NO: 207) | |
| | GGGCCAGTACCTTGAAAGCG; |

(iii) a pair of primers having the reverse complement sequences of any of (i) through (ii); and
(iv) a pair of primers having at least 90% sequence identity to any of (i) through (iii).

In another aspect, the disclosure relates to a kit comprising a pair of primers capable of amplifying a region of a DNMT3A gene from a sample to produce a double-stranded oligonucleotide, wherein the pair of primers is selected from the group consisting of:

| | (i) |
|---|---|
| (SEQ ID NO: 202) | |
| | CTGCTGTGTGGTTAGACGG |
| | and |
| (SEQ ID NO: 203) | |
| | GAGGTGGCGGATGACTGG; |

(ii) a pair of primers having the reverse complement sequences of (i); and
(iii) a pair of primers having at least 90% sequence identity to any of (i) through (ii).

In another aspect, the disclosure relates to a kit comprising a pair of primers capable of amplifying a region of a SFB31 gene from a sample to produce a double-stranded oligonucleotide, wherein the pair of primers is selected from the group consisting of:

| | (i) |
|---|---|
| (SEQ ID NO: 208) | |
| | GGCTGCTGGTCTGGCTACTAT |
| | and |
| (SEQ ID NO: 209) | |
| | CCAGGGCAGAGGCTACAAC; |
| | (ii) |
| (SEQ ID NO: 210) | |
| | TGTAGGTCTTGTGGATGAGC |
| | and |
| (SEQ ID NO: 211) | |
| | TTCCTCTGTGTTGGCGGATA; |
| | (iii) |
| (SEQ ID NO: 212) | |
| | CAGAGGAAAGGTAAATCCACCA |
| | and |
| (SEQ ID NO: 213) | |
| | GGAATAAGATACCCAATAGCCTTCA; |
| | (iv) |
| (SEQ ID NO: 214) | |
| | TTCCTTCTTTATTGCCCTTCTTA |
| | and |
| (SEQ ID NO: 215) | |
| | CAACTTACCATGTTCAATGATTTCAA; |

(v) a pair of primers having the reverse complement sequences of any of (i) through (iv); and
(vi) a pair of primers having at least 90% sequence identity to any of (i) through (v).

In another aspect, the disclosure relates to a kit comprising a pair of primers capable of amplifying a region of a gene of interest from a sample to produce a double-stranded oligonucleotide, wherein the pair of primers is selected from the group consisting of:

| | (i) |
|---|---|
| (SEQ ID NO: 88) | |
| | GCCCTGGGCATTCCTTCTTTATT |
| | and |
| (SEQ ID NO: 89) | |
| | ACTTCTAAGATGTGGCAAGATGGC; |
| | (ii) |
| (SEQ ID NO: 90) | |
| | GTTGGGGCATAGTTAAAACCTGTGT |
| | and |
| (SEQ ID NO: 91) | |
| | TTCCTCTGTGTTGGCGGATACC; |
| | (iii) |
| (SEQ ID NO: 92) | |
| | GGTATCCGCCAACACAGAGGAA |
| | and |
| (SEQ ID NO: 93) | |
| | AGGAGACTGGAATTCTCGAATAAGGA; |
| | (iv) |
| (SEQ ID NO: 208) | |
| | GGCTGCTGGTCTGGCTACTAT |
| | and |
| (SEQ ID NO: 209) | |
| | CCAGGGCAGAGGCTACAAC; |
| | (v) |
| (SEQ ID NO: 210) | |
| | TGTAGGTCTTGTGGATGAGC |
| | and |
| (SEQ ID NO: 211) | |
| | TTCCTCTGTGTTGGCGGATA; |
| | (vi) |
| (SEQ ID NO: 212) | |
| | CAGAGGAAAGGTAAATCCACCA |
| | and |
| (SEQ ID NO: 213) | |
| | GGAATAAGATACCCAATAGCCTTCA; |
| | (vii) |
| (SEQ ID NO: 214) | |
| | TTCCTTCTTTATTGCCCTTCTTA |
| | and |
| (SEQ ID NO: 215) | |
| | CAACTTACCATGTTCAATGATTTCAA; |
| | (viii) |
| (SEQ ID NO: 230) | |
| | CTACTCCTCGCCTGTGGACAAC |
| | and |
| (SEQ ID NO: 231) | |
| | GATCTGGGACTGCATGCTGGTG; |
| | (ix) |
| (SEQ ID NO: 220) | |
| | CCAACGTCAGTGAGGCAGAT |
| | and |
| (SEQ ID NO: 221) | |
| | GGATGACAATACCAGGCAGGA; |
| | (x) |
| (SEQ ID NO: 222) | |
| | TGCTTTCCTTGGAGCCAAAT |
| | and |
| (SEQ ID NO: 223) | |
| | TGTGTTAGCTGGAGTGAAAACTTG; |
| | (xi) |
| (SEQ ID NO: 224) | |
| | ATATGCCTGAGCGTTTCGATGC |
| | and |
| (SEQ ID NO: 225) | |
| | GTGGCCTTCTAGCCAACCTCT; |
| | (xii) |
| (SEQ ID NO: 226) | |
| | CCCAGGGGATATGCTGAACTA |
| | and |
| (SEQ ID NO: 227) | |
| | ACCTGGAGAGAGGCTGAGTG; |
| | (xiii) |
| (SEQ ID NO: 228) | |
| | TTGGCTTGCAGGTGCCCATC |
| | and |
| (SEQ ID NO: 229) | |
| | GGCGAGTCCAGAACCAAGATTT; |

(xiv) a pair of primers having the reverse complement sequences of any of (i) through (xiii); and
(xv) a pair of primers having at least 90% sequence identity to any of (i) through (xiv).

In another aspect, the disclosure relates to a kit comprising a pair of primers capable of amplifying a region of a SFB31 gene from a sample to produce a double-stranded oligonucleotide, wherein the pair of primers is selected from the group consisting of:

| | (i) |
|---|---|
| (SEQ ID NO: 208) | |
| | GGCTGCTGGTCTGGCTACTAT |
| | and |
| (SEQ ID NO: 209) | |
| | CCAGGGCAGAGGCTACAAC; |
| | (ii) |
| (SEQ ID NO: 210) | |
| | TGTAGGTCTTGTGGATGAGC |
| | and |
| (SEQ ID NO: 211) | |
| | TTCCTCTGTGTTGGCGGATA; |
| | (iii) |
| (SEQ ID NO: 212) | |
| | CAGAGGAAAGGTAAATCCACCA |
| | and |
| (SEQ ID NO: 213) | |
| | GGAATAAGATACCCAATAGCCTTCA; |
| | (iv) |
| (SEQ ID NO: 214) | |
| | TTCCTTCTTTATTGCCCTTCTTA |
| | and |
| (SEQ ID NO: 215) | |
| | CAACTTACCATGTTCAATGATTTCAA; |

(v) a pair of primers having the reverse complement sequences of any of (i) through (iv); and
(vi) a pair of primers having at least 90% sequence identity to any of (i) through (v).

In another aspect, the disclosure relates to a kit comprising a pair of primers capable of amplifying a region of a NOTCH1 gene from a sample to produce a double-stranded oligonucleotide, wherein the pair of primers is selected from the group consisting of:

| | (i) |
|---|---|
| (SEQ ID NO: 230) | |
| | CTACTCCTCGCCTGTGGACAAC |
| | and |
| (SEQ ID NO: 231) | |
| | GATCTGGGACTGCATGCTGGTG; |

(ii) a pair of primers having the reverse complement sequences of (i); and
(iii) a pair of primers having at least 90% sequence identity to any of (i) through (ii).

In another aspect, the disclosure relates to a kit comprising a pair of primers capable of amplifying a region of a CXCR4 gene from a sample to produce a double-stranded oligonucleotide, wherein the pair of primers is selected from the group consisting of:

| | (i) |
|---|---|
| (SEQ ID NO: 220) | |
| | CCAACGTCAGTGAGGCAGAT |
| | and |
| (SEQ ID NO: 221) | |
| | GGATGACAATACCAGGCAGGA; |
| | (ii) |
| (SEQ ID NO: 222) | |
| | TGCTTTCCTTGGAGCCAAAT |
| | and |
| (SEQ ID NO: 223) | |
| | TGTGTTAGCTGGAGTGAAAACTTG; |

(iii) a pair of primers having the reverse complement sequences of any of (i) through (ii); and
(iv) a pair of primers having at least 90% sequence identity to any of (i) through (iii).

In another aspect, the disclosure relates to a kit comprising a pair of primers capable of amplifying a region of a MYD88 gene from a sample to produce a double-stranded oligonucleotide, wherein the pair of primers is selected from the group consisting of:

| | (i) |
|---|---|
| (SEQ ID NO: 224) | |
| | ATATGCCTGAGCGTTTCGATGC |
| | and |
| (SEQ ID NO: 225) | |
| | GTGGCCTTCTAGCCAACCTCT; |
| | (ii) |
| (SEQ ID NO: 226) | |
| | CCCAGGGGATATGCTGAACTA |
| | and |
| (SEQ ID NO: 227) | |
| | ACCTGGAGAGAGGCTGAGTG; |
| | (iii) |
| (SEQ ID NO: 228) | |
| | TTGGCTTGCAGGTGCCCATC |
| | and |
| (SEQ ID NO: 229) | |
| | GGCGAGTCCAGAACCAAGATTT; |

(iv) a pair of primers having the reverse complement sequences of any of (i) through (iii); and
(v) a pair of primers having at least 90% sequence identity to any of (i) through (iv).

In another aspect, the disclosure relates to a kit comprising two or more primers capable of amplifying a region of a gene of interest from a sample to produce a double-stranded oligonucleotide, wherein the pair of primers is selected from the group consisting of:

| | (i) |
|---|---|
| (SEQ ID NO: 243) | |
| | ATGCTGACCAACTCGTGTGTGAA; |
| | (ii) |
| (SEQ ID NO: 244) | |
| | CAGAACTCGCAACAGTCCTTCGA; |
| | (iii) |
| (SEQ ID NO: 245) | |
| | GTGCGTGGAGGAGATCGAGCG; |
| | (iv) |
| (SEQ ID NO: 246) | |
| | TGATTATAGCCTAAGACCCGGAGC; |
| | (v) |
| (SEQ ID NO: 247) | |
| | CCTAAGACCCGGAGCTTTTCAC; |

(vi) two or more primers having the reverse complement sequences of any of (i) through (v); and
(vii) two or more primers having at least 90% sequence identity to any of (i) through (vi).

In another aspect, the disclosure relates to a kit comprising a pair of primers capable of amplifying a region of a BCR/ABL1 fusion gene from a sample to produce a double-stranded oligonucleotide, wherein the two or more primers are selected from the group consisting of:

| | (i) |
|---|---|
| (SEQ ID NO: 243) | |
| | ATGCTGACCAACTCGTGTGTGAA; |
| | (iii) |
| (SEQ ID NO: 246) | |
| | TGATTATAGCCTAAGACCCGGAGC; |
| | (iii) |
| (SEQ ID NO: 247) | |
| | CCTAAGACCCGGAGCTTTTCAC; |

(iv) two or more primers having the reverse complement sequences of any of (i) through (iii); and
(v) two or more primers having at least 90% sequence identity to any of (i) through (iv).

In another aspect, the disclosure relates to a kit comprising a pair of primers capable of amplifying a region of a BCR/ABL1 fusion gene from a sample to produce a double-stranded oligonucleotide, wherein the two or more primers are selected from the group consisting of:

| | (i) |
|---|---|
| (SEQ ID NO: 244) | |
| | CAGAACTCGCAACAGTCCTTCGA; |
| | (iii) |
| (SEQ ID NO: 246) | |
| | TGATTATAGCCTAAGACCCGGAGC; |
| | (iii) |
| (SEQ ID NO: 247) | |
| | CCTAAGACCCGGAGCTTTTCAC; |

(iv) two or more primers having the reverse complement sequences of any of (i) through (iii); and
(v) two or more primers having at least 90% sequence identity to any of (i) through (iv).

In another aspect, the disclosure relates to a kit comprising a pair of primers capable of amplifying a region of a BCR/ABL1 fusion gene from a sample to produce a double-stranded oligonucleotide, wherein the two or more primers are selected from the group consisting of:

(i)
(SEQ ID NO: 245)
GTGCGTGGAGGAGATCGAGCG;
(ii)
(SEQ ID NO: 246)
TGATTATAGCCTAAGACCCGGAGC;
(iii)
(SEQ ID NO: 247)
CCTAAGACCCGGAGCTTTTCAC;

(iv) two or more primers having the reverse complement sequences of any of (i) through (iii); and
(v) two or more primers having at least 90% sequence identity to any of (i) through (iv).

The kit can also include one or more reagents suitable for performing High Resolution Melting (HRM) analysis.

In certain embodiments, the kit can include at least 2, 3, or 4 pairs of primers from (i) through (vii). In certain embodiments, the kit can include at least 2, 3, or 4 pairs of primers from (i) through (xxvi). In certain embodiments, the kit can include at least 2, 3, or 4 pairs of primers from (i) through (xxx). In certain embodiments, the kit can include at least 2, 3, or 4 pairs of primers from (i) through (xv). In certain embodiments, the amplifying step comprises performing PCR using, in parallel, at least 2, 3, or 4 pairs of primers from (i) through (v).

In certain embodiments, the kit can further comprise a wild-type or mutant (i.e., positive) control. In certain embodiments, the wild-type control is wild-type DNA from the region of the gene of interest. In certain embodiments, the mutant control is DNA comprising a mutation in the region of the gene of interest.

The kit may also include a dye, such as SYTO® 9, SYBR® Green, or Chai Green™.

In another aspect, the disclosure relates to a method for performing a high resolution melting analysis (HRM) to detect the presence or absence of a mutation in a nucleic acid, the method comprising:
(a) combining the nucleic acid, at least one primer pair, a dye, and a reaction mixture comprising 10 mM Tris-HCl, 50 mM KCl, 1.5 mM MgCl₂, 0.2 mM dNTPs, 5% Glycerol, and 25 units/mL of a DNA polymerase;
(b) amplifying the nucleic acid; and
(c) detecting the presence or absence of the mutation by performing a melting curve analysis of the amplified product.

In another aspect, the disclosure relates to a method for performing a high resolution melting analysis (HRM) to detect the presence or absence of a mutation in a nucleic acid, the method comprising:
(a) combining the nucleic acid, at least one primer pair, a DNA polymerase, dNTPs, optionally dUTPs, and a dye;
(b) amplifying the nucleic acid; and
(c) detecting the presence or absence of the mutation by performing a melting curve analysis of the amplified product.

In certain embodiments, the DNA polymerase comprises Hot Start Taq DNA polymerase. In certain embodiments, the DNA polymerase comprises AmpliTaq Gold™ 360 DNA Polymerase. In certain embodiments, the dye comprises MeltDoctor™ HRM Dye. In certain embodiments, the dye comprises SYTO® 9, SYBR® Green, or Chai Green™. In certain embodiments, the dye comprises MeltDoctor™ HRM Dye.

In certain embodiments, the method further comprises the step of sequencing the mutation. In certain embodiments, the detecting step comprises comparing the HRM analysis results of the nucleic acid to a control. In certain embodiments, the control comprises HRM analysis results of a wild-type sample. In certain embodiments, the control comprises HRM analysis results of a sample comprising the mutation.

The description above describes multiple aspects and embodiments of the invention. The patent application specifically contemplates all combinations and permutations of the aspects and embodiments. These and other aspects and features of the invention are described in the following detailed description and claims.

### BRIEF DESCRIPTION OF THE DRAWINGS

FIG. 1 is a schematic diagram of three assays to detect exons 1, 13, or 19 of the BCR gene as well as exon 3 of the ABL gene to detect one the p210, p190, p230, or p203 isoforms of a BCR/ABL1 fusion gene. The variant isoforms include variants of isoforms p210 and p203 between BCR exons 13 and 14 (b2 and b3) and ABL exons 2 and 3 (a2 and a3), two variants of isoform p190 between BCR exons 1 and ABL exons 1 and 2 (e1 and a2) and exon 3 (a3), and two variants of isoform p230 between BCR exon 19 (e19) and ABL exons 2 and 3 (a2 and a3) Arrows depict the binding of primers.

FIGS. 2A-2H are a set of graphs showing exemplary melt curve signatures for the BCR/ABL1 isoforms and three assays described in FIG. 1. FIGS. 2A-2H show an exemplary melting curve for an amplicon of isoform b2a2 (e13a2), b3a2 (e14a2), b2a3 (e13a3), b3a3 (e14a3), e1a2, e1a3, e19a2, and e19a3, respectively. These melting curve show a peak at about 75° C. for an internal amplification control and at >80° C. for an amplicon containing a fusion product.

### DETAILED DESCRIPTION

The present disclosure is based in part, upon the discovery of a rapid, low-cost method of detecting the presence or absence of a mutation in one or more of the genes JAK2 (Janus Kinase 2), MPL (thrombopoietin receptor), and CALR (calreticulin), which are associated with myeloid malignancies.

In certain embodiments, the mutation is in exon 10 of the MPL gene, in exon 9 of the CALR gene, or in exon 12, 13 or 14 of the JAK2 gene. The method may include amplifying (e.g., by PCR) a portion of one or more of the exons using the primers set forth in SEQ ID NOs: 1 and 2, SEQ ID NOs: 3 and 4, SEQ ID NOs: 5 and 6, SEQ ID NOs: 7 and 8, or SEQ ID NOs: 21 and 22, or primers having the reverse complement sequence to any of the foregoing, or primers having 90% sequence identity to any of the foregoing, such that the primer pair can amplify at least a portion of the sequence provided in one or more of SEQ ID NOs: 9-20 or SEQ ID NOs: 23-25. In certain embodiments, the method includes the use of high resolution melting (HRM) to detect the presence or absence of the mutation.

In certain embodiments, the methods relate to detecting the following mutations in parallel: MPL W515L (c.1544G>T), CALR L367fs*46 (c.1092_1143del52), JAK2 V617F (c.1849G>T), and JAK2 N542_E543del (c.1624_1629delAATGAA). In certain embodiments, the method includes amplifying (e.g., by PCR) a portion of one or more of the exons using the primers set forth in SEQ ID NOs: 1 and 2, SEQ ID NOs: 3 and 4, SEQ ID NOs: 5 and 6, SEQ ID NOs: 7 and 8, or SEQ ID NOs: 21 and 22, or primers having the reverse complement sequence to any of the foregoing, or primers having 90% sequence identity to any of the foregoing, such that the primer pair can amplify at least a portion of the MPL gene exon 10, the sequence of which is provided in RefSeq Number NC_000001.11, the CALR gene exon 9, the sequence of which is provided in RefSeq Number NC_000019.10, and/or the JAK2 gene exons 12, 13, or 14, the sequences of which are provided in RefSeq Number NC_000009.12, and using high resolution melting (HRM) to detect the presence or absence of the mutations.

The present disclosure also is based in part, upon the discovery of a rapid, low-cost method of detecting the presence or absence of a mutation in one or more of the genes FLT3, IDH2, IDH1, KIT, NPM1, and/or CEBPA, which are associated with acute myeloid leukemia.

In certain embodiments, the methods relate to detecting the 2, 3, 4, 5, 6, 7, 8, 9, 10, 11, 12, 13, 14, 15, 16, 17, 18, or 19 of the following mutations in parallel: internal tandem duplications in exons 14, 15, or 16 of FLT3 or mutations in codons 835 and 836 of exon 20 of FLT3: IDH2 c.418C>G (p.R140G): IDH2 c.418C>T (p.R140W): IDH2 c.419G>T (p.R140L): IDH2 c.419G>A (p.R140Q): IDH2 c.514A>T (p.R172W): IDH2 c.515G>A (p.R172K): IDH2 c.515G>T (p.R172M): IDH2 c.516G>C (p.R172S): IDH2 c.516G>T (p.R172S): IDH1 c.298C>T (p.R100*): IDH1 c.299G>A (p.R100Q): IDH1 c.313G>C (p.G105R): IDH1 c.314G>T (p.G105V): IDH1 c.314G>A (p.G105D): IDH1 c.394C>A (p.R132S): IDH1 c.394C>T (p.R132C): IDH1 c.394C>G (p.R132G): IDH1 c.395G>A (p.R132H): IDH1 c.395G>T (p.R132L)′IDH1 c.395G>C (p.R132P): KIT 1504_1509dup (p.A502_Y503dup); KIT c.1669_1674del (p.W557_K558del); KIT c. 1669_1683del (p.W557_E561del): KIT c. 1669T>G (p.W557G): KIT c.1669T>C (p.W557R): KIT c. 1669T>A (p.W557R): KIT c.1676T>C (p.V559A): KIT c.1676T>A (p.V559D): KIT c. 1676T>G (p.V559G): KIT c. 1679T>A (p.V560D): KIT c. 1727T>C (p.L576P): KIT c. 1924A>G (p.K642E): KIT c. 1961T>C (p.V654A): KIT c.1965T>A (p.N655K): KIT c.2446G>C (p.D816H): KIT c.2466T>A (p.N822K): KIT c.2446G>T (p.D816Y): KIT c.2446_2447GA>AT (p.D816I): KIT c.2447A>T (p.D816V): KIT c.2458G>T (p.D820Y): KIT c.2459A>G (p.D820G): KIT c.2464A>T (p.N822Y): KIT c.2466T>G (p.N822K): KIT c.2467T>G (p.Y823D): KIT c.2474T>C (p.V825A): NPM1 c.860_863dup (p.W288Cfs*12): one or more mutations in CEBPA.

The method may include amplifying (e.g., by PCR) a portion of one or more of the exons using the primers set forth in SEQ ID NOs: 26 and 27, SEQ ID NOs: 28 and 29, SEQ ID NOs: 30 and 31, SEQ ID NOs: 32 and 33, SEQ ID NOs: 234 and 235, SEQ ID NOs: 34 and 35, SEQ ID NOs: 36 and 37, SEQ ID NOs: 238 and 239, SEQ ID NOs: 240 and 241, SEQ ID NOs: 38 and 39, SEQ ID NOs: 236 and 237, SEQ ID NOs: 40 and 41, SEQ ID NOs: 42 and 43, SEQ ID NOs: 44 and 45, SEQ ID NOs: 46 and 47, SEQ ID NOs: 232 and 233, SEQ ID NOs: 48 and 49, SEQ ID NOs: 50 and 51, SEQ ID NOs: 52 and 53, SEQ ID NOs: 54 and 55, SEQ ID NOs: 56 and 57, SEQ ID NOs: 58 and 59, SEQ ID NOs: 60 and 61, or SEQ ID NOs: 62 and 63, or primers having the reverse complement sequence to any of the foregoing, or primers having 90% sequence identity to any of the foregoing, such that the primer pair can amplify at least a portion of the FLT3 gene exons 14, 15, 16, and 20, the sequences of which are provided in RefSeq Number NC_000009.12, the IDH2 gene exon 4, the sequence of which is provided in RefSeq Number NC_000015.10, the IDH1 gene exon 4, the sequence of which is provided in RefSeq Number NC_000002.12, the KIT gene exons 9, 11, 13, and 17, the sequences of which are provided in RefSeq Number NC_000004.12, the NPM1 gene exon 12, the sequence of which is provided in RefSeq Number NC_000005.10, and/or the CEBPA gene exon 1, the sequence of which is provided in RefSeq Number NC_000019.10. In certain embodiments, the method includes the use of high resolution melting (HRM) to detect the presence or absence of the mutation.

The present disclosure is based in part, upon the discovery of a rapid, low-cost method of detecting the presence or absence of a mutation in one or more of the genes WT1, ASXL1, RUNX1, DNMT3, SF3B1, and/or TERT which are associated with myeloid malignancies.

In certain embodiments, the methods relate to detecting the 2, 3, 4, 5, 6, 7, 8, 9, 10, 11, 12, 13, 14, 15, 16, 17, or 18 of the following mutations in parallel: WT1 c.1110dup (p.V371Cfs*14): WT1 c.1142C>A (p.S381*): WT1 c.1384C>T (p.R462W): WT1 c.1385G>A (p.R462Q): WT1 c.1385G>C (p.R462P): WT1 c.1390G>A (p.D464N): ASXL1 Exon 12 codon 591: c. 1772dup (p.Y591*): ASXL1 Exon 12 codon 635: c. 1900_1922del (p.E635Rfs*15); ASXL1 Exon 12 codon 646: c. 1934dup (p.G646Wfs*12): ASXL1 Exon 12 codon 693:
c.2077C>T (p.R693*): ASXL1 Exon 12 codon 808: c.2423del (p.P808Lfs*10): ASXL1 Exon 12 codon 1102: c.3306G>T (p.E1102D): RUNX1 c.167T>C (p.L56S): RUNX1 c.319C>T (p.R107C): RUNX1 c.422C>A (p.S141*): RUNX1 c.485G>A (p.R162K): RUNX1 c.496C>T (p.R166*): RUNX1 c.592G>A (p.D198N): RUNX1 c.602G>A (p.R201Q): RUNX1 c.610C>T (p.R204*): RUNX1 c.958C>T (p.R320*); DNMT3A c.2644C>G (p.R882G): DNMT3A c.2644C>T (p.R882C): DNMT3A c.2644C>A (p.R882S): DNMT3A c.2645G>A (p.R882H); DNMT3A c.2645G>C (p.R882P): DNMT3A c.2645G>T (p.R882L); and one or more mutations in SF2B1.

The method may include amplifying (e.g., by PCR) a portion of one or more of the exons using the primers set forth in SEQ ID NOs: 64 and 65, SEQ ID NOs: 66 and 67, SEQ ID NOs: 216 and 217, SEQ ID NOs: 218 and 219, SEQ ID NOs: 68 and 69, SEQ ID NOs: 70 and 71, SEQ ID NOs: 72 and 73, SEQ ID NOs: 74 and 75, SEQ ID NOs: 76 and 77, SEQ ID NOs: 192 and 193, SEQ ID NOs: 194 and 195, SEQ ID NOs: 196 and 197, SEQ ID NOs: 198 and 199, SEQ ID NOs: 200 and 201SEQ ID NOs: 78 and 79, SEQ ID NOs: 80 and 81, SEQ ID NOs: 82 and 83, SEQ ID NOs: 84 and 85, SEQ ID NOs: 204 and 205, SEQ ID NOs: 206 and 207, SEQ ID NOs: 86 and 87, SEQ ID NOs: 202 and 203, SEQ ID NOs: 88 and 89, SEQ ID NOs: 90 and 91, SEQ ID NOs: 92 and 93, SEQ ID NOs: 208 and 209, SEQ ID NOs: 210 and 211, SEQ ID NOs: 212 and 213, SEQ ID NOs: 214 and 215, or primers having the reverse complement sequence to any of the foregoing, or primers having 90% sequence identity to any of the foregoing, such that the primer pair can amplify at least a portion of the WT1 gene exons 8 and 10, the sequences of which are provided in RefSeq Number NC_000011.10, the ASXL1 gene exon 12, the sequence of which is provided in RefSeq Number NC_000020.11, the RUNX1 gene exons 4, 5, 6, and 8, the sequences of which are provided in RefSeq Number NC_000021.9, the DNMT3A gene exon 23, the sequence of which is provided in RefSeq Number NC_000002.12, the TERT promoter and/or exon 5, the sequences of which are provided in RefSeq Number NC_000005.10, and/or the SF3B1 gene exons 15, 16, and 17, the sequences of which are provided in RefSeq Number NC_000002.12. In certain embodiments, the method includes the use of high resolution melting (HRM) to detect the presence or absence of the mutation.

The present disclosure is based in part, upon the discovery of a rapid, low-cost method of detecting the presence or absence of a mutation in one or more of the genes SF3B1, NOTCH1, CXCR4, and/or MYD88 which are associated with myeloid malignancies.

In certain embodiments, the methods relate to detecting the 2, 3, 4, 5, 6, 7, 8, 9, 10, 11, 12, 13, 14, 15, 16, 17, or 18 of the following mutations in parallel: SFB31 c.1866G>T; p.E622D, SFB31 c.1866G>C; p.E622D, SFB31 c.1873C>T; p.R625C, SFB31 c.1874G>T; p.R625L, SFB31 c.1984C>G; p.H662D, SFB31 c.1986C>G; p.H662Q, SFB31 c.1986C>A; p.H662Q, SFB31 c.1996A>C; p.K666Q, SFB31 c.1996A>G; p.K666E, SFB31 c. 1997A>C; p.K666T, SFB31 c.1997A>G; p.K666R, SFB31 c.1998G>T; p.K666N, SFB31 or c. 1998G>C; p.K666N; Exon 16, SFB31 c.2098A>G; p.K700E; or Exon 17 c.2225G>A; p.G742D, Notch 1 c.7541_7542delCT; p.P2514Rfs*4, CXCR3 c.598C>T; p.Q200*, CXCR3 c.952dup; p.T318Nfs*26, CXCR3 c.959_960del; p. V320Efs*23 c.993dup; p.G332Rfs*12, CXCR3 c.997A>T; p.K333*, CXCR3 c.1000C>T; p.R334*, CXCR3 c.1005dup; p.G336Wfs*8, CXCR3 c. 1012_1015del; p.S338Lfs*27 c.1012dup; p.S338Ffs*6, CXCR3 c.1013C>A; p.S338*, CXCR3 c. 1013C>G; p.S338, CXCR3 c. 1014_1017del; p.S339Ffs*26, CXCR3 or c. 1021del; p.S341Pfs*25, MYD88 c.649G>T; p.V217F MYD88 c.656C>G; p.S219C; MYD88 c.695T>C; p.M232T MYD88 c.728G>A; p.S243N; and MYD88 5 c.794T>C; p.L265P.

The method may include amplifying (e.g., by PCR) a portion of one or more of the exons using the primers set forth in 88 and 89, SEQ ID NOs: 90 and 91, SEQ ID NOs: 92 and 93, SEQ ID NOs: 208 and 209, SEQ ID NOs: 210 and 211, SEQ ID NOs: 212 and 213, SEQ ID NOs: 214 and 215, SEQ ID NOs: 220 and 221, SEQ ID NOs: 222 and 223, SEQ ID NOs: 224 and 225, SEQ ID NOs: 226 and 227, SEQ ID NOs: 228 and 229, and SEQ ID NOs: 230 and 231, or primers having the reverse complement sequence to any of the foregoing, or primers having 90% sequence identity to any of the foregoing, such that the primer pair can amplify at least a portion of the SF3B1 gene exons 15, 16, and 17, the sequences of which are provided in RefSeq Number NC_000002.12, the NOTCH1 gene exon 34, the sequence of which is provided in RefSeq Number NC_000009.12, the CXCR4 gene exon 2, the sequence of which is provided in RefSeq Number NC_000002.12, and/or the MYD88 gene exons 3, 4, and 5, the sequences of which are provided in RefSeq Number NC_000003.12. In certain embodiments, the method includes the use of high resolution melting (HRM) to detect the presence or absence of the mutation.

The present disclosure also is based in part, upon the discovery of a rapid, low-cost method of detecting the presence or absence of an exon in one or more of the genes BCR and/or ABL1, which are associated with BCR/ABL1-induced leukemogenesis (e.g., chronic myeloid leukemia (CML) or acute lymphocytic leukemia (ALL)).

In certain embodiments, the methods relate to detecting the presence of the following exons in parallel: BCR exon 13 and ABL1 exon 3, BCR exon 1 and ABL1 exon 3, or BCR exon 19 and ABL1 exon 3.

The method may include amplifying (e.g., by PCR) a portion of one or more of the exons using two or more of the primers set forth in SEQ ID NOs: 243-247, or primers having the reverse complement sequence to any of the foregoing, or primers having 90% sequence identity to any of the foregoing, such that the primer pair can amplify at least a portion of the BCR gene exons 1, 13, and 19 and/or the ABL1 gene exon 3. In certain embodiments, the method includes the use of high resolution melting (HRM) to detect the presence or absence of the mutation.

The methods described herein are advantageous, in part, because they allow a clinician to quickly and inexpensively (through use of HRM in contrast to direct sequencing), rule out the presence of mutations in these genes. Only when a sample tests positive for a mutation is the slower, more expensive step of sequencing needed to confirm the presence of the mutation.

### I. Definitions

The following definitions are included for the purpose of understanding the present subject matter and for constructing the appended patent claims.

Throughout the application, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

It should be understood that the expression “at least one of” includes individually each of the recited objects after the expression and the various combinations of two or more of the recited objects unless otherwise understood from the context and use.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

The use of the singular herein, for example, “a,” “an,” or “the,” includes the plural (and vice versa) unless specifically stated otherwise.

Where the use of the term “about” is before a quantitative value, the present teachings also include the specific quantitative value itself, unless specifically stated otherwise. As used herein, the term “about” refers to a ±15% variation from the nominal value unless otherwise indicated or inferred.

It should be understood that the order of steps or order for performing certain actions is immaterial so long as the present teachings remain operable. Moreover, two or more steps or actions can be conducted simultaneously.

At various places in the present specification, values are disclosed in groups or in ranges. It is specifically intended that the description include each and every individual subcombination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings.

As used herein, “patient” or “subject” refers to a mammal, such as a human. “Patient” and “subject” are used interchangeably herein.

As used herein, “modified nucleotide” refers to a nucleotide that has one or more modifications to the nucleoside, the nucleobase, pentose ring, or phosphate group. For example, modified nucleotides exclude ribonucleotides containing adenosine monophosphate, guanosine monophosphate, uridine monophosphate, and cytidine monophosphate and deoxyribonucleotides containing deoxyadenosine monophosphate, deoxy guano sine monophosphate, deoxythymidine monophosphate, and deoxycytidine monophosphate. Modifications include those naturally occurring that result from modification by enzymes that modify nucleotides, such as methyltransferases. Modified nucleotides also include synthetic or non-naturally occurring nucleotides. Synthetic or non-naturally occurring modifications in nucleotides include those with 2′ modifications, e.g., 2-O-methyl, 2′-methoxyethoxy, 2′-fluoro, 2′-hydroxyl (RNA), 2′-allyl, 2′-O-[2-(methylamino)-2-oxoethyl], 4′-CH2-Oxbridge, 4′-(CH2)2-O-2″-bridge, and 2′-O—(N-methylcarbamate) or those comprising base analogs.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) Proc. Natl. Acad. Sci. USA 87:2264-2268: Altschul, (1993) J. Mol. Evol. 36:290-300; Altschul et al., (1997) Nucleic Acids Res. 25:3389-3402, incorporated by reference herein) are tailored for sequence similarity searching. For a discussion of basic issues in searching sequence databases see Altschul et al., (1994) Nature Genetics 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) Proc. Natl. Acad. Sci. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q=9; R=2; wink=1; and gapw=32. Searches may also be conducted using the NCBI (National Center for Biotechnology Information) BLAST Advanced Option parameter (e.g.; −G, Cost to open gap [Integer]; default=5 for nucleotides/11 for proteins; −E, Cost to extend gap [Integer]; default=2 for nucleotides/1 for proteins; −q, Penalty for nucleotide mismatch [Integer]; default=−3; −r, reward for nucleotide match [Integer]; default=1; −e, expect value [Real]; default=10; −W, wordsize [Integer]; default=11 for nucleotides/28 for megablast/3 for proteins; −y, Dropoff (X) for blast extensions in bits; default=20 for blastn/7 for others; −X, X dropoff value for gapped alignment (in bits); default=15 for all programs, not applicable to blastn; and −Z, final X dropoff value for gapped alignment (in bits); 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

### II. Methods

### A. High Resolution Melting

High Resolution Melting (HRM) is a screening technique which allows the user to determine if the PCR fragment contains a variant or not based upon the dissociation of double-stranded DNA in the presence of an intercalating dye as the temperature increases. As the temperature increases, changes in the fluorescence due to the amplicon dissociation are monitored real-time. It is expected that fragments containing a variant will have slightly different fluorescence profiles than that of wild-type DNA which allows for the entire amplicon to be screened for mutations. Methods for performing HRM are well known in the art (see, for example, Farrar et al. (2017) “High-Resolution Melting Curve Analysis for Molecular Diagnostics,” in Molecular Diagnostics (Third Edition), pp. 79-102:

Since this is a scanning technique and does not identify an exact mutation, any variant present in the PCR product may differentiate from the wild-type DNA, thus leading to a mutation positive result. Positive HRM results may be confirmed by another method, e.g., by sequencing the mutation.

Before an HRM analysis can be performed, the target sequence must be available in high copy number. This may be accomplished by performing a DNA amplification reaction in the presence of a HRM dye before the High Resolution Melt. Exemplary amplification reactions include polymerase chain reaction (PCR), loop mediated isothermal amplification, nucleic acid sequence based amplification, strand displacement amplification, multiple displacement amplification, etc. (See, e.g., Fakruddin et al. (2013) J Pharm Bioallied Sci. 5 (4): 245-252.) Exemplary polymerases include Taq DNA polymerase (e.g., Hot Start Taq DNA polymerase) and AmpliTaq Gold™ 360 DNA Polymerase (Thermo Fisher, Waltham, MA). Exemplary dyes include, for example, LCGreen® (LCGreen® Plus+, LCGreen® Idaho Technology Inc., Salt Lake City, UT), SYTO™ dyes (SYTO™ 9, SYTO™ 11, SYTO™ 12, SYTO™ 13, SYTO™ 14, SYTO™ 15, SYTO™ 16, SYTO™ 18, SYTO™ 20, SYTO™ 21, SYTO™ 22, SYTO™ 23, SYTO™ 24, SYTO™ 25, SYTO™ BC, Molecular Probes, Eugene, OR), EvaGreen® (Biotium, Fremont, CA), SYBR® Green (N,N-dimethyl-N-[4-[(E)-(3-methyl-1,3-benzothiazol-2-ylidene)methyl]-1-phenylquinolin-1-ium-2-yl]-N-propylpropane-1,3-diamine: Molecular Probes, Eugene, OR), and Chai Green™ and those disclosed dyes in WO 2008/052742.

After the amplification reaction, the successive melting experiment can be performed on the same instrument (e.g., a real time instrument), and analyzed with a respective software to identify sequence variants. Any HRM-enabled machine, such as a real time instrument, can be used according to the methods described herein. Exemplary instruments useful in the practice of the methods herein include, for example, 7500 Fast Real-Time PCT System (Thermo Fisher, Waltham, MA), 7900HT Fast Real-Time PCT System (Thermo Fisher, Waltham, MA), and MyGo Pro (Azura Genomics, Raynham, MA). As such, the entire experiment can be done without opening the reaction vessels and without additional handling steps after the PCR setup. Both procedures can be performed in the presence of a fluorescent dye that binds only dsDNA. The dye does not interact with ssDNA, but fluoresces strongly in the presence of dsDNA. This change in fluorescence can be used both to measure the increase in DNA concentration during PCR and then to directly measure thermally-induced DNA dissociation during HRM. In certain embodiments, sample amplification and HRM may be performed in separate vessels and/or using separate instruments.

For detection of sequence variations, differences in the melting curves of the amplicons are analyzed. Heterozygote DNA forms heteroduplexes that begin to separate into single strands at a lower temperature and with a different curve shape than homozygote DNA. Depending on the individual sequence, the different homozygotes may give distinguishable melting curves, too.

In a melting experiment, fluorescence is initially high because the sample starts as dsDNA, but fluorescence diminishes as the temperature is raised and DNA dissociates into single strands. The observed “melting” behavior is characteristic of a particular DNA sample. Mutations in PCR products are detectable because they change the shape of the melting curve. When the mutant sample is compared to a control (e.g., wild-type) sample, these changes are visible.

Usually, an analysis of HRM data is supported by a respective software, which, in some embodiments, provides for the analysis steps of normalization, temperature shifting, a generating a difference plot. Any HRM software can be used according to the methods described herein, including, for example, 7500 Software v2.3 (Thermo Fisher Scientific, Waltham MA) and High Resolution Melt Software v3.1 (Thermo Fisher Scientific, Waltham MA).

Raw melting curve data can be normalized by setting the pre-melt (initial fluorescence) and post-melt (final fluorescence) signals of all samples to uniform values. Pre-melt signals are uniformly set to a relative value of 100%, while post-melt signals are set to a relative value of 0%. Normalizing the initial and final fluorescence in all samples aids interpretation and analysis of the data. In some cases, samples with homozygous SNPs may be distinguished from the wild type by the displacement of their melting curves, which may be easier to see in the normalized data. Software can also be used to generate a temperature-shifted curve, i.e., a normalized melting curve showing a shift on the temperature axis of the at the point where the entire double-stranded DNA is completely denatured. Further information on the differences in melting curve shapes, different can be obtained by means of subtracting the curves from a reference curve (also called “base curve”), thus generating a difference plot, which helps cluster samples automatically into groups that have similar melting curves.

In certain embodiments, samples which test positive for a mutation can be further analyzed by sequencing. Various sequencing methods are known in the art, such as Sanger Sequencing (Yunxia et al. (2010) BMC Med. Genet. 11:34), real-time PCR (Amicarelli et al. (2007) Nucleic. Acids Res. 35: e131) and pyrosequencing (Ogino et al. (2005) J. Mol. Diagn. 7:413-421).

In certain embodiments, at least one of the pairs of primers comprises a tag. In certain embodiments, the tag comprises a sequencing primer such as an M13 primer to facilitate sequencing of the mutation. An M13 primer can include, for example, a M13 forward sequence or an M13 reverse sequence (e.g., AGGAAACAGCTATGACCAT (SEQ ID NO: 187) or TGTAAAACGACGGCCAGT (SEQ ID NO: 188)), or a complement or reverse complement thereof. In certain embodiments, the tag comprises a basic CG clamp sequence (e.g., GCGTCCCG (SEQ ID NO: 189): GCCCCCGCCG (SEQ ID NO: 190): or GCGGCCCGCCGCCCCCGCCG (SEQ ID NO: 191)).

Any sample containing DNA, for example, DNA extracted from whole blood or bone marrow samples, is suitable for use with the methods herein. Other samples suitable for use with the methods herein include tissue, body fluid and/or cell samples.

A method for performing a high resolution melting analysis (HRM) to detect the presence or absence of a mutation in a nucleic acid can include the steps of (1) combining the nucleic acid, at least one primer (e.g., at least one primer pair), a dye, and a reaction mixture comprising one or more buffers, one or more salts, dNTPs, optionally glycerol, a DNA polymerase and optionally a DNA polymerase cofactor: (2) amplifying the nucleic acid; and (3) detecting the presence or absence of the mutation by performing a melting curve analysis of the amplified product.

In certain embodiments, prior to the amplification reaction, nucleic acid is extracted from a sample and resuspended in TE buffer. In certain embodiments, the nucleic acid is at a concentration of at least about 10 ng/μL. In certain embodiments, the nucleic acid is at a concentration of about 10 ng/μL.

In certain embodiments, the reaction mixture comprises the buffer in concentration of from about 5 mM to about 15 mM (e.g., about 10 mM). In certain embodiments, the one or more salts is present in an amount of about 50 mM salt. In certain embodiments, a DNA polymerase cofactor (e.g., MgCl₂) is present, for example, in an amount of about 1.5 mM. In certain embodiments, glycerol is present in an amount between about 5% and about 25% (e.g., 5% or 6%.). In certain embodiments, dNTPs are present in an amount of about 0.2 mM

For example, a method for performing a high resolution melting analysis (HRM) to detect the presence or absence of a mutation in a nucleic acid can include the steps of (1) combining the nucleic acid, at least one primer (e.g., at least one primer pair), a dye, and a reaction mixture comprising 10 mM Tris-HCl, 50 mM KCl, 1.5 mM MgCl₂, 0.2 mM dNTPs, 5% glycerol, and 5-50 units/mL (e.g., 25 units/mL) of a DNA polymerase: (2) amplifying the nucleic acid; and (3) detecting the presence or absence of the mutation by performing a melting curve analysis of the amplified product.

In another example, a method for performing a high resolution melting analysis (HRM) to detect the presence or absence of a mutation in a nucleic acid includes the steps of (1) combining the nucleic acid, at least one primer (e.g., at least one primer pair) and a reaction mixture comprising a DNA polymerase, dUTPs, and a dye: (2) amplifying the nucleic acid; and (3) detecting the presence or absence of the mutation by performing a melting curve analysis of the amplified product.

In certain embodiments, the DNA polymerase comprises a DNA polymerase as described herein. In one embodiment, the DNA polymerase comprises a Hot Start Taq DNA polymerase. In another embodiment, the DNA polymerase comprises a Gold™ 360 DNA Polymerase.

In certain embodiments, the dye is selected from LCGreen® (LCGreen® Plus+, LCGreen® Idaho Technology Inc., Salt Lake City, UT), SYTO™ dyes (SYTO™ 9, SYTO™ 11, SYTO™ 12, SYTO™ 13, SYTO™ 14, SYTO™ 15, SYTO™ 16, SYTO™ 18, SYTO™ 20, SYTO™ 21, SYTO™ 22, SYTO™ 23, SYTO™ 24, SYTO™ 25, SYTO™ BC, Molecular Probes, Eugene, OR), EvaGreen® (Biotium, Fremont, CA), SYBR® Green (N′,N′-dimethyl-N-[4-[(E)-(3-methyl-1,3-benzothiazol-2-ylidene)methyl]-1-phenylquinolin-1-ium-2-yl]-N-propylpropane-1,3-diamine: Molecular Probes, Eugene, OR), Chai Green™ and those dyes disclosed in WO 2008/052742. In one embodiment, the dye comprises MeltDoctor™ HRM Dye. In another embodiment, the dye comprises Chai Green™.

### III. Genes and Primers

In various embodiments of any aspect delineated herein, a primer (e.g., forward and/or reverse primer) comprises one or more 3′ modified nucleotides (e.g., 2′-O-methyl ribonucleotides) in the 3′ terminal region of the primer. In particular embodiments, the primer comprises one or more 2′-O-methyl modified nucleotides at the 3′ end, including for example 2′-O-methyl, 2′-methoxyethoxy, 2′-fluoro, 2′-hydroxyl, 2′-alkyl, 2′-allyl, 2′-O-[2-(methylamino)-2-oxoethyl], 4′-CH2-Oxbridge, 4′-(CH2)2-O-2′-bridge, 2′-LNA, and 2′-O-(Nmethylcarbamate).

In some embodiments, a primer comprises one or more (e.g., two, three, four, five, six, seven, eight, nine, or ten) methylated 3′ nucleotides. For example, a primer for the NOTCH1 gene can comprise the sequence GATCTGGGACTGCATGCTGGTG (SEQ ID NO: 231) or GATCTGGGACTGCATGCTG*G*T*G (SEQ ID NO: 242), wherein (*) denotes methylated 3′ nucleotides.

Without being bound to theory, it is hypothesized that incorporating one or more 3′ modified nucleotides reduces or eliminates intermolecular and/or intramolecular interactions of primers (e.g., primer-dimer formation), and, thereby, reduces or eliminates the background signal in the amplification reaction. The 3′ modified nucleotide preferably has a base that base pairs with the target sequence. In particular embodiments, two or more 3′ modified nucleotides (e.g., 2, 3, 4, 5 or more 3′ modified nucleotides) are used at the 3′ end of the primer (e.g., a block of modified nucleotides). In some embodiments, the block of 3′ modified nucleotides is positioned at the 3′ end of the primer.

### Myeloproliferative Neoplasms (MPN)

### A. MPL

The MPL gene encodes the thrombopoietin receptor protein, which promotes the growth and proliferation of cells, and plays a role in the proliferation of megakaryocytes, which produce platelets. Certain mutations in exon 10 of the MPL gene lead to amino acid changes at W515 of the corresponding protein. These mutations are associated with the development of essential thrombocythemia and/or primary myelofibrosis. Accordingly, the methods described herein are useful in detecting changes in exon 10 of the MPL gene, thereby identifying subjects who have developed or are at risk for developing essential thrombocythemia and/or primary myelofibrosis.

A G to T substitution at nucleotide 1544 of MPL (leading to a W to L amino acid substitution at amino acid 515) and a TG to AA substitution at nucleotides 1543 and 1544 (leading to a W to L amino acid substitution at amino acid 515) are detectable using the methods disclosed herein. In particular, primers comprising SEQ ID NOs: 1 and 2 (see TABLE 1; or primers having the reverse complement thereof or primers having at least 80% or more sequence identity thereto) can be used to detect such changes.

TABLE 1
MPL Primers for Detection of Exon 10 1544G > T or
W515L 1543_1544TG > AA; W515K

| SEQ ID NO: | Sequence |
|---|---|
| 1 | TAGCCTGGATCTCCTTGGTG |
| 2 | GCGGTACCTGTAGTGTGCAG |

### B. CALR

The CALR gene encodes the calreticulin protein, which plays a role in protein folding and calcium regulation, among other things. Certain mutations in exon 9 of the CALR gene lead to a frameshift and truncation of the corresponding protein. These mutations are associated with the development of essential thrombocythemia and/or primary myelofibrosis. Accordingly, the methods described herein are useful in detecting changes in exon 9 of the CALR gene, thereby identifying subjects who have developed or are at risk for developing essential thrombocythemia and/or primary myelofibrosis.

A deletion in exon 9 of CALR (resulting in a frameshift and premature termination (L367fs*46) of the corresponding protein) is detectable using the methods disclosed herein. In addition, a 5-bp insertion in exon 9 of CALR (resulting in a frameshift (K385fs*47) in exon 9, leading to an altered protein having an altered subcellular localization signal) is detectable using the methods disclosed herein. In particular, primers comprising SEQ ID NOs: 3 and 4 (See TABLE 2; or primers having the reverse complement thereof or primers having at least 80% or more sequence identity thereto) can be used to detect such changes.

TABLE 2
CALR Primers for Detection of Exon 9 1092_
1143del, p.L367fs*46 1154_1155insTTGCC;
p.K385fs*47, or 1099_1150del; p.L367fs*46

| SEQ ID NO: | Sequence |
|---|---|
| 3 | CAGAGAAACAAATGAAGGACAAAC |
| 4 | CTTCCTCCTTGTCCTCCTCA |

### C. JAK2

Janus Kinase 2 (JAK2) is a non-receptor tyrosine kinase which lacks Src homology binding domains (SH2/SH3) and contains JAK homology domains. Mutations in exons 12 and 14 of JAK2 are associated with a number of myelodysplastic syndromes (MDS) and myeloproliferative neoplasms (MPN), including leukemia, polycythemia vera, essential thrombocythemia, myelofibrosis. Mutations in exon 13 of JAK2 are associated with a number of myelodysplastic syndromes (MDS) and myeloproliferative neoplasms (MPN), including leukemia, polycythemia vera, essential thrombocythemia, myelofibrosis. Accordingly, the methods described herein are useful in detecting changes in exons 12 and 14 of the JAK2 gene, thereby identifying subjects who have developed or are at risk for developing leukemia, polycythemia vera, essential thrombocythemia, myelofibrosis.

A G to T substitution in exon 14 of JAK2 (resulting in a V to F substitution in amino acid 617 of the corresponding protein) is detectable using the methods disclosed herein. Two deletions and one deletion/insertion in exon 12 of JAK2 are detectable using the methods disclosed herein. One deletion lacks nucleotides 1611 to 1616 (TCACAA) resulting in the deletion of F537 to K539 and insertion of L in the corresponding protein. Another deletion lacks nucleotides 1624 to 1629 (AATGAA) resulting in the deletion of N542 and E543 in the resulting protein. A deletion/insertion deletes nucleotide 1615 and 1616 (AA) and inserts TT, leading to a K to L substitution at amino acid 539 of the resulting protein. A G to A substitution in exon 13 of JAK2 (resulting in a G to S substitution in amino acid 571 of the corresponding protein) is detectable using the methods disclosed herein. Primers comprising SEQ ID NOs: 5, 6, 7, 8, 21, and 22 (See TABLE 3; or primers having the reverse complement thereof or primers having at least 80% or more sequence identity thereto) can be used to detect such changes.

TABLE 3
JAK2 Primers for detecting Exon 14 1849G > T;
p.V617F; Exon 12 1611_1616delTCACAA; p.F537_
K539delinsL, 1624_1629delAATGAA; p.N542_E543del,
or 1615_1616delAAinsTT; p.K539L; or Exon 13
c.1711G > A; p.G571S

| SEQ ID NO: | Sequence | Exon |
|---|---|---|
| 5 | TTTCTTTGAAGCAGCAAGTATG | 14 |
| 6 | AGATGCTCTGAGAAAGGCATTA | 14 |
| 7 | CCAACCTCACCAACATTACAGAG | 12 |
| 8 | TCCAATGTCACATGAATGTAAATCAA | 12 |
| 21 | CTCTTGAAGAATGAAAGCCTTGG | 13 |
| 22 | GTTTCTGTGTGCTTTATCCAGAACT | 13 |

Accordingly, in certain embodiments, the disclosure relates to specific primers which are capable of detecting the mutations disclosed herein. Such primers include oligonucleotides comprising a sequence according to SEQ ID NOs: 1, 2, 3, 4, 5, 6, 7, 8, 21, 22 or their respective complements or oligonucleotides having at least 80%, at least 85%, at least 90%, at least 95%, at least 97%, at least 98%, at least 99% sequence identity to SEQ ID NOs: 1, 2, 3, 4, 5, 6, 7, 8, 21, or 22.

### Acute Myeloid Leukemia (AML)

### A. FLT3

The FLT3 gene encodes the fms-like tyrosine kinase 3, is involved in the normal development of hematopoietic stem cells and progenitor cells. Internal tandem duplications in exons 14, 15, or 16 of FLT3 and mutations in codons 835 and 836 of exon 20 of FLT3 are associated with acute myeloid leukemia (AML). Accordingly, the methods described herein are useful in detecting changes in exons 14, 15, 16, and 20 of the FLT3 gene, thereby identifying subjects who have developed or are at risk for acute myeloid leukemia (AML).

Primers of the disclosure are capable of detecting internal tandem duplications in exons 14, 15, or 16 of FLT3 or mutations in codons 835 and 836 of exon 20 of FLT3. Primers comprising SEQ ID NOs: 26 and 27, SEQ ID NOs: 28 and 29, SEQ ID NOs: 30 and 31, SEQ ID NOs: 32 and 33, or SEQ ID NOs: 234 and 235 (see TABLE 4; or primers having the reverse complement thereof or primers having at least 80% or more sequence identity thereto) can be used to detect such changes.

TABLE 4
FLT3 Primers to Detect Exon 14, 15, and 16
Internal Tandem Duplications or Exon 20
Mutations in Codons 835 And 836

| SEQ ID NO: | Sequence | Exon |
|---|---|---|
| 26 | TGCCTATTCCTAACTGACTCATCATT | 14 |
| 27 | GAAACATTTGGCACATTCCATTC | 14 |
| 28 | AATGCACGTACTCACCATTTGTC | 15 |
| 29 | CATCTTTGTTGCTGTCCTTCCA | 15 |
| 30 | TTACATTTTTAATGCTCCTTTCTTTGA | 16 |
| 31 | GATGAGGTGATTTTCGTGGAAG | 16 |
| 32 | CTTGTCACCCACGGGAAAGT | 20 |
| 33 | AAAATAAGTAGGAAATAGCAGCCTCA | 20 |
| 234 | AGTTTTACATTTTTAATGCTCCTTTCTT | 14 |
| 235 | TGATGAGGTGATTTTCGTGGA | 14 |

### B. IDH2

The IDH2 gene encodes the NADP(+)-dependent isocitrate dehydrogenase protein, found in the mitochondria which plays a role in intermediary metabolism and energy production. Mutations in exon 4 of IDH2 are associated with acute myeloid leukemia (AML). Accordingly, the methods described herein are useful in detecting mutations in exon 4 of the IDH2 gene, thereby identifying subjects who have developed or are at risk for acute myeloid leukemia (AML).

A C to G substitution at nucleotide 418 in exon 4 of IDH2 (resulting in a R to G substitution in amino acid 418 of the corresponding protein), a C to T substitution at nucleotide 418 in exon 4 of IDH2 (resulting in a R to W substitution in amino acid 418 of the corresponding protein), a G to A substitution at nucleotide 419 in exon 4 of IDH2 (resulting in a R to Q substitution in amino acid 419 of the corresponding protein), a G to T substitution at nucleotide 419 in exon 4 of IDH2 (resulting in a R to L substitution in amino acid 419 of the corresponding protein), an A to T substitution at nucleotide 514 in exon 4 of IDH2 (resulting in a R to W substitution in amino acid 514 of the corresponding protein), a G to T substitution at nucleotide 515 in exon 4 of IDH2 (resulting in a R to M substitution in amino acid 172 of the corresponding protein), a G to A substitution at nucleotide 515 in exon 4 of IDH2 (resulting in a R to K substitution in amino acid 172 of the corresponding protein), a G to T substitution at nucleotide 516 in exon 4 of IDH2 (resulting in a R to S substitution in amino acid 172 of the corresponding protein), and a G to C substitution at nucleotide 516 in exon 4 of IDH2 (resulting in a R to S substitution in amino acid 172 of the corresponding protein) are detectable using the methods disclosed herein. Primers comprising SEQ ID NOs: 34 and 35, SEQ ID NOs: 36 and 37, SEQ ID NOs: 238 and 239, or SEQ ID NOs: 240 and 241 (See TABLE 5; or primers having the reverse complement thereof or primers having at least 80% or more sequence identity thereto) can be used to detect such changes.

TABLE 5
IDH2 Primers to Detect Exon 4 c.418C > G;
p.R140G, c.418C > T; p.R140W, c.419G > T;
p.R140L, c.419G > A; p.R140Q, c.514A > T;
p.R172W, c.515G > A; p.R172K, c.515G > T;
p.R172M, c.516G > C; p.R172, or c.516G > T;
p.R172S

| SEQ ID NO: | Sequence |
|---|---|
| 34 | TCTCTGTCCTCACAGAGTTCAAGC |
| 35 | CTTGGTCCAGCCAGGGACTA |
| 36 | CAAAAACATCCCACGCCTAGT |
| 36 | TGGGGTGAAGACCATTTTGAA |
| 238 | GCTGCAGTGGGACCACTATTATC |
| 239 | GCTCCCGGAAGACAGTCCCC |
| 240 | CTGGCTGGACCAAGCCCATCA |
| 241 | CTAGGCGAGGAGCTCCAGTCG |

### C. IDH1

The IDH1 gene encodes the NADP(+)-dependent isocitrate dehydrogenase protein, found in the cytoplasm and peroxisomes. The presence of this enzyme in peroxisomes suggests roles in the regeneration of NADPH for intraperoxisomal reductions as well as in peroxisomal reactions that consume 2-oxoglutarate. The cytoplasmic enzyme serves a significant role in cytoplasmic NADPH production, which plays a role in intermediary metabolism and energy production. Mutations in exon 4 of IDH1 are associated with acute myeloid leukemia (AML). Accordingly, the methods described herein are useful in detecting mutations in exon 4 of the IDH1 gene, thereby identifying subjects who have developed or are at risk for acute myeloid leukemia (AML)

A C to T substitution at nucleotide 298 in exon 4 of IDH1 resulting in premature termination (R100*), a G to A substitution at nucleotide 299 of IDH1 (resulting in a R to Q substitution in amino acid 100 of the corresponding protein), a G to C substitution at nucleotide 313 in exon 4 of IDH1 (resulting in a G to R substitution in amino acid 105 of the corresponding protein), a G to T substitution at nucleotide 314 in exon 4 of IDH1 (resulting in a G to V substitution in amino acid 105 of the corresponding protein), a G to A substitution at nucleotide 314 in exon 4 of IDH1 (resulting in a G to D substitution in amino acid 105 of the corresponding protein), a C to T substitution at nucleotide 394 in exon 4 of IDH1 (resulting in a R to C substitution in amino acid 132 of the corresponding protein), a C to G substitution at nucleotide 394 in exon 4 of IDH1 (resulting in a R to G substitution in amino acid 132 of the corresponding protein), a C to A substitution at nucleotide 394 in exon 4 of IDH1 (resulting in a R to S substitution in amino acid 132 of the corresponding protein), a G to A substitution at nucleotide 395 in exon 4 of IDH1 (resulting in a R to H substitution in amino acid 132 of the corresponding protein), a G to T substitution at nucleotide 395 in exon 4 of IDH1 (resulting in a R to L substitution in amino acid 132 of the corresponding protein), and a G to C substitution at nucleotide 395 in exon 4 of IDH1 (resulting in a R to P substitution in amino acid 132 of the corresponding protein) are detectable using the methods disclosed herein. Primers comprising SEQ ID NOs: 38 and 39 or SEQ ID NOs: 236 and 237 (see TABLE 6: or primers having the reverse complement thereof or primers having at least 80% or more sequence identity thereto) can be used to detect such changes.

TABLE 6
IDH1 Primers to Detect Exon 4 c.298C > T; p.R100*,
c.299G > A; p.R100Q, c.313G > C; p.G105R,
c.314G > T; p.G105V, c.314G > A; p.G105D,
c.394C > A; p.R132S, c.394C > T; p.R132C,
c.394C > G; p.R132G, c.395G > A; p.R132H,
c.395G > T; p.R132L, orc.395G > C; p.R132P

| SEQ ID NO: | Sequence |
|---|---|
| 38 | GAGAAGAGGGTTGAGGAGTTCAA |
| 39 | GCAAAATCACATTATTGCCAACAT |
| 236 | GGCACGGTCTTCAGAGAAGC |
| 237 | ACATTATTGCCAACATGACTTACTTGAT |

### D. Kit

The KIT gene encodes the tyrosine-protein kinase KIT protein. Mutations in exons 9, 11, 13, and 17 of KIT are associated with acute myeloid leukemia (AML) Accordingly, the methods described herein are useful in detecting mutations in exon 9, 11, 13, and 17 of the KIT gene, thereby identifying subjects who have developed or are at risk for acute myeloid leukemia (AML).

A duplication of nucleotides at nucleotides 1504 and 1509 in exon 9 of KIT (resulting in a duplication of A in amino acid 502 to Y in amino acid 503 of the corresponding protein) is detectable using the methods disclosed herein. A deletion of nucleotides at nucleotides 1669 to 1674 in exon 11 of KIT (resulting in a deletion of W in amino acid 557 to K in amino acid 558 of the corresponding protein), a deletion of nucleotides at nucleotides 1669 to 1683 in exon 11 of KIT (resulting in a deletion of W in amino acid 557 to K in amino acid 558 of the corresponding protein), a T to G substitution at nucleotide 1669 in exon 11 of KIT (resulting in a W to G substitution in amino acid 557 of the corresponding protein), a T to C substitution at nucleotide 1669 in exon 11 of KIT (resulting in a W to R substitution in amino acid 557 of the corresponding protein), a T to A substitution at nucleotide 1669 in exon 11 of KIT (resulting in a W to R substitution in amino acid 557 of the corresponding protein), a T to C substitution at nucleotide 1676 in exon 11 of KIT (resulting in a V to A substitution in amino acid 559 of the corresponding protein), a T to A substitution at nucleotide 1676 in exon 11 of KIT (resulting in a V to D substitution in amino acid 559 of the corresponding protein), a T to G substitution at nucleotide 1676 in exon 11 of KIT (resulting in a V to G substitution in amino acid 559 of the corresponding protein), a T to A substitution at nucleotide 1679 in exon 11 of KIT (resulting in a V to D substitution in amino acid 560 of the corresponding protein), and a T to C substitution at nucleotide 1727 in exon 11 of KIT (resulting in a L to P substitution in amino acid 576 of the corresponding protein), are detectable using the methods disclosed herein. An A to G substitution at nucleotide 1924 of KIT (resulting in a K to E substitution in amino acid 642 of the corresponding protein), a T to C substitution at nucleotide 1961 of KIT (resulting in a V to A substitution in amino acid 654 of the corresponding protein), a T to A substitution at nucleotide 1965 of KIT (resulting in a N to K substitution in amino acid 655 of the corresponding protein), are detectable using the methods disclosed herein. An A to G substitution at nucleotide 1924 of KIT (resulting in a K to E substitution in amino acid 642 of the corresponding protein), a T to C substitution at nucleotide 1961 of KIT (resulting in a V to A substitution in amino acid 654 of the corresponding protein), and a T to A substitution at nucleotide 1965 of KIT (resulting in a N to K substitution in amino acid 655 of the corresponding protein), are detectable using the methods disclosed herein. Primers comprising SEQ ID NOs: 40 and 41, SEQ ID NOs: 42 and 43, SEQ ID NOs: 44 and 45, SEQ ID NOs: 46 and 47, or SEQ ID NOs: 232 and 233 (see TABLE 7; or primers having the reverse complement thereof or primers having at least 80% or more sequence identity thereto) can be used to detect such changes.

```
TABLE 7
KIT Primers to Detect Exon 9 1504_1509dup;
p.A502_Y503dup; Exon 11, c.1669_1674del;
  p.W557_K558del, c.1669_1683del; p.W557_E561del,
c.1669T > G; p.W557G, c.1669T > C; p.W557R,
c.1669T > A; p.W557R, c.1676T > C; p.V559A,
c.1676T > A; p.V559D, c.1676T > G; p.V559G,
c.1679T > A; p.V560D, c.1727T > C; p.L576P;
Exon 13 c.1924A > G; p.K642E, c.1961T > C;
p.V654A, or c.1965T > A; p.N655K; or Exon 17
c.2446G > C; p.D816H, c.2466T > A; p.N822K,
c.2446G > T; p.D816Y, c.2446_2447GA > AT;
p.D816I, c.2447A > T; p.D816V, c.2458G > T;
p.D820Y, c.2459A > G; p.D820G, c.2464A > T;
p.N822Y, c.2466T > G; p.N822K, c.2467T > G;
  p.Y823D, or c.2474T > C; p.V825A
```

| SEQ ID NO: | Sequence | Exon |
|---|---|---|
| 40 | CTAGTGCATTCAAGCACAATGG | 9 |
| 41 | CAGAGCCTAAACATCCCCTTAAA | 9 |
| 42 | AGGTGATCTATTTTTCCCTTTCTCC | 11 |
| 43 | TTATGTGTACCCAAAAAGGTGACA | 11 |
| 44 | TGCCAGTTGTGCTTTTTGCT | 13 |
| 45 | ACAATAAAAGGCAGCTTGGACAC | 13 |
| 46 | ACTTGGCAGCCAGAAATATCCTC | 17 |
| 47 | GACTGTCAAGCAGAGAATGGGTACT | 17 |
| 232 | GTATTCACAGAGACTTGGCAGC | 17 |
| 233 | TGTCAAGCAGAGAATGGGTACT | 17 |

### E. NPM1

The NPM1 gene encodes the Nucleophosmin (NPM), also known as nucleolar phosphoprotein B23 or numatrin protein, which has multiple functions, such as histone chaperones; ribosome biogenesis and transport, genomic stability and DNA repair, endoribonuclease activity, centrosome duplication during cell cycle, regulation of ARF-p53 tumor suppressor pathway, RNA helix destabilizing activity, inhibition of caspase-activated DNase, and prevention of apoptosis when located in the nucleolus. Mutations in exon 12 of NPM1 are associated with acute myeloid leukemia (AML). Accordingly, the methods described herein are useful in detecting mutations in exon 12 of the NPM2 gene, thereby identifying subjects who have developed or are at risk for acute myeloid leukemia (AML).

Duplication of nucleotides 860-863 in exon 12 of NPM1 (resulting in a frameshift and premature termination (W288Cfs*12) of the corresponding protein) is detectable using the methods disclosed herein. Primers comprising SEQ ID NOs: 48 and 49 (see TABLE 8; or primers having the reverse complement thereof or primers having at least 80% or more sequence identity thereto) can be used to detect such changes.

TABLE 8
NPM1 Primers to Detect Exon 12 c.860_863dup;
p.W288Cfs*12

| SEQ ID NO: | Sequence |
|---|---|
| 48 | GATGTCTATGAAGTGTTGTGGTTCC |
| 49 | TGTTACAGAAATGAAATAAGACGGAAA |

### F. CEBPA

The CEBPA gene encodes the CCAAT/enhancer-binding protein alpha protein, a transcription factor involved in the differentiation of certain blood cells. Mutations in exon 1 of CEBPA are associated with acute myeloid leukemia (AML). Accordingly, the methods described herein are useful in detecting mutations in exon 1 of the CEBPA gene, thereby identifying subjects who have developed or are at risk for acute myeloid leukemia (AML).

Mutations in exon 1 of CEBPA are detectable using the methods disclosed herein. Primers comprising SEQ ID NOs: 50 and 51, SEQ ID NOs: 52 and 53, SEQ ID NOs: 54 and 55, SEQ ID NOs: 56 and 57, SEQ ID NOs: 58 and 59, SEQ ID NOs: 60 and 61 or SEQ ID NOs: 62 and 63 (see TABLE 9; or primers having the reverse complement thereof or primers having at least 80% or more sequence identity thereto) can be used to detect such changes.

TABLE 9
CEBPA Primers to Detect Exon 1

| SEQ ID NO: | Sequence |
|---|---|
| 50 | GAGGCTCGCCATGCCGGGAG |
| 51 | GATGTCGATGGACGTCTCGTGC |
| 52 | CAGCGCCGCCTTCGGCTTTC |
| 53 | GCCCGGGTAGTCAAAGTCGC |
| 54 | GGCAGCAGGAGAAGGCCAAG |
| 55 | TTCATCCTCCTCGCGGGGCTC |
| 56 | CGGCCGCTGGTGATCAAGC |
| 57 | GGTGACCGGGCTGCAGGTG |
| 58 | CGCACTGCGGCCAGACCAC |
| 59 | GTTGCTGTTCTTGTCCACCGAC |
| 60 | GCGGGCAAGGCCAAGAAGTC |
| 61 | GTGTCCAGTTCGCGGCTCAG |
| 62 | GACCAGTGACAATGACCGCCTG |
| 63 | GGCGACCCCAAACCACTCC |

Accordingly, in certain embodiments, the disclosure relates to specific primers which are capable of detecting the mutations disclosed herein. Such primers include oligonucleotides comprising a sequence according to SEQ ID NOs: 26, 27, 28, 29, 30, 31, 32, 33, 234, 235, 34, 35, 36, 37, 238, 239, 240, 241, 38, 39, 236, 237, 40, 41, 42, 43, 44, 45, 46, 47, 232, 233, 48, 49, 50, 51, 52, 53, 54, 55, 56, 57, 58, 59, 60, 61, 62, 63 or their respective complements or oligonucleotides having at least 80%, at least 85%, at least 90%, at least 95%, at least 97%, at least 98%, at least 99% sequence identity to SEQ ID NOs: 26, 27, 28, 29, 30, 31, 32, 33, 234, 235, 34, 35, 36, 37, 238, 239, 240, 241, 38, 39, 236, 237, 40, 41, 42, 43, 44, 45, 46, 47, 232, 233, 48, 49, 50, 51, 52, 53, 54, 55, 56, 57, 58, 59, 60, 61, 62, 63.

### Anemia (ANM) or Cytopenia

### A. WT1

The WT1 gene encodes the Wilms tumor protein, which has an essential role in the normal development of the urogenital system. Frameshift mutations in exon 8 and mutations in exons 8 and 10 of WT1 are associated with anemia (ANM) and cytopenia. Accordingly, the methods described herein are useful in detecting changes in exons 8 and 10 of the WT1 gene, thereby identifying subjects who have developed or are at risk for anemia (ANM) or cytopenia.

A duplication at nucleotide 1110 in exon 8 of WT1 (resulting in a frameshift (V371Cfs*14) of the corresponding protein), and a C to A substitution at nucleotide 1142 in exon 8 of WT1 (resulting in a premature termination (p.S381*) of the corresponding protein) are detectable using the methods disclosed herein. A C to T substitution at nucleotide 1384 in exon 10 of WT1 (resulting in a R to W substitution in amino acid 462 of the corresponding protein), a G to A substitution at nucleotide 1385 in exon 10 of WT1 (resulting in a R to Q substitution in amino acid 462 of the corresponding protein), a G to C substitution at nucleotide 1385 of Exon 10 of WT1 (resulting in a R to P substitution in amino acid 462 of the corresponding protein), and a G to A substitution at nucleotide 1390 in exon 10 of WT1 (resulting in a D to N substitution in amino acid 464 of the corresponding protein), are detectable using the methods disclosed herein. Primers comprising SEQ ID NOs: 64 and 65, SEQ ID NOs: 66 and 67, SEQ ID NOs: 216 and 217, or SEQ ID NOs: 218 and 219 (See TABLE 10; or primers having the reverse complement thereof or primers having at least 80% or more sequence identity thereto) can be used to detect such changes.

TABLE 10
WT1 Primers to Detect Exon 8 c.1110dup;
p.V371Cfs*14 or c.1142C > A; p.S381*; or Exon 10
c.1384C > T; p.R462W, c.1385G > A; p.R462Q,
c.1385G > C; p.R462P, or c.1390G > A; p.D464N

| SEQ ID NO: | Sequence | Exon |
|---|---|---|
| 64 | CCTTCCTCTTACTCTCTGCCTGC | 8 |
| 65 | GACAGCGGGCACACTTACCAG | 8 |
| 66 | CATTGTTAGGGCCGAGGCTAGA | 10 |
| 67 | ACGCACTTGTTTTACCTGTATGAGTC | 10 |
| 216 | CCACATTGTTAGGGCCGA | 10 |
| 217 | CGCACTTGTTTTACCTGTATGA | 10 |
| 218 | GCCTCCCTTCCTCTTACTCTCT | 8 |
| 219 | GCAGCCTGGGTAAGCACAC | 8 |

### B. ASXL1

The ASXL1 gene encodes the Putative Polycomb group protein, which is necessary for the maintenance of stable repression of homeotic and other loci. The protein is thought to disrupt chromatin in localized areas, enhancing transcription of certain genes while repressing the transcription of other genes. Mutations in exon 12 of ASXL1, including ASXL1 exon 12 codon 591, ASXL1 exon 12 codon 635, ASXL1 exon 12 codon 646, ASXL1 exon 12 codon 693, ASXL1 exon 12 codon 808, and ASXL1 exon 12 codon 1102 are associated with anemia and cytopenia. Accordingly, the methods described herein are useful in detecting changes in exon 12 of the ASXL1 gene, thereby identifying subjects who have developed or are at risk for anemia (ANM) or cytopenia.

A duplication at nucleotide 1772 in exon 12 of ASXL1 (resulting in a premature termination (p.Y591*) of the corresponding protein), deletion of nucleotides at nucleotide 1990 to 1922 in exon 12 of ASXL1 (resulting in a frameshift (p.E635Rfs*15) of the corresponding protein), a duplication at nucleotide 1934 in exon 12 of ASXL1 (resulting in a frameshift (p.G646Wfs*12) of the corresponding protein), a C to T substitution at nucleotide 2077 of ASXL1 exon 12 (resulting in a premature termination (p.R693*) of the corresponding protein), deletion of 1 nucleotide at nucleotide 2423 in exon 12 of ASXL1 (resulting in a frameshift (p.P808Lfs*10) of the corresponding protein), and a G to T substitution at nucleotide 3306 of ASXL1 exon 12 (resulting in a E to D substitution in amino acid 1102 of the corresponding protein) are detectable using the methods disclosed herein. Primers comprising SEQ ID NOs: 68 and 69, SEQ ID NOs: 70 and 71, SEQ ID NOs: 72 and 73, SEQ ID NOs: 74 and 75, SEQ ID NOs: 76 and 77, SEQ ID NOs: 192 and 193, SEQ ID NOs: 194 and 195, SEQ ID NOs: 196 and 197, SEQ ID NOs: 198 and 199, or SEQ ID NOs: 200 and 201 (See TABLE 11; or primers having the reverse complement thereof or primers having at least 80% or more sequence identity thereto) can be used to detect such changes.

TABLE 11
ASXL1 Primers to Detect Exon 12 or Exon 12 codon
591: c.1772dup; p.Y591*, codon 635: c.1900_
1922del; p.E635Rfs*15, codon 646: c.1934dup;
p.G646Wfs*12, codon 693: c.2077C > T; p.R693*,
codon 808: c.2423del; p.P808Lfs*10, or
codon 1102: c.3306G > T; p.E1102D

| SEQ ID NO: | Sequence |
|---|---|
| 68 | GCAGATTCAACTTTCACGTATCAAACC |
| 69 | TGCAGAGCACGGGCTTTAATGT |
| 70 | CCTCGCAGACATTAAAGCCCGT |
| 71 | CCACCATCACCACTGCTGCTG |
| 72 | CAGTGGTGATGGTGGTGAGGC |
| 73 | CCTAGCCCATCTGTGAGTCCAAC |
| 74 | TGTCCTCCCAAACCTCAGTAGC |
| 75 | AGCTTGGCCAGTTCCTTTCTCT |
| 76 | GTGTCTCGAGTATGTGCGGTCC |
| 77 | CATCGTGGGCTGGTGGAAGAA |
| 192 | CTTTCACGTATCAAACCACCC |
| 193 | CACGGGCTTTAATGTCTGC |
| 194 | AAGATCCCAGATTCCCTACTG |
| 195 | GTGGGCTGGTGGAAGAAC |
| 196 | GGCGAGAGGTCACCACTG |
| 197 | CCATCACCACTGCTGCTG |
| 198 | AACTGAATGTGAGTCTGGCA |
| 199 | CACTAGAGACGGAATGGGAC |
| 200 | CAGTGGTGATGGTGGTGAG |
| 201 | CCAACTGTAGCCCTCTGTAG |

### C. RUNX1

The RUNX1 gene encodes the Runt-related transcription factor 1 also known as acute myeloid leukemia 1 protein (AML1) or core-binding factor subunit alpha-2 (CBFA2), which regulates the differentiation of hematopoietic stem cells into mature blood cells. In addition it plays a major role in the development of the neurons that transmit pain. Mutations in exons 4, 5, 6, and 8 of RUNX1 are associated with anemia (ANM) and cytopenia. Accordingly, the methods described herein are useful in detecting changes in exons 4, 5, 6, and 8 of the RUNX1 gene, thereby identifying subjects who have developed or are at risk for anemia (ANM) or cytopenia.

A T to C substitution at nucleotide 167 in exon 4 of RUNX1 (resulting in a L to S substitution in amino acid 56 of the corresponding protein), and a C to T substitution at nucleotide 319 in exon 4 of RUNX1 (resulting in a R to C substitution in amino acid 107 of the corresponding protein) are detectable using the methods disclosed herein. A C to A substitution at nucleotide 422 in exon 5 of RUNX1 (resulting in a premature termination (p.S141*) of the corresponding protein), a G to A substitution at nucleotide 485 in exon 5 of RUNX1 (resulting in a R to K substitution in amino acid 162 of the corresponding protein), and a C to T substitution at nucleotide 496 in exon 5 of RUNX1 (resulting in a premature termination (p.R166*) of the corresponding protein) are detectable using the methods described herein. A G to A substitution at nucleotide 592 in exon 6 of RUNX1 (resulting in a D to N substitution in amino acid 198 of the corresponding protein), a G to A substitution at nucleotide 602 in exon 6 of RUNX1 (resulting in a R to Q substitution in amino acid 201 of the corresponding protein), and a C to T substitution at nucleotide 610 in exon 6 of RUNX1 (resulting in a premature termination (p.R204*) of the corresponding protein) are detectable using the methods described herein. A C to T substitution at nucleotide 958 in exon 8 of RUNX1 (resulting in a premature termination (p.R320*) of the corresponding protein) is detectable using the methods disclosed herein Primers comprising SEQ ID NOs: 78 and 79, SEQ ID NOs: 80 and 81, SEQ ID NOs: 82 and 83, SEQ ID NOs: 84 and 85, SEQ ID NOs: 204 and 205, or SEQ ID NOs: 206 and 207 (see TABLE 12; or primers having the reverse complement thereof or primers having at least 80% or more sequence identity thereto) can be used to detect such changes.

TABLE 12
RUNX1 Primers to Detect Exon 4 c.167T > C;
p.L56S or c.319C > T; p.R107C; Exon 5 c.422C > A;
p.S141*, c.485G > A; p.R162K, or c.496C > T;
p.R166*; Exon 18, c.592G > A; p.D198N,
c.602G > A; p.R201Q, or c.610C > T; p.R204*; or
Exon 8 c.958C > T; p.R320*

| SEQ ID NO: | Sequence | Exon |
|---|---|---|
| 78 | TCCATTGCCTCTCCTTCTGTGC | 4 |
| 79 | TGCCAACTCCTTCATGCACCT | 4 |
| 80 | CCTAGGGGATGTTCCAGATGGC | 5 |
| 81 | AATGTGGGTTTGTTGCCATGAAAC | 5 |
| 82 | TCTATCGTGTCCCCACAGGGAA | 6 |
| 83 | GGGGAAAGGTTGAACCCAAGGA | 6 |
| 84 | CTTCACGCCGCCTTCCACCG | 8 |
| 85 | CTCCGGGCCAGTACCTTGAAA | 8 |
| 204 | AGCAACGCCCATTTCACC | 8 |
| 205 | GCTCAGCTGCAAAGAATGTG | 8 |
| 206 | CAGGCAAGATGAGCGAGGC | 4 |
| 207 | GGGCCAGTACCTTGAAAGCG | 4 |

### D. DNMT3A

The DNMT3A gene encodes DNA (cytosine-5)-methyltransferase 3A, which catalyzes the transfer of methyl groups to specific CpG structures in DNA, a process called DNA methylation. Mutations in exon 23 of DNMT3A are associated with anemia (ANM) and cytopenia. Accordingly, the methods described herein are useful in detecting changes in exon 23 of the DNMT3A gene, thereby identifying subjects who have developed or are at risk for anemia (ANM) or cytopenia.

A C to G substitution at nucleotide 2644 in Exon 23 of DNMT3 (resulting in a R to G substitution in amino acid 882 of the corresponding protein), a C to T substitution at nucleotide 2644 in Exon 23 of DNMT3 (resulting in a R to C substitution in amino acid 882 of the corresponding protein), a C to A substitution at nucleotide 2644 in Exon 23 of DNMT3 (resulting in a R to S substitution in amino acid 882 of the corresponding protein), a G to A substitution at nucleotide 2645 in Exon 23 of DNMT3 (resulting in a R to H substitution in amino acid 882 of the corresponding protein), a G to C substitution at nucleotide 2645 in Exon 23 of DNMT3 (resulting in a R to P substitution in amino acid 882 of the corresponding protein), a G to T substitution at nucleotide 2645 in Exon 23 of DNMT3 (resulting in a R to L substitution in amino acid 882 of the corresponding protein) can lead to an altered protein. Primers comprising SEQ ID NOs: 86 and 87 or SEQ ID NOs: 202 and 203 (See TABLE 13; or primers having the reverse complement thereof or primers having at least 80% or more sequence identity thereto) can be used to detect such changes.

TABLE 13
DNMT3A Primers to Detect Exon 23 c.2644C > G;
p.R882G, c.2644C > T; p.R882C, c.2644C > A;
p.R882S, c.2645G > A; p.R882H, c.2645G > C;
p.R882P, or c.2645G > T; p.R882L

| SEQ ID NO: | Sequence |
|---|---|
| 86 | TCCTGCTGTGTGGTTAGACGG |
| 87 | GAAGAGGTGGCGGATGACTGG |
| 202 | CTGCTGTGTGGTTAGACGG |
| 203 | GAGGTGGCGGATGACTGG |

### E. SF3B1

The SF3B1 gene encodes Splicing factor 3B subunit 1. Splicing factor 3b, together with splicing factor 3a and a 12S RNA unit, forms the U2 small nuclear ribonucleoproteins complex (U2 snRNP). As shown in TABLE 14, mutations in Exon 15, Exon 16, or Exon 17 of SF3B1 are associated with anemia (ANM) and cytopenia. Accordingly, the methods described herein are useful in detecting changes in SF3B1 gene, thereby identifying subjects who have developed or are at risk for anemia (ANM) or cytopenia.

TABLE 14

| | | | |
|---|---|---|---|
| SF3B1 | 15 | c. 1866G > T, G > C | p. E622D |
| | 15 | c. 1873C > T | p. R625C |
| | 15 | c. 1874G > T | p. R625L |
| | 15 | c. 1984C > G | p. H662D |
| | 15 | c. 1986C > G, C > A | p. H662Q |
| | 15 | c. 1996A > C | p. K666Q |
| | 15 | c. 1996A > G | p. K666E |
| | 15 | c. 1997A > C | p. K666T |
| | 15 | c. 1997A > G | p. K666R |
| | 15 | c. 1998G > C, G > T | p. K666N |
| | 16 | c. 2098A > G | p. K700E |
| | 17 | c. 2225G > A | p. G742D |

Mutations in SF3B1 can lead to an altered protein. Primers comprising SEQ ID NOs: 88 and 89, SEQ ID NOs: 90 and 91, SEQ ID NOs: 92 and 93, SEQ ID NOs: 208 and 209, SEQ ID NOs: 210 and 211, SEQ ID NOs: 212 and 213, or SEQ ID NOs: 214 and 215 (See TABLE 15; or primers having the reverse complement thereof or primers having at least 80% or more sequence identity thereto) can be used to detect such changes.

TABLE 15
SF3B1 Primers to Detect Exon 15 c.1866G > T,
G > C p.E622D, c.1873C > T p.R625C, c.1874G > T
p.R625L, c.1984C > G p.H662D, c.1986C > G, C > A
p.H662Q, c.1996A > C p.K666Q, c.1996A > G
p.K666E, c.1997A > C p.K666T, c.1997A > G
p.K666R, c.1998G > C, G > T p.K666N, c.2098A > G
p.K700E, or c.2225G > A p.G742D

| SEQ ID NO: | Sequence |
|---|---|
| 88 | GCCCTGGGCATTCCTTCTTTATT |
| 89 | ACTTCTAAGATGTGGCAAGATGGC |
| 90 | GTTGGGGCATAGTTAAAACCTGTGT |
| 91 | TTCCTCTGTGTTGGCGGATACC |
| 92 | GGTATCCGCCAACACAGAGGAA |
| 93 | AGGAGACTGGAATTCTCGAATAAGGA |
| 208 | GGCTGCTGGTCTGGCTACTAT |
| 209 | CCAGGGCAGAGGCTACAAC |
| 210 | TGTAGGTCTTGTGGATGAGC |
| 211 | TTCCTCTGTGTTGGCGGATA |
| 212 | CAGAGGAAAGGTAAATCCACCA |
| 213 | GGAATAAGATACCCAATAGCCTTCA |
| 214 | TTCCTTCTTTATTGCCCTTCTTA |
| 215 | CAACTTACCATGTTCAATGATTTCAA |

### E. TERT

The TERT gene telomerase reverse transcriptase, which is associated with anemia (ANM) and cytopenia. Accordingly, the methods described herein are useful in detecting changes in the TERT gene, thereby identifying subjects who have developed or are at risk for anemia (ANM) or cytopenia.

Alternatively, in some embodiments, methods described herein do not involve detecting changes in the TERT gene. Thereby, in some embodiments, the methods described herein do not include detecting changes in the TERT gene, and are useful in identifying subjects who have developed or are at risk for anemia (ANM) or cytopenia.

Accordingly, in certain embodiments, the disclosure relates to specific primers which are capable of detecting the mutations disclosed herein. Such primers include oligonucleotides comprising a sequence according to SEQ ID NOs: 64, 65, 66, 67, 216, 217, 218, 219, 68, 69, 70, 71, 72, 73, 74, 75, 76, 77, 192, 193, 194, 195, 196, 197, 198, 199, 200, 201, 78, 79, 80, 81, 82, 83, 84, 85, 204, 205, 206, 207, 86, 87, 202, 203, 88, 89, 90, 91, 92, 93, 208, 209, 210, 211, 212, 213, 214, 215, or their respective complements or oligonucleotides having at least 80%, at least 85%, at least 90%, at least 95%, at least 97%, at least 98%, at least 99% sequence identity to SEQ ID NOs: 64, 65, 66, 67, 216, 217, 218, 219, 68, 69, 70, 71, 72, 73, 74, 75, 76, 77, 192, 193, 194, 195, 196, 197, 198, 199, 200, 201, 78, 79, 80, 81, 82, 83, 84, 85, 204, 205, 206, 207, 86, 87, 202, 203, 88, 89, 90, 91, 92, 93, 208, 209, 210, 211, 212, 213, 214, 215.

### Chronic Lymphocytic Leukemia (CLL)

### A. SF3B1

The SF3B1 gene encodes the splicing factor 3b subunit 1 and is involved in RNA splicing. Mutations in exons 15, 16, or 17 of SF3B1 are associated with chronic lymphocytic leukemia (CLL). Accordingly, the methods described herein are useful in detecting changes in exons 15, 16, or 17 of the SF3B1 gene, thereby identifying subjects who have developed or are at risk for chronic lymphocytic leukemia (CLL).

A G to T substitution at nucleotide 1866 in exon 15 of SF3B1 (resulting in an E to D substitution in amino acid 622 of the corresponding protein), a G to C substitution at nucleotide 1866 in exon 15 of SF3B1 (resulting in an E to D substitution in amino acid 622 of the corresponding protein), a C to T substitution at nucleotide 1873 in exon 15 of SF3B1 (resulting in an R to D substitution in amino acid 625 of the corresponding protein), a G to T substitution at nucleotide 1874 in exon 15 of SF3B1 (resulting in an R to L substitution in amino acid 625 of the corresponding protein), a C to G substitution at nucleotide 1984 in exon 15 of SF3B1 (resulting in an H to D substitution in amino acid 662 of the corresponding protein), a C to G substitution at nucleotide 1986 in exon 15 of SF3B1 (resulting in an H to Q substitution in amino acid 662 of the corresponding protein), a C to A substitution at nucleotide 1986 in exon 15 of SF3B1 (resulting in an H to Q substitution in amino acid 662 of the corresponding protein), an A to C substitution at nucleotide 1996 in exon 15 of SF3B1 (resulting in an K to Q substitution in amino acid 666 of the corresponding protein), an A to G substitution at nucleotide 1996 in exon 15 of SF3B1 (resulting in an K to E substitution in amino acid 666 of the corresponding protein), an A to C substitution at nucleotide 1997 in exon 15 of SF3B1 (resulting in an K to T substitution in amino acid 666 of the corresponding protein), an A to G substitution at nucleotide 1997 in exon 15 of SF3B1 (resulting in an K to R substitution in amino acid 666 of the corresponding protein), a G to T substitution at nucleotide 1998 in exon 15 of SF3B1 (resulting in an K to N substitution in amino acid 666 of the corresponding protein), a G to C substitution at nucleotide 1998 in exon 15 of SF3B1 (resulting in an K to N substitution in amino acid 666 of the corresponding protein), an A to G substitution at nucleotide 2098 in exon 16 of SF3B1 (resulting in an K to E substitution in amino acid 700 of the corresponding protein), or a G to A substitution at nucleotide 2225 in exon 17 of SF3B1 (resulting in an G to D substitution in amino acid 742 of the corresponding protein) are detectable using the methods disclosed herein. An A to G substitution at nucleotide 2098 in exon 16 of SF3B1 (resulting in an K to E substitution in amino acid 700 of the corresponding protein) is detectable using the methods disclosed herein. A G to A substitution at nucleotide 2225 in exon 17 of SF3B1 (resulting in an G to D substitution in amino acid 742 of the corresponding protein) is detectable using the methods disclosed herein. Primers comprising SEQ ID NOs: 88-93 and 208-215 (see TABLE 16; or primers having the reverse complement thereof or primers having at least 80% or more sequence identity thereto) can be used to detect such changes.

TABLE 16
SFB31 Primers to Detect Exon 15 c.1866G > T;
p.E622D, c.1866G > C; p.E622D, c.1873C > T;
p.R625C, c.1874G > T; p.R625L, c.1984C > G;
p.H662D, c.1986C > G; p.H662Q, c.1986C > A;
p.H662Q, c.1996A > C; p.K666Q, c.1996A > G;
p.K666E, c.1997A > C; p.K666T, c.1997A > G;
p.K666R, c.1998G > T; p.K666N, or c.1998G > C;
p.K666N; Exon 16, c.2098A > G; p.K700E; or
Exon 17 c.2225G > A; p.G742D

| SEQ ID NO: | Sequence | Exon |
|---|---|---|
| 88 | GCCCTGGGCATTCCTTCTTTATT | 15 |
| 89 | ACTTCTAAGATGTGGCAAGATGGC | 15 |
| 90 | GTTGGGGCATAGTTAAAACCTGTGT | 16 |
| 91 | TTCCTCTGTGTTGGCGGATACC | 16 |
| 92 | GGTATCCGCCAACACAGAGGAA | 17 |
| 93 | AGGAGACTGGAATTCTCGAATAAGGA | 17 |
| 208 | GGCTGCTGGTCTGGCTACTAT | 15 |
| 209 | CCAGGGCAGAGGCTACAAC | 15 |
| 210 | TGTAGGTCTTGTGGATGAGC | 16 |
| 211 | TTCCTCTGTGTTGGCGGATA | 16 |
| 212 | CAGAGGAAAGGTAAATCCACCA | 17 |
| 213 | GGAATAAGATACCCAATAGCCTTCA | 17 |
| 214 | TTCCTTCTTTATTGCCCTTCTTA | 15 |
| 215 | CAACTTACCATGTTCAATGATTTCAA | 15 |

### B. Notch1

The Notch1 gene encodes a transmembrane receptor involved in the Notch signaling cascade, which plays a role in cell proliferation, stem cell maintenance, and differentiation. Mutations in exon 34 of Notch1 are associated with chronic lymphocytic leukemia (CLL). Accordingly, the methods described herein are useful in detecting mutations in exon 34 of the Notch1 gene, thereby identifying subjects who have developed or are at risk for chronic lymphocytic leukemia (CLL).

Deletion at nucleotide 7541 of Exon 34 of Notch1 (results in a p.P2514Rfs*4 frameshift of the corresponding protein). Primers comprising SEQ ID NOs: 230 and 231 (see TABLE 17; or primers having the reverse complement thereof or primers having at least 80% or more sequence identity thereto) can be used to detect such changes.

TABLE 17
Notch1 Primers to Detect Exon 34 c.7541_
7542delCT; p.P2514Rfs*4

| SEQ ID NO: | Sequence |
|---|---|
| 230 | CTACTCCTCGCCTGTGGACAAC |
| 231 | GATCTGGGACTGCATGCTGGTG |

### C. CXCR4

The CXCR4 gene encodes the C-X-C Motif Chemokine Receptor 4 protein activates signaling pathways for proliferation, cell growth and differentiation. CXCR4 overexpression is associated with more aggressive disease. Studies show an increase in migratory potential in those with high levels of CXCR4. Accordingly, the methods described herein are useful in detecting mutations in exon 2 of the CXCR4 gene, thereby identifying subjects who have developed or are at risk for chronic lymphocytic leukemia (CLL).

A C to T substitution at nucleotide 598 in exon 2 of CXCR4 resulting in premature termination (Q200*), a duplication at nucleotide 952 in exon 4 of CXCR4 (resulting in a frameshift (p.T318Nfs*26) of the corresponding protein), a deletion at nucleotide 959 of CXCR4 (results in a p. V320Efs*23 frameshift of the corresponding protein), a duplication at nucleotide 993 in exon 4 of CXCR4 (resulting in a frameshift (p.G332Rfs*12) of the corresponding protein), an A to T substitution at nucleotide 997 in exon 2 of CXCR4 resulting in premature termination (p.K333*), a C to T substitution at nucleotide 1000 in exon 2 of CXCR4 resulting in premature termination (p.R334*), a duplication at nucleotide 1005 in exon 4 of CXCR4 (resulting in a frameshift (p.G336Wfs*8) of the corresponding protein), a deletion at nucleotide 1012 of CXCR4 (results in a p.S338Lfs*27 frameshift of the corresponding protein), a duplication at nucleotide 1012 in exon 4 of CXCR4 (resulting in a frameshift (p.S338Ffs*6) of the corresponding protein), a C to A substitution at nucleotide 1013 in exon 2 of CXCR4 resulting in premature termination (p.S338*), a C to G substitution at nucleotide 1013 in exon 2 of CXCR4 resulting in premature termination (p.S338*), a deletion at nucleotide 1014 of CXCR4 (results in a p.S339Ffs*26 frameshift of the corresponding protein), and a deletion at nucleotide 1021 of CXCR4 (results in a p.S341Pfs*25 frameshift of the corresponding protein). Primers comprising SEQ ID NOs: 220-223 (see TABLE 18; or primers having the reverse complement thereof or primers having at least 80% or more sequence identity thereto) can be used to detect such changes.

TABLE 18
CXCR3 Primers for Detection of Exon 2 c.598C > T;
p.Q200*, c.952dup; p.T318Nfs*26, c.959_960del;
p.V320Efs*23 c.993dup; p.G332Rfs*12, c.997A > T;
p.K333*, c.1000C > T; p.R334*, c.1005dup;
p.G336Wfs*8, c.1012_1015del; p.S338Lfs*27
c.1012dup; p.S338Ffs*6, c.1013C > A; p.S338*,
c.1013C > G; p.S338, c.1014_1017del;
p.S339Ffs*26, or c.1021 del; p.S341Pfs*25

| SEQ ID NO: | Sequence |
|---|---|
| 220 | CCAACGTCAGTGAGGCAGAT |
| 221 | GGATGACAATACCAGGCAGGA |
| 222 | TGCTTTCCTTGGAGCCAAAT |
| 223 | TGTGTTAGCTGGAGTGAAAACTTG |

### D. MYD88

The MYD88 gene encodes the myeloid differentiation primary response 88 protein involved in signaling within immune cells. Mutations in exons 3, 4, and 5 of MYD88 are associated with chronic lymphocytic leukemia (CLL). Accordingly, the methods described herein are useful in detecting mutations in exon 3, 4, and 5 of the MYD88 gene, thereby identifying subjects who have developed or are at risk for chronic lymphocytic leukemia (CLL).

A G to T substitution at nucleotide 649 in exon 3 of MYD88 (resulting in an V to F substitution in amino acid 217 of the corresponding protein), and a C to G substitution at nucleotide 656 in exon 3 of MYD88 (resulting in an S to C substitution in amino acid 219 of the corresponding protein) are detectable using the methods disclosed herein. A T to C substitution at nucleotide 695 in exon 4 of MYD88 (resulting in a M to T substitution in amino acid 232 of the corresponding protein) and a G to A substitution at nucleotide 728 in exon 4 of MYD88 (resulting in a S to N substitution in amino acid 243 of the corresponding protein) are detectable using the methods disclosed herein. A T to C substitution at nucleotide 794 in exon 5 of MYD88 (resulting in a L to P substitution in amino acid 265 of the corresponding protein) is detectable using the methods disclosed herein. Primers comprising SEQ ID NOs: 224-229 (see TABLE 19; or primers having the reverse complement thereof or primers having at least 80% or more sequence identity thereto) can be used to detect such changes.

TABLE 19
MYD88 Primers for Detection of Exon 3 c.649G > T;
p.V217F or c.656C > G; p.S219C; Exon 4
c.695T > C; p.M232T or c.728G > A; p.S243N;
or Exon 5 c.794T > C; p.L265P

| SEQ ID NO: | Sequence | Exon |
|---|---|---|
| 224 | ATATGCCTGAGCGTTTCGATGC | 3 |
| 225 | GTGGCCTTCTAGCCAACCTCT | 3 |
| 226 | CCCAGGGGATATGCTGAACTA | 4 |
| 227 | ACCTGGAGAGAGGCTGAGTG | 4 |
| 228 | TTGGCTTGCAGGTGCCCATC | 5 |
| 229 | GGCGAGTCCAGAACCAAGATTT | 5 |

Accordingly, in certain embodiments, the disclosure relates to specific primers which are capable of detecting the mutations disclosed herein. Such primers include oligonucleotides comprising a sequence according to SEQ ID NOs: 88, 89, 90, 91, 92, 93, 208, 209, 210, 211, 212, 213, 214, 215, 220, 221, 222, 223, 224, 225, 226, 227, 228, 229, 230, 231, or their respective complements or oligonucleotides having at least 80%, at least 85%, at least 90%, at least 95%, at least 97%, at least 98%, at least 99% sequence identity to SEQ ID NOs: 88, 89, 90, 91, 92, 93, 208, 209, 210, 211, 212, 213, 214, 215, 220, 221, 222, 223, 224, 225, 226, 227, 228, 229, 230, 231.

### BCR/ABL1-Induced Leukemogenesis

A. p210, p190, p230, and p203

The BCR/ABL1 fusion gene is the result of a chromosomal translocation event forming the Philadelphia chromosome that fuses the breakpoint cluster region (BCR) gene to the N-terminus of the abelson tyrosine kinase (ABL1) gene. Depending on the translocation breakpoint in the BCR gene, different Bcr-Abl protein isoforms exist, including p210, p190, p230, and p203.

The p210 (b2a2 or b3a2) isoform (M-BCR), classified as the major breakpoint transcript, is located between exons 13 and 14 in BCR. The presence of the p210 fusion is a molecular marker of chronic myelogenous leukemia (CML) in the stable phase and is associated with durable remission when treated with TKIs. The p210 isoform is found in 25% of B-ALL patients and 3% of AML patients.

The p190 isoform (m-BCR), classified as the minor breakpoint transcript, occurs within BCR exon 1 and is associated with increased monocytosis and absence of basophilia. Isoform p190 exhibits stronger kinase activity compared to isoform p210 and has a tendency to progress to the lymphoid blast phase. The fusion isoform is present in approximately 75% of adult B-ALL, where relapse and TKI resistance is frequent, and overall survival is low.

Isoform p230 is located between BCR exons 19 and 20 and produces a fusion protein, u-BCR, which contains additional BCR coding sequences that are not found in the p190 or p210 variants. Isoform p230 is associated with increased neutrophilic maturation or thrombocytosis and a milder course of CML.

Isoform p203 (b3a3/e14a3 and b2a3/e13a3) is formed with the fusion of BCR breakpoint within exons 13 or 14 with ABL exon 3 instead of the more common exon 2. Isoform p203 is rarely seen in CML. However, rare BCR/ABL1 fusion transcripts may be underreported due to lack of available testing or detection of typical fusion transcripts. Accordingly, the methods described herein are useful in detecting exons 1, 13, or 19 of the BCR gene as well as exon 3 of the ABL gene, thereby identifying subjects who have developed or are at risk for BCR/ABL1-induced leukemogenesis (e.g., chronic myeloid leukemia (CML) or acute lymphocytic leukemia (ALL)).

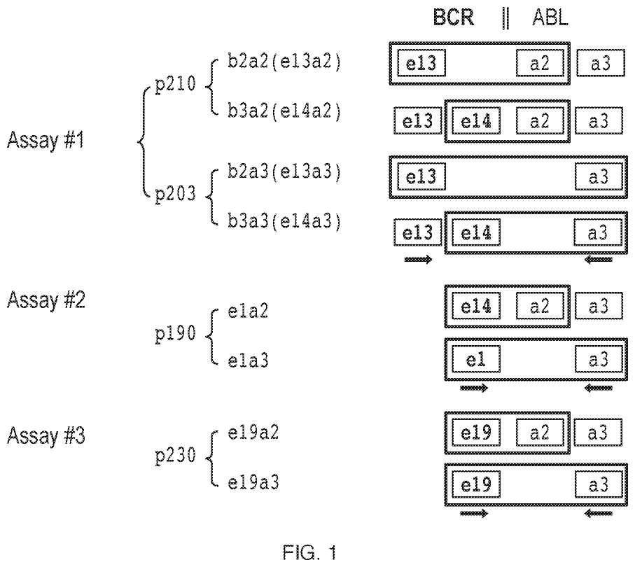

The presence of BCR/ABL1 fusion variants, including four variants of isoforms p210 and p203 between BCR exons 13 and 14 (b2 and b3) and ABL exons 2 and 3 (a2 and a3), two variants of isoform p190 between BCR exons 1 and ABL exons 1 and 2 (e1 and a2) and exon 3 (a3), and two variants of isoform p230 between BCR exon 19 (e19) and ABL exons 2 and 3 (a2 and a3) (FIG. 1) are detectable using the methods disclosed herein. A primer comprising SEQ ID NO: 243-247 (see TABLE 20 and TABLE 21; or primers having the reverse complement thereof or primers having at least 80% or more sequence identity thereto) can be used to detect such changes.

TABLE 20
BCR Primers that Bind to Exons 1, 13, and 19,
Respectively

| SEQ ID NO: | Sequence | Exon |
|---|---|---|
| 243 | ATGCTGACCAACTCGTGTGTGAA | 13 |
| 244 | CAGAACTCGCAACAGTCCTTCGA | 1 |
| 245 | GTGCGTGGAGGAGATCGAGCG | 19 |

TABLE 21
ABL1 Primers that Bind to Exon 3

| SEQ ID NO: | Sequence |
|---|---|
| 246 | TGATTATAGCCTAAGACCCGGAGC |
| 247 | CCTAAGACCCGGAGCTTTTCAC |

SEQ ID NO: 243 binds a portion of exon 13 of BCR, SEQ ID NO: 244 binds a portion of exon 1 of BCR, and SEQ ID NO: 245 binds a portion of exon 19 of BCR. SEQ ID NOs: 246 and 247 each binds a portion exon 3 of ABL1, SEQ ID NO: 243 can be used with either or both of SEQ ID NO: 246 or SEQ ID NO: 247 to amplify a gene encoding BCR/ABL1 isoform p203 or a gene encoding BCR/ABL1 isoform p 210, SEQ ID NO: 244 can be used with either or both of SEQ ID NO: 246 or SEQ ID NO: 247 to amplify a gene encoding BCR/ABL1 isoform p190. SEQ ID NO: 245 can be used with either or both of SEQ ID NO: 246 or SEQ ID NO: 247 to amplify a gene encoding BCR/ABL isoform p230.

In certain embodiments, a primer that binds to BCR exon 13 (e.g., SEQ ID NO: 243) is used in combination with one or more primers that binds to ABL1 Exon 3 (e.g., SEQ ID NOs: 246 and/or 247). In certain embodiments, a primer that binds to BCR exon 1 (e.g., SEQ ID NO: 244) is used in combination with one or more primers that binds to ABL1 Exon 3 (e.g., SEQ ID NOs: 246 and/or 247). In certain embodiments, a primer that binds to BCR exon 19 (e.g., SEQ ID NO: 245) is used in combination with one or more primers that binds to ABL1 Exon 3 (e.g., SEQ ID NOs: 246 and/or 247).

Accordingly, in certain embodiments, the disclosure relates to specific primers which are capable of detecting the exons disclosed herein. Such primers include oligonucleotides comprising a sequence according to SEQ ID NOs: 243, 244, 245, 246, 247, or their respective complements or oligonucleotides having at least 80%, at least 85%, at least 90%, at least 95%, at least 97%, at least 98%, at least 99% sequence identity to SEQ ID NOs: 243, 244, 245, 246, 247.

### IV. Treatment

### Myeloproliferative Neoplasms (MPN)

The disclosure relates in part to a method of treating a subject in need of treatment of a myeloid malignancy. The method can include amplifying a region of a gene of interest from a sample from the subject to produce a double-stranded oligonucleotide, wherein the amplifying step comprises performing PCR using a pair of primers, selected from the group consisting of:

i)
(SEQ ID NO: 1)
TAGCCTGGATCTCCTTGGTG
and
(SEQ ID NO: 2)
GCGGTACCTGTAGTGTGCAG;
(ii)
(SEQ ID NO: 3)
GCAGAGAAACAAATGAAGGACAAAC
and
(SEQ ID NO: 4)
CTTCCTCCTTGTCCTCCTCA;
(iii)
(SEQ ID NO: 5)
TTTCTTTGAAGCAGCAAGTATG
and
(SEQ ID NO: 6)
AGATGCTCTGAGAAAGGCATTA;
(iv)
(SEQ ID NO: 7)
CCAACCTCACCAACATTACAGAG
and
(SEQ ID NO: 8)
TCCAATGTCACATGAATGTAAATCAA;
(v)
(SEQ ID NO: 21)
CTCTTGAAGAATGAAAGCCTTGG
and
(SEQ ID NO: 22)
GTTTCTGTGTGCTTTATCCAGAACT;

(vi) a pair of primers having the reverse complement sequences of any of (i) through (v); and
(vii) a pair of primers having at least 90% sequence identity to any of (i) through (vi);

The method can also include determining that the double-stranded oligonucleotide comprises or does not comprise the mutation in the gene of interest by means of a High Resolution Melting (HRM) analysis. Where the mutation is present, the method can also include sequencing the mutation. The method can also include treating the subject in need of treatment of the myeloid malignancy with an effective amount of a therapeutic agent, as described in more detail below.

Treatment for polycythemia vera is aimed at reducing the number of extra blood cells. Treatment of polycythemia vera may include, phlebotomy, chemotherapy with or without phlebotomy, or biologic therapy using interferon alfa or pegylated interferon alpha and low-dose aspirin.

The treatment of primary myelofibrosis in patients without signs or symptoms is usually watchful waiting. Patients with primary myelofibrosis may have signs or symptoms of anemia. Anemia is usually treated with transfusion of red blood cells to relieve symptoms and improve quality of life. In addition, anemia may be treated with erythropoietic growth factors, prednisone, danazol, thalidomide, lenalidomide, or pomalidomide. Treatment of primary myelofibrosis in patients with other signs or symptoms may include targeted therapy with ruxolitinib (a JAK1 and JAK2 inhibitor), chemotherapy; donor stem cell transplant; thalidomide; lenalidomide; or pomalidomide; splenectomy; radiation therapy to the spleen, lymph nodes, or other areas outside the bone marrow where blood cells are forming; biologic therapy using interferon alfa or erythropoietic growth factors; or the inclusion in a clinical trial of other targeted therapy drugs.

Treatment of essential thrombocythemia in patients younger than 60 years who have no signs or symptoms and an acceptable platelet count is usually watchful waiting. In some cases, the patient can take aspirin to help prevent blood clots. Treatment of other patients may include chemotherapy, hydroxyurea, anagrelide therapy, biologic therapy using interferon alfa or pegylated interferon alpha, or platelet apheresis.

The JAK-binding inhibitor ruxolitinib ((3R)-3-cyclopentyl-3-[4-(7H-pyrrolo[2,3-d]pyrimidin-4-yl) pyrazol-1-yl]pr-opanenitrile: trade name Jakafi, Jakavi) is approved for use in the treatment of high and intermediate risk myelofibrosis: see Tefferi (2012) Blood: 119 (12) Ruxolitinib is also used in the therapy of myelofibrosis: see Ostojic (2012) Therapeutics and Clinical Risk Management 8:95-103.

Other JAK inhibitors suitable for use herein include SAR302503, CYT387, lestaurtinib, SB1518, AZD1480, BMS911543, LY2784544, NS-018, and XL019: see Tefferi, supra.

Refractory anemia with ringed sideroblast and thrombocytosis may require blood transfusions and other supportive therapy to remedy anemia, including high doses of pyrodoxine (Vitamin B6). Bone marrow transplant is also an option. RARS-T may also progress to leukemia.

The use of above therapies is contemplated for patients diagnosed positive for the presence of mutant MPL, CALR, and/or JAK2 in accordance with the present invention, either alone or in combination with therapies (e.g. antibodies) specifically targeting the mutant forms of the respective protein (if applicable). Accordingly, therapies (e.g. antibodies) that target mutant MPL, CALR, and/or JAK2, can likewise be useful in treatment if used as monotherapy or in combination with other therapies. If, for example, the patient is tested positive for the presence of mutant calreticulin and (a) JAK2 mutation(s), the use of JAK inhibitor(s) (like ruxolitinib) is contemplated herein. Depending on clinical parameters, (e.g., age, prognosis of the patient) also further therapies, like stem cell transplantation can be used to treat e.g. a patient tested positive for the presence of mutant MPL, CALR, and/or JAK2.

### Acute Myeloid Leukemia (AML)

The disclosure relates in part to a method of treating a subject in need of treatment of acute myeloid leukemia (AML). The method can include amplifying a region of a gene of interest from a sample from the subject to produce a double-stranded oligonucleotide, wherein the amplifying step comprises performing PCR using a pair of primers, selected from the group

(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 30)
TTACATTTTTAATGCTCCTTTCTTTGA
and
(SEQ ID NO: 31)
GATGAGGTGATTTTCGTGGAAG;
(iv)
(SEQ ID NO: 32)
CTTGTCACCCACGGGAAAGT
and
(SEQ ID NO: 33)
AAAATAAGTAGGAAATAGCAGCCTCA;
(v)
(SEQ ID NO: 234)
AGTTTTACATTTTTAATGCTCCTTTCTT
and
(SEQ ID NO: 235)
TGATGAGGTGATTTTCGTGGA;
(vi)
(SEQ ID NO: 34)
TCTCTGTCCTCACAGAGTTCAAGC
and
(SEQ ID NO: 35)
CTTGGTCCAGCCAGGGACTA;
(vii)
(SEQ ID NO: 36)
CAAAAACATCCCACGCCTAGT
and
(SEQ ID NO: 37)
TGGGGTGAAGACCATTTTGAA;
(viii)
(SEQ ID NO: 238)
GCTGCAGTGGGACCACTATTATC;
and
(SEQ ID NO: 239)
GCTCCCGGAAGACAGTCCCC;
(ix)
(SEQ ID NO: 240)
CTGGCTGGACCAAGCCCATCA;
and
(SEQ ID NO: 241)
CTAGGCGAGGAGCTCCAGTCG;
(x)
(SEQ ID NO: 38)
GAGAAGAGGGTTGAGGAGTTCAA
and
(SEQ ID NO: 39)
GCAAAATCACATTATTGCCAACAT;
(xi)
(SEQ ID NO: 236)
GGCACGGTCTTCAGAGAAGC;
and
(SEQ ID NO: 237)
ACATTATTGCCAACATGACTTACTTGAT;
(xii)
(SEQ ID NO: 40)
CTAGTGCATTCAAGCACAATGG
and
(SEQ ID NO: 41)
CAGAGCCTAAACATCCCCTTAAA;
(xii)
(SEQ ID NO: 42)
AGGTGATCTATTTTTCCCTTTCTCC
and
(SEQ ID NO: 43)
TTATGTGTACCCAAAAAGGTGACA;
(xiv)
(SEQ ID NO: 44)
TGCCAGTTGTGCTTTTTGCT
and
(SEQ ID NO: 45)
ACAATAAAAGGCAGCTTGGACAC;
(xv)
(SEQ ID NO: 46)
ACTTGGCAGCCAGAAATATCCTC
and
(SEQ ID NO: 47)
GACTGTCAAGCAGAGAATGGGTACT;
(xvi)
(SEQ ID NO: 232)
GTATTCACAGAGACTTGGCAGC;
and
(SEQ ID NO: 233)
TGTCAAGCAGAGAATGGGTACT;
(xvii)
(SEQ ID NO: 48)
GATGTCTATGAAGTGTTGTGGTTCC
and
(SEQ ID NO: 49)
TGTTACAGAAATGAAATAAGACGGAAA;
(xviii)
(SEQ ID NO: 50)
GAGGCTCGCCATGCCGGGAG
and
(SEQ ID NO: 51)
GATGTCGATGGACGTCTCGTGC;
(xix)
(SEQ ID NO: 52)
CAGCGCCGCCTTCGGCTTTC
and
(SEQ ID NO: 53)
GCCCGGGTAGTCAAAGTCGC;
(xx)
(SEQ ID NO: 54)
GGCAGCAGGAGAAGGCCAAG
and
(SEQ ID NO: 55)
TTCATCCTCCTCGCGGGGCTC;
(xxi)
(SEQ ID NO: 56)
CGGCCGCTGGTGATCAAGC
and
(SEQ ID NO: 57)
GGTGACCGGGCTGCAGGTG;
(xxii)
(SEQ ID NO: 58)
CGCACTGCGGCCAGACCAC
and
(SEQ ID NO: 59)
GTTGCTGTTCTTGTCCACCGAC;
(xxiii)
(SEQ ID NO: 60)
GCGGGCAAGGCCAAGAAGTC
and
(SEQ ID NO: 61)
GTGTCCAGTTCGCGGCTCAG;
(xxiv)
(SEQ ID NO: 62)
GACCAGTGACAATGACCGCCTG
and
(SEQ ID NO: 63)
GGCGACCCCAAACCACTCC;

(xxv) a pair of primers having the reverse complement sequences of any of (i) through (xxiv); and
(xxvi) a pair of primers having at least 90% sequence identity to any of (i) through (xxv).

The method can also include determining that the double-stranded oligonucleotide comprises or does not comprise the mutation in the gene of interest by means of a High Resolution Melting (HRM) analysis. Where the mutation is present, the method can also include sequencing the mutation. The method can also include treating the subject in need of treatment of the acute myeloid leukemia (AML) malignancy with an effective amount of a therapeutic agent, as described in more detail below.

The use of above therapies is contemplated for patients diagnosed positive for the presence of mutant FLT3, IDH2, IDH1, KIT NPM1, and/or CEBPA in accordance with the present invention, either alone or in combination with therapies (e.g. antibodies) specifically targeting the mutant forms of the respective protein (if applicable). Accordingly, therapies (e.g. antibodies) that target mutant FLT3, IDH2, IDH1, KIT NPM1, and/or CEBPA, can likewise be useful in treatment if used as monotherapy or in combination with other therapies. Depending on clinical parameters, (e.g., age, prognosis of the patient) also further therapies, like stem cell transplantation can be used to treat e.g. a patient tested positive for the presence of mutant FLT3, IDH2, IDH1, KIT NPM1, and/or CEBPA.

### Anemia (ANM) or Cytopenia

The disclosure relates in part to a method of treating a subject in need of treatment of a anemia (ANM) or cytopenia. The method can include amplifying a region of a gene of interest from a sample from the subject to produce a double-stranded oligonucleotide, wherein the amplifying step comprises performing PCR using a pair of primers, selected from the group

(i)
(SEQ ID NO: 64)
CCTTCCTCTTACTCTCTGCCTGC
and
(SEQ ID NO: 65)
GACAGCGGGCACACTTACCAG;
(ii)
(SEQ ID NO: 66)
CATTGTTAGGGCCGAGGCTAGA
and
(SEQ ID NO: 67)
ACGCACTTGTTTTACCTGTATGAGTC;
(iii)
(SEQ ID NO: 218)
GCCTCCCTTCCTCTTACTCTCT;
and
GCAGCCTGGGTAAGCACAC;
(iv)
(SEQ ID NO: 68)
GCAGATTCAACTTTCACGTATCAAACC
and
(SEQ ID NO: 69)
TGCAGAGCACGGGCTTTAATGT;
(v)
(SEQ ID NO: 70)
CCTCGCAGACATTAAAGCCCGT
and
(SEQ ID NO: 71)
CCACCATCACCACTGCTGCTG;
(vi)
(SEQ ID NO: 72)
CAGTGGTGATGGTGGTGAGGC
and
(SEQ ID NO: 73)
CCTAGCCCATCTGTGAGTCCAAC;
(vii)
(SEQ ID NO: 74)
TGTCCTCCCAAACCTCAGTAGC
and
(SEQ ID NO: 75)
AGCTTGGCCAGTTCCTTTCTCT;
(viii)
(SEQ ID NO: 76)
GTGTCTCGAGTATGTGCGGTCC
and
(SEQ ID NO: 77)
CATCGTGGGCTGGTGGAAGAA;
(ix)
(SEQ ID NO: 192)
CTTTCACGTATCAAACCACCC
and
(SEQ ID NO: 193)
CACGGGCTTTAATGTCTGC;
(x)
(SEQ ID NO: 194)
AAGATCCCAGATTCCCTACTG
and
(SEQ ID NO: 195)
GTGGGCTGGTGGAAGAAC;
(xi)
(SEQ ID NO: 196)
GGCGAGAGGTCACCACTG
and
(SEQ ID NO: 197)
CCATCACCACTGCTGCTG;
(xii)
(SEQ ID NO: 198)
AACTGAATGTGAGTCTGGCA
and
(SEQ ID NO: 199)
CACTAGAGACGGAATGGGAC;
(xiii)
(SEQ ID NO: 200)
CAGTGGTGATGGTGGTGAG
and
(SEQ ID NO: 201)
CCAACTGTAGCCCTCTGTAG;
(xiv)
(SEQ ID NO: 78)
TCCATTGCCTCTCCTTCTGTGC
and
(SEQ ID NO: 79)
TGCCAACTCCTTCATGCACCT;
(xv)
(SEQ ID NO: 80)
CCTAGGGGATGTTCCAGATGGC
and
(SEQ ID NO: 81)
AATGTGGGTTTGTTGCCATGAAAC;
(xvi)
(SEQ ID NO: 82)
TCTATCGTGTCCCCACAGGGAA
and
(SEQ ID NO: 83)
GGGGAAAGGTTGAACCCAAGGA;
(xvii)
(SEQ ID NO: 84)
CTTCACGCCGCCTTCCACCG
and
(SEQ ID NO: 85)
CTCCGGGCCAGTACCTTGAAA;
(xviii)
(SEQ ID NO: 204)
AGCAACGCCCATTTCACC
and
(SEQ ID NO: 205)
GCTCAGCTGCAAAGAATGTG;
(xix)
(SEQ ID NO: 206)
CAGGCAAGATGAGCGAGGC
and
(SEQ ID NO: 207)
GGGCCAGTACCTTGAAAGCG;
(xx)
(SEQ ID NO: 86)
TCCTGCTGTGTGGTTAGACGG
and
(SEQ ID NO: 87)
GAAGAGGTGGCGGATGACTGG;
(xxi)
(SEQ ID NO: 202)
CTGCTGTGTGGTTAGACGG
and
(SEQ ID NO: 203)
GAGGTGGCGGATGACTGG;
(xxii)
(SEQ ID NO: 88)
GCCCTGGGCATTCCTTCTTTATT
and
(SEQ ID NO: 89)
ACTTCTAAGATGTGGCAAGATGGC;
(xxiii)
(SEQ ID NO: 90)
GTTGGGGCATAGTTAAAACCTGTGT
and
(SEQ ID NO: 91)
TTCCTCTGTGTTGGCGGATACC;
(xxiv)
(SEQ ID NO: 92)
GGTATCCGCCAACACAGAGGAA
and
(SEQ ID NO: 93)
AGGAGACTGGAATTCTCGAATAAGGA;
(xxv)
(SEQ ID NO: 208)
GGCTGCTGGTCTGGCTACTAT
and
(SEQ ID NO: 209)
CCAGGGCAGAGGCTACAAC;
(xxvi)
(SEQ ID NO: 210)
TGTAGGTCTTGTGGATGAGC
and
(SEQ ID NO: 211)
TTCCTCTGTGTTGGCGGATA;
(xxvii)
(SEQ ID NO: 212)
CAGAGGAAAGGTAAATCCACCA
and
(SEQ ID NO: 213)
GGAATAAGATACCCAATAGCCTTCA;
(xxviii)
(SEQ ID NO: 214)
TTCCTTCTTTATTGCCCTTCTTA
and
(SEQ ID NO: 215)
CAACTTACCATGTTCAATGATTTCAA;

(xxix) a pair of primers having the reverse complement sequences of any of (i) through (xxviii); and
(xxx) a pair of primers having at least 90% sequence identity to any of (i) through (xxix).

The method can also include determining that the double-stranded oligonucleotide comprises or does not comprise the mutation in the gene of interest by means of a High Resolution Melting (HRM) analysis. Where the mutation is present, the method can also include sequencing the mutation. The method can also include treating the subject in need of treatment of the anemia (ANM) malignancy with an effective amount of a therapeutic agent, as described in more detail below. The method can also include treating the subject in need of treatment of the cytopenia malignancy with an effective amount of a therapeutic agent, as described in more detail below.

The use of above therapies is contemplated for patients diagnosed positive for the presence of mutant WT1, ASXL1, RUNX1, DNMT3, SF3B1, and/or TERT in accordance with the present invention, either alone or in combination with therapies (e.g. antibodies) specifically targeting the mutant forms of the respective protein (if applicable). Accordingly, therapies (e.g. antibodies) that target mutant WT1, ASXL1, RUNX1, DNMT3, SF3B1, and/or TERT can likewise be useful in treatment if used as monotherapy or in combination with other therapies. Depending on clinical parameters, (e.g., age, prognosis of the patient) also further therapies, like stem cell transplantation can be used to treat e.g. a patient tested positive for the presence of mutant WT1, ASXL1, RUNX1, DNMT3, SF3B1, and/or TERT.

### V. Kits

Some illustrative embodiments of kits disclosed herein include at least one pair of amplification primers as disclosed above and herein. Some embodiments of kits disclosed herein may further comprise one, several, or all of the following additional ingredients; which may be suited to be used for HRM.

### Chronic Lymphocytic Leukemia (CLL)

The disclosure relates in part to a method of treating a subject in need of treatment of a chronic lymphocytic leukemia (CLL). The method can include amplifying a region of a gene of interest from a sample from the subject to produce a double-stranded oligonucleotide, wherein the amplifying step comprises performing PCR using a pair of primers, selected from the group

(i)
(SEQ ID NO: 88)
GCCCTGGGCATTCCTTCTTTATT
and
(SEQ ID NO: 89)
ACTTCTAAGATGTGGCAAGATGGC;
(ii)
(SEQ ID NO: 90)
GTTGGGGCATAGTTAAAACCTGTGT
and
(SEQ ID NO: 91)
TTCCTCTGTGTTGGCGGATACC;
(iii)
(SEQ ID NO: 92)
GGTATCCGCCAACACAGAGGAA
and
(SEQ ID NO: 93)
AGGAGACTGGAATTCTCGAATAAGGA;
(iv)
(SEQ ID NO: 208)
GGCTGCTGGTCTGGCTACTAT
and
(SEQ ID NO: 209)
CCAGGGCAGAGGCTACAAC;
(v)
(SEQ ID NO: 210)
TGTAGGTCTTGTGGATGAGC
and
(SEQ ID NO: 211)
TTCCTCTGTGTTGGCGGATA;
(vi)
(SEQ ID NO: 212)
CAGAGGAAAGGTAAATCCACCA
and
(SEQ ID NO: 213)
GGAATAAGATACCCAATAGCCTTCA;
(vii)
(SEQ ID NO: 214)
TTCCTTCTTTATTGCCCTTCTTA
and
(SEQ ID NO: 215)
CAACTTACCATGTTCAATGATTTCAA;
(viii)
(SEQ ID NO: 230)
CTACTCCTCGCCTGTGGACAAC
and
(SEQ ID NO: 231)
GATCTGGGACTGCATGCTGGTG;
(ix)
(SEQ ID NO: 220)
CCAACGTCAGTGAGGCAGAT
and
(SEQ ID NO: 221)
GGATGACAATACCAGGCAGGA;
(x)
(SEQ ID NO: 222)
TGCTTTCCTTGGAGCCAAAT
and
(SEQ ID NO: 223)
TGTGTTAGCTGGAGTGAAAACTTG;
(xi)
(SEQ ID NO: 224)
ATATGCCTGAGCGTTTCGATGC
and
(SEQ ID NO: 225)
GTGGCCTTCTAGCCAACCTCT;
(xii)
(SEQ ID NO: 226)
CCCAGGGGATATGCTGAACTA
and
(SEQ ID NO: 227)
ACCTGGAGAGAGGCTGAGTG;
(xiii)
(SEQ ID NO: 228)
TTGGCTTGCAGGTGCCCATC
and
(SEQ ID NO: 229)
GGCGAGTCCAGAACCAAGATTT;

(xiv) a pair of primers having the reverse complement sequences of any of (i) through (xiii); and
(xv) a pair of primers having at least 90% sequence identity to any of (i) through (xiv).

The method can also include determining that the double-stranded oligonucleotide comprises or does not comprise the mutation in the gene of interest by means of a High Resolution Melting (HRM) analysis. Where the mutation is present, the method can also include sequencing the mutation. The method can also include treating the subject in need of treatment of the chronic lymphocytic leukemia (CLL) malignancy with an effective amount of a therapeutic agent, as described in more detail below.

The use of above therapies is contemplated for patients diagnosed positive for the presence of mutant SF3B1, NOTCH1, CXCR4, and/or MYD88 in accordance with the present invention, either alone or in combination with therapies (e.g. antibodies) specifically targeting the mutant forms of the respective protein (if applicable). Accordingly, therapies (e.g. antibodies) that target mutant SF3B1, NOTCH1, CXCR4, and/or MYD88, can likewise be useful in treatment if used as monotherapy or in combination with other therapies. Depending on clinical parameters, (e.g., age, prognosis of the patient) also further therapies, like stem cell transplantation can be used to treat e.g. a patient tested positive for the presence of mutant SF3B1, NOTCH1, CXCR4, and/or MYD88.

### V. Kits

Some illustrative embodiments of kits disclosed herein include at least one pair of amplification primers as disclosed above and herein. Some embodiments of kits disclosed herein may further comprise one, several, or all of the following additional ingredients: which may be suited to be used for HRM.

### BCR/ABL1-Induced Leukemogenesis

The disclosure relates in part to a method of treating a subject in need of treatment of a BCR/ABL1-induced leukemogenesis (e.g., chronic myeloid leukemia (CML) or acute lymphocytic leukemia (ALL)). The method can include amplifying a region of a gene of interest from a sample from the subject to produce a double-stranded oligonucleotide, wherein the amplifying step comprises performing PCR using a primer, selected from the group consisting of:

(i)
(SEQ ID NO: 243)
ATGCTGACCAACTCGTGTGTGAA;
(ii)
(SEQ ID NO: 244)
CAGAACTCGCAACAGTCCTTCGA;
(iii)
(SEQ ID NO: 245)
GTGCGTGGAGGAGATCGAGCG;
(iv)
(SEQ ID NO: 246)
TGATTATAGCCTAAGACCCGGAGC;
(v)
(SEQ ID NO: 247)
CCTAAGACCCGGAGCTTTTCAC;

(vi) two or more primers having the reverse complement sequences of any of (i) through (v); and
(vii) two or more primers having at least 90% sequence identity to any of (i) through (vi).

The method can also include determining that the double-stranded oligonucleotide comprises or does not comprise the exon in the gene of interest by means of a High Resolution Melting (HRM) analysis. Where the exon is present, the method can also include sequencing the exon. The method can also include treating the subject in need of treatment of the BCR/ABL1-induced leukemogenesis (e.g., chronic myeloid leukemia (CML) or acute lymphocytic leukemia (ALL)) malignancy with an effective amount of a therapeutic agent, as described in more detail below.

The use of above therapies is contemplated for patients diagnosed positive for the presence of a BCR/ABL1 fusion gene in accordance with the present invention, either alone or in combination with therapies (e.g. antibodies) specifically targeting the mutant forms of the respective protein (if applicable). Accordingly, therapies (e.g. antibodies) that target BCR/ABL1 fusion proteins, can likewise be useful in treatment if used as monotherapy or in combination with other therapies. Depending on clinical parameters, (e.g., age, prognosis of the patient) also further therapies, like stem cell transplantation can be used to treat e.g. a patient tested positive for the presence of BCR/ABL1 fusion proteins.

### V. Kits

Some illustrative embodiments of kits disclosed herein include at least one pair of amplification primers as disclosed above and herein. Some embodiments of kits disclosed herein may further comprise one, several, or all of the following additional ingredients; which may be suited to be used for HRM.

### Myeloproliferative Neoplasms (MPN)

In certain embodiments, the kit includes at least one pair of primers capable of amplifying a region of a gene of interest from a sample to produce a double-stranded oligonucleotide. Suitable primer pairs for use herein include:

i)
(SEQ ID NO: 1)
TAGCCTGGATCTCCTTGGTG
and
(SEQ ID NO: 2)
GCGGTACCTGTAGTGTGCAG;
(ii)
(SEQ ID NO: 3)
GCAGAGAAACAAATGAAGGACAAAC
and
(SEQ ID NO: 4)
CTTCCTCCTTGTCCTCCTCA;
(iii)
(SEQ ID NO: 5)
TTTCTTTGAAGCAGCAAGTATG
and
(SEQ ID NO: 6)
AGATGCTCTGAGAAAGGCATTA;
(iv)
(SEQ ID NO: 7)
CCAACCTCACCAACATTACAGAG
and
(SEQ ID NO: 8)
TCCAATGTCACATGAATGTAAATCAA;
(v)
(SEQ ID NO: 21)
CTCTTGAAGAATGAAAGCCTTGG
and
(SEQ ID NO: 22)
GTTTCTGTGTGCTTTATCCAGAACT.

### Acute Myeloid Leukemia (AML)

In certain embodiments, the kit includes at least one pair of primers capable of amplifying a region of a gene of interest from a sample to produce a double-stranded oligonucleotide. Suitable primer pairs for use herein include:

(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 30)
TTACATTTTTAATGCTCCTTTCTTTGA
and
(SEQ ID NO: 31)
GATGAGGTGATTTTCGTGGAAG;
(iv)
(SEQ ID NO: 32)
CTTGTCACCCACGGGAAAGT
and
(SEQ ID NO: 33)
AAAATAAGTAGGAAATAGCAGCCTCA;
(v)
(SEQ ID NO: 234)
AGTTTTACATTTTTAATGCTCCTTTCTT
and
(SEQ ID NO: 235)
TGATGAGGTGATTTTCGTGGA;
(vi)
(SEQ ID NO: 34)
TCTCTGTCCTCACAGAGTTCAAGC
and
(SEQ ID NO: 35)
CTTGGTCCAGCCAGGGACTA;
(vii)
(SEQ ID NO: 36)
CAAAAACATCCCACGCCTAGT
and
(SEQ ID NO: 37)
TGGGGTGAAGACCATTTTGAA;
(viii)
(SEQ ID NO: 238)
GCTGCAGTGGGACCACTATTATC;
and
(SEQ ID NO: 239)
GCTCCCGGAAGACAGTCCCC;
(ix)
(SEQ ID NO: 240)
CTGGCTGGACCAAGCCCATCA;
and
(SEQ ID NO: 241)
CTAGGCGAGGAGCTCCAGTCG;
(x)
(SEQ ID NO: 38)
GAGAAGAGGGTTGAGGAGTTCAA
and
(SEQ ID NO: 39)
GCAAAATCACATTATTGCCAACAT;
(xi)
(SEQ ID NO: 236)
GGCACGGTCTTCAGAGAAGC;
and
(SEQ ID NO: 237)
ACATTATTGCCAACATGACTTACTTGAT;
(xii)
(SEQ ID NO: 40)
CTAGTGCATTCAAGCACAATGG
and
(SEQ ID NO: 41)
CAGAGCCTAAACATCCCCTTAAA;
(xii)
(SEQ ID NO: 42)
AGGTGATCTATTTTTCCCTTTCTCC
and
(SEQ ID NO: 43)
TTATGTGTACCCAAAAAGGTGACA;
(xiv)
(SEQ ID NO: 44)
TGCCAGTTGTGCTTTTTGCT
and
(SEQ ID NO: 45)
ACAATAAAAGGCAGCTTGGACAC;
(xv)
(SEQ ID NO: 46)
ACTTGGCAGCCAGAAATATCCTC
and
(SEQ ID NO: 47)
GACTGTCAAGCAGAGAATGGGTACT;
(xvi)
(SEQ ID NO: 232)
GTATTCACAGAGACTTGGCAGC;
and
(SEQ ID NO: 233)
TGTCAAGCAGAGAATGGGTACT;
(xvii)
(SEQ ID NO: 48)
GATGTCTATGAAGTGTTGTGGTTCC
and
(SEQ ID NO: 49)
TGTTACAGAAATGAAATAAGACGGAAA;
(xviii)
(SEQ ID NO: 50)
GAGGCTCGCCATGCCGGGAG
and
(SEQ ID NO: 51)
GATGTCGATGGACGTCTCGTGC;
(xix)
(SEQ ID NO: 52)
CAGCGCCGCCTTCGGCTTTC
and
(SEQ ID NO: 53)
GCCCGGGTAGTCAAAGTCGC;
(xx)
(SEQ ID NO: 54)
GGCAGCAGGAGAAGGCCAAG
and
(SEQ ID NO: 55)
TTCATCCTCCTCGCGGGGCTC;
(xxi)
(SEQ ID NO: 56)
CGGCCGCTGGTGATCAAGC
and
(SEQ ID NO: 57)
GGTGACCGGGCTGCAGGTG;
(xxii)
(SEQ ID NO: 58)
CGCACTGCGGCCAGACCAC
and
(SEQ ID NO: 59)
GTTGCTGTTCTTGTCCACCGAC;
(xxiii)
(SEQ ID NO: 60)
GCGGGCAAGGCCAAGAAGTC
and
(SEQ ID NO: 61)
GTGTCCAGTTCGCGGCTCAG;
(xxiv)
(SEQ ID NO: 62)
GACCAGTGACAATGACCGCCTG
and
(SEQ ID NO: 63)
GGCGACCCCAAACCACTCC;

(xxv) a pair of primers having the reverse complement sequences of any of (i) through (xxiv); and
(xxvi) a pair of primers having at least 90% sequence identity to any of (i) through (xxv).

### Anemia (ANM) or Cytopenia

In certain embodiments, the kit includes at least one pair of primers capable of amplifying a region of a gene of interest from a sample to produce a double-stranded oligonucleotide. Suitable primer pairs for use herein include:

(i)
(SEQ ID NO: 64)
CCTTCCTCTTACTCTCTGCCTGC
and
(SEQ ID NO: 65)
GACAGCGGGCACACTTACCAG;
(ii)
(SEQ ID NO: 66)
CATTGTTAGGGCCGAGGCTAGA
and
(SEQ ID NO: 67)
ACGCACTTGTTTTACCTGTATGAGTC;
(iii)
(SEQ ID NO: 218)
GCCTCCCTTCCTCTTACTCTCT;
and
GCAGCCTGGGTAAGCACAC;
(iv)
(SEQ ID NO: 68)
GCAGATTCAACTTTCACGTATCAAACC
and
(SEQ ID NO: 69)
TGCAGAGCACGGGCTTTAATGT;
(v)
(SEQ ID NO: 70)
CCTCGCAGACATTAAAGCCCGT
and
(SEQ ID NO: 71)
CCACCATCACCACTGCTGCTG;
(vi)
(SEQ ID NO: 72)
CAGTGGTGATGGTGGTGAGGC
and
(SEQ ID NO: 73)
CCTAGCCCATCTGTGAGTCCAAC;
(vii)
(SEQ ID NO: 74)
TGTCCTCCCAAACCTCAGTAGC
and
(SEQ ID NO: 75)
AGCTTGGCCAGTTCCTTTCTCT;
(viii)
(SEQ ID NO: 76)
GTGTCTCGAGTATGTGCGGTCC
and
(SEQ ID NO: 77)
CATCGTGGGCTGGTGGAAGAA;
(ix)
(SEQ ID NO: 192)
CTTTCACGTATCAAACCACCC
and
(SEQ ID NO: 193)
CACGGGCTTTAATGTCTGC;
(x)
(SEQ ID NO: 194)
AAGATCCCAGATTCCCTACTG
and
(SEQ ID NO: 195)
GTGGGCTGGTGGAAGAAC;
(xi)
(SEQ ID NO: 196)
GGCGAGAGGTCACCACTG
and
(SEQ ID NO: 197)
CCATCACCACTGCTGCTG;
(xii)
(SEQ ID NO: 198)
AACTGAATGTGAGTCTGGCA
and
(SEQ ID NO: 199)
CACTAGAGACGGAATGGGAC;
(xiii)
(SEQ ID NO: 200)
CAGTGGTGATGGTGGTGAG
and
(SEQ ID NO: 201)
CCAACTGTAGCCCTCTGTAG;
(xiv)
(SEQ ID NO: 78)
TCCATTGCCTCTCCTTCTGTGC
and
(SEQ ID NO: 79)
TGCCAACTCCTTCATGCACCT;
(xv)
(SEQ ID NO: 80)
CCTAGGGGATGTTCCAGATGGC
and
(SEQ ID NO: 81)
AATGTGGGTTTGTTGCCATGAAAC;
(xvi)
(SEQ ID NO: 82)
TCTATCGTGTCCCCACAGGGAA
and
(SEQ ID NO: 83)
GGGGAAAGGTTGAACCCAAGGA;
(xvii)
(SEQ ID NO: 84)
CTTCACGCCGCCTTCCACCG
and
(SEQ ID NO: 85)
CTCCGGGCCAGTACCTTGAAA;
(xviii)
(SEQ ID NO: 204)
AGCAACGCCCATTTCACC
and
(SEQ ID NO: 205)
GCTCAGCTGCAAAGAATGTG;
(xix)
(SEQ ID NO: 206)
CAGGCAAGATGAGCGAGGC
and
(SEQ ID NO: 207)
GGGCCAGTACCTTGAAAGCG;
(xx)
(SEQ ID NO: 86)
TCCTGCTGTGTGGTTAGACGG
and
(SEQ ID NO: 87)
GAAGAGGTGGCGGATGACTGG;
(xxi)
(SEQ ID NO: 202)
CTGCTGTGTGGTTAGACGG
and
(SEQ ID NO: 203)
GAGGTGGCGGATGACTGG;
(xxii)
(SEQ ID NO: 88)
GCCCTGGGCATTCCTTCTTTATT
and
(SEQ ID NO: 89)
ACTTCTAAGATGTGGCAAGATGGC;
(xxiii)
(SEQ ID NO: 90)
GTTGGGGCATAGTTAAAACCTGTGT
and
(SEQ ID NO: 91)
TTCCTCTGTGTTGGCGGATACC;
(xxiv)
(SEQ ID NO: 92)
GGTATCCGCCAACACAGAGGAA
and
(SEQ ID NO: 93)
AGGAGACTGGAATTCTCGAATAAGGA;
(xxv)
(SEQ ID NO: 208)
GGCTGCTGGTCTGGCTACTAT
and
(SEQ ID NO: 209)
CCAGGGCAGAGGCTACAAC;
(xxvi)
(SEQ ID NO: 210)
TGTAGGTCTTGTGGATGAGC
and
(SEQ ID NO: 211)
TTCCTCTGTGTTGGCGGATA;
(xxvii)
(SEQ ID NO: 212)
CAGAGGAAAGGTAAATCCACCA
and
(SEQ ID NO: 213)
GGAATAAGATACCCAATAGCCTTCA;
(xxviii)
(SEQ ID NO: 214)
TTCCTTCTTTATTGCCCTTCTTA
and
(SEQ ID NO: 215)
CAACTTACCATGTTCAATGATTTCAA;

(xxix) a pair of primers having the reverse complement sequences of any of (i) through (xxviii); and
(xxx) a pair of primers having at least 90% sequence identity to any of (i) through (xxix).

### Chronic Lymphocytic Leukemia (CLL)

In certain embodiments, the kit includes at least one pair of primers capable of amplifying a region of a gene of interest from a sample to produce a double-stranded oligonucleotide. Suitable primer pairs for use herein include:

(i)
(SEQ ID NO: 88)
GCCCTGGGCATTCCTTCTTTATT
and
(SEQ ID NO: 89)
ACTTCTAAGATGTGGCAAGATGGC;
(ii)
(SEQ ID NO: 90)
GTTGGGGCATAGTTAAAACCTGTGT
and
(SEQ ID NO: 91)
TTCCTCTGTGTTGGCGGATACC;
(iii)
(SEQ ID NO: 92)
GGTATCCGCCAACACAGAGGAA
and
(SEQ ID NO: 93)
AGGAGACTGGAATTCTCGAATAAGGA;
(iv)
(SEQ ID NO: 208)
GGCTGCTGGTCTGGCTACTAT
and
(SEQ ID NO: 209)
CCAGGGCAGAGGCTACAAC;
(v)
(SEQ ID NO: 210)
TGTAGGTCTTGTGGATGAGC
and
(SEQ ID NO: 211)
TTCCTCTGTGTTGGCGGATA;
(vi)
(SEQ ID NO: 212)
CAGAGGAAAGGTAAATCCACCA
and
(SEQ ID NO: 213)
GGAATAAGATACCCAATAGCCTTCA;
(vii)
(SEQ ID NO: 214)
TTCCTTCTTTATTGCCCTTCTTA
and
(SEQ ID NO: 215)
CAACTTACCATGTTCAATGATTTCAA;
(viii)
(SEQ ID NO: 230)
CTACTCCTCGCCTGTGGACAAC
and
(SEQ ID NO: 231)
GATCTGGGACTGCATGCTGGTG;
(ix)
(SEQ ID NO: 220)
CCAACGTCAGTGAGGCAGAT
and
(SEQ ID NO: 221)
GGATGACAATACCAGGCAGGA;
(x)
(SEQ ID NO: 222)
TGCTTTCCTTGGAGCCAAAT
and
(SEQ ID NO: 223)
TGTGTTAGCTGGAGTGAAAACTTG;
(xi)
(SEQ ID NO: 224)
ATATGCCTGAGCGTTTCGATGC
and
(SEQ ID NO: 225)
GTGGCCTTCTAGCCAACCTCT;
(xii)
(SEQ ID NO: 226)
CCCAGGGGATATGCTGAACTA
and
(SEQ ID NO: 227)
ACCTGGAGAGAGGCTGAGTG;
(xiii)
(SEQ ID NO: 228)
TTGGCTTGCAGGTGCCCATC
and
(SEQ ID NO: 229)
GGCGAGTCCAGAACCAAGATTT;

### BCR/ABL1-Induced Leukemogenesis

In certain embodiments, the kit includes at least one primer (e.g., at least one primer pair) capable of amplifying a region of a gene of interest from a sample to produce a double-stranded oligonucleotide. A suitable primer for use herein includes:

(i)
(SEQ ID NO: 243)
ATGCTGACCAACTCGTGTGTGAA;
(ii)
(SEQ ID NO: 244)
CAGAACTCGCAACAGTCCTTCGA;
(iii)
(SEQ ID NO: 245)
GTGCGTGGAGGAGATCGAGCG;
(iv)
(SEQ ID NO: 246)
TGATTATAGCCTAAGACCCGGAGC;
(v)
(SEQ ID NO: 247)
CCTAAGACCCGGAGCTTTTCAC;

(vi) two or more primers having the reverse complement sequences of any of (i) through (v); and
(vii) two or more primers having at least 90% sequence identity to any of (i) through (vi). In certain embodiments, the kit can include a pair of primers having the reverse complement sequences of any of (i) through (iv), (xxvi), (xxx), (xv), and/or (vii). In certain embodiments, the kit can include a pair of primers having at least 80%, at least 85%, at least 90%, at least 95%, at least 96%, at least 97%, at least 98%, or at least 99% sequence identity to any of the above primer pairs.

In certain embodiments, the kit can include at least 2, 3, or 4 pairs of primers.

In certain embodiments, at least one of the pairs of primers comprises a tag. In certain embodiments, the tag comprises a sequencing primer such as an M13 primer. An M13 primer can include, for example, a M13 forward sequence or an M13 reverse sequence (e.g., AGGAAACAGCTATGACCAT (SEQ ID NO: 187) or TGTAAAACGACGGCCAGT (SEQ ID NO: 188)), or a complement or reverse complement thereof. In certain embodiments, the tag comprises a basic CG clamp sequence (e.g., GCGTCCCG (SEQ ID NO: 189): GCCCCCGCCG (SEQ ID NO: 190); or GCGGCCCGCCGCCCCCGCCG (SEQ ID NO: 191)).

The kit can also include one or more reagents suitable for performing High Resolution Melting (HRM) analysis including, for example, a thermostable DNA polymerase: a mix of deoxynucleoside triphosphates which is usually dA, dG, dC and dT, or dA, dG, dC and dU: a buffer (including, e.g., magnesium), and a fluorescent, double stranded DNA binding dye.

In certain embodiments, the kit can further comprise a wild-type or mutant (i.e., positive) control. In certain embodiments, the wild-type control is wild-type DNA from the region of the gene of interest (e.g., wild-type DNA from exon 10 of the MPL gene, from exon 9 of the CALR gene, or from exon 12 or 14 of the JAK2 gene). In certain embodiments, the mutant control is DNA comprising a mutation in the region of the gene of interest, for example, a 1544G>T mutation in exon 10 of the MPL gene, a 1543_1544TG>AA mutation in exon 10 of the MPL gene, a 1092_1143del deletion in exon 9 of the CALR gene, a 1154_1155insTTGCC insertion in exon 9 of the CALR gene: a 1849G>T mutation in exon 14 of the JAK2 gene, a 1611_1616delTCACAA deletion in exon 12 of the JAK2 gene, a 1624_1629delAATGAA deletion in exon 12 of the JAK2 gene, or a 1615_1616delAAinsTT deletion/insertion in exon 12 of the JAK2 gene.

The kit may also include a dye, such as SYTO® 9, SYBR® Green, or Chai Green™, or any modification thereof, such as the MeltDoctor™ HRM Dye, a stabilized form of SYTO® 9, available from Thermo Fisher (Waltham, MA). Other dyes suitable for inclusion in a kit are SYTO™ dyes (SYTO™ 11, SYTO™ 12, SYTO™ 13, SYTO™ 14, SYTO™ 15, SYTO™ 16, SYTO™ 18, SYTO™ 20, SYTO™ 21, SYTO™ 22, SYTO™ 23, SYTO™ 24, SYTO™ 25, SYTO™ BC, Molecular Probes, Eugene, OR), EvaGreen® (Biotium, Fremont, CA), and those disclosed dyes in WO 2008/052742.

Exemplary DNA polymerases suitable for use herein include any thermostable DNA polymerase suitable for amplifying DNA in vitro such as Taq polymerase or a modified version thereof (e.g., AmpliTaq Gold® 360 DNA Polymerase, Hot Start Taq 2X Master Mix (New England BioLabs), or MeltDoctor™ HRM Master Mix).

For example, in some embodiments, a 1X dilution of Hot Start Taq 2X Master Mix comprises 10 mM Tris-HCl, 50 mM KCl, 1.5 mM MgCl₂, 0.2 mM dNTPs, 5% Glycerol, and 25 units/mL Hot Start Taq DNA polymerase, at pH 8.3 (@ 25° C.

In some embodiments, a 2X mix of MeltDoctor™ HRM Master Mix comprises AmpliTaq Gold™ 360 DNA Polymerase, MeltDoctor™ HRM Dye, a dNTP blend including dUTP, magnesium salts, and other buffer components.

Any suitable Master Mix may be used.

### EXAMPLES

In order that the invention described herein can be more fully understood, the following examples are set forth. It should be understood that these examples are included merely for purposes of illustration of certain aspects and embodiments of the present invention, and are not to be construed as limiting the invention in any manner.

### Example 1: Detection of Mutations in MPL, CALR, and JAK2 in Cell Line Samples Using Amplification and High Resolution Melting

In this example, mutations in MPL Exon 10, CALR Exon 9, JAK2 Exon 12, and JAK2 Exon 14 were detected using the primers and methods described herein.

A Master Mix was prepared in a 1.7 mL tube for each of the four targets containing, per sample to be measured, 10 μL 2X MeltDoctor™ HRM Master Mix (Thermo Fisher, Waltham, MA) and 8 μL Primer Mix (containing forward and reverse primer for each of the four targets).

Each Master Mix tube was capped and vortexed for about 3-5 sec, briefly centrifuged and stored on ice until use.

A MicroAmp Optical 96-Well Reaction Plate was labeled with appropriate sample information and 18 μL of reaction mix was aliquoted to the relevant wells of a MicroAmp Optical 96-Well Reaction Plate. 2 μL of each DNA sample, control(s), or water (NTC) was added to appropriate wells.

Separate pipette tips were used for each sample to avoid cross-contamination of the samples. Tubes were pipette-mixed to ensure sample was well mixed into reaction mixture. MicroAmp Optical Film (Thermo Fisher Scientific, Waltham, MA) was placed over plates and sealed. Plates were spun briefly to eliminate any air bubbles from the solution.

An ABI 7500 Real-Time PCR Instrument with 7500 Software v2.3 (Thermo Fisher Scientific/Applied Biosystems™, Waltham MA) was used to perform PCR amplification and HRM analysis according to the parameters shown in TABLE 22.

TABLE 22

| | | | | | Ramp |
|---|---|---|---|---|---|
| Stage | Step | Temp | Time | | rate |
| Holding | Enzyme activation | 95° C. | 10 | min | 100% |
| Cycling | Denature | 95° C. | 15 | sec | 100% |
| (40 cycles) | Anneal/extend | 60° C. | 1 | min | 100% |
| Melt curve/ | Denature | 95° C. | 10 | sec | 100% |
| dissociation | Anneal | 60° C. | 1 | min | 100% |
| | High resolution | 95° C. | 15 | sec | 1% |
| | melting | | | | |
| | Anneal | 60° C. | 15 | sec | 100% |

Amplification plots, melt curves, and difference plots were generated. Amplification plots were analyzed for outliers (i.e., wells having CT values that differ from replicates by more than 2). Melt curves were reviewed for unexpected peaks and then analyzed using High Resolution Melt Software v3.1. Pre- and post-melt regions were automatically set by the software. Derivative Melt Curves were generated and pre- and post-melt regions were adjusted as necessary to optimize separation and variant calls. The software was then used to generate data showing which samples contained variant calls, indicating that mutations were detected in these samples.

Cell lines transformed with a plasmid harboring a mutations in one of the four mutations to produce cells having 5%, 10%, 15%, or 20% allelic frequency of the mutation were the samples tested. Mutations were MPL W515L (c.1544G>T), CALR L367fs*46 (c.1092_1143del52), JAK2 V617F (c.1849G>T), and JAK2 N542_E543del (c.1624_1629delAATGAA). Untransformed cell lines (shown as “WT” in TABLE 23) and cell lines transformed with plasmids harboring the known mutations were analyzed and the results (PDX Results) shown in TABLE 23.

TABLE 23

| | Controls Manufacturer (Horizon) | | | | PDX Results | | | |
|---|---|---|---|---|---|---|---|---|
| | JAK2 | | | | JAK2 | | | |
| | E14 | JAK2 | | | E14 | JAK2 | | |
| Case Number | (V617F) | E12 | MPL | CALR | (V617F) | E12 | MPL | CALR |
| JAK2_E14 Cell | D | ND | ND | ND | D | ND | ND | ND |
| Line #1 (5%) | | | | | | | | |
| JAK2_E14 Cell | D | ND | ND | ND | D | ND | ND | ND |
| Line #2 (10%) | | | | | | | | |
| JAK2_E14 Cell | D | ND | ND | ND | D | ND | ND | ND |
| Line#3 (15%) | | | | | | | | |
| JAK2_E14 Cell | D | ND | ND | ND | D | ND | ND | ND |
| Line#4 (20%) | | | | | | | | |
| JAK2_E14 Cell | ND | ND | ND | ND | ND | ND | ND | ND |
| Line#5 (WT) | | | | | | | | |
| JAK2_E12 Cell | ND | D | ND | ND | ND | D | ND | ND |
| Line#1 (5%) | | | | | | | | |
| JAK2_E12 Cell | ND | D | ND | ND | ND | D | ND | ND |
| Line#2 (10%) | | | | | | | | |
| JAK2_E12 Cell | ND | D | ND | ND | ND | D | ND | ND |
| Line#3 (15%) | | | | | | | | |
| JAK2_E12 Cell | ND | D | ND | ND | ND | D | ND | ND |
| Line#4 (20%) | | | | | | | | |
| JAK2_E12 Cell | ND | ND | ND | ND | ND | ND | ND | ND |
| Line#5 (WT) | | | | | | | | |
| MPL Cell | ND | ND | D | ND | ND | ND | D | ND |
| Line#1 (5%) | | | | | | | | |
| MPL Cell | ND | ND | D | ND | ND | ND | D | ND |
| Line#2 (10%) | | | | | | | | |
| MPL Cell | ND | ND | D | ND | ND | ND | D | ND |
| Line#3 (15%) | | | | | | | | |
| MPL Cell | ND | ND | D | ND | ND | ND | D | ND |
| Line#4 (20%) | | | | | | | | |
| MPL Cell | ND | ND | ND | ND | ND | ND | ND | ND |
| Line#5 (WT) | | | | | | | | |
| CALR Cell | ND | ND | ND | D | ND | ND | ND | D |
| Line#1 (5%) | | | | | | | | |
| CALR Cell | ND | ND | ND | D | ND | ND | ND | D |
| Line#2 (10%) | | | | | | | | |
| CALR Cell | ND | ND | ND | D | ND | ND | ND | D |
| Line#3 (15%) | | | | | | | | |
| CALR Cell | ND | ND | ND | D | ND | ND | ND | D |
| Line#4 (20%) | | | | | | | | |
| CALR Cell | ND | ND | ND | ND | ND | ND | ND | ND |
| Line#5 (WT) | | | | | | | | |

D = mutation detected
ND = mutation not detected

As shown in TABLE 23, the detection methods disclosed herein detected the presence or absence of mutations in a biological sample having a known genotype.

### Example 2: Detection of Mutations in MPL, CALR, and JAK2 in Samples of Unknown Genotype Using Amplification and High Resolution Melting

Next, human DNA samples that previously had been assessed using Sanger sequencing (for JAK2 E12) or RT-PCR (JAK2 E14 (V617F), MPL, and CALR) (collectively, “Reference Lab Result”) were analyzed using the method of Example 1 and the data shown in TABLE 24.

TABLE 24

| | | Reference Lab Result | | | | PDX Results | | | |
|---|---|---|---|---|---|---|---|---|---|
| | | JAK2 | | | | JAK2 | | | |
| S: | Case | E14 | JAK2 | | | E14 | JAK2 | | |
| No | Number | (V617F) | E12 | MPL | CALR | (V617F) | E12 | MPL | CALR |
| 1 | P18-00737 | D | NP | NP | NP | D | ND | ND | ND |
| 2 | P18-00821 | D | NP | NP | NP | D | ND | ND | ND |
| 3 | P18-00871 | D | NP | NP | NP | D | ND | ND | ND |
| 4 | P18-00879 | ND | ND | NP | NP | ND | ND | ND | ND |
| 5 | P18-00883 | ND | ND | ND | D | ND | ND | ND | D |
| 6 | P18-00889 | ND | ND | ND | ND | ND | ND | ND | ND |
| 7 | P18-00907 | ND | ND | ND | ND | ND | ND | ND | ND |
| 8 | P18-00924 | ND | ND | ND | ND | ND | ND | ND | ND |
| 9 | P18-00927 | ND | ND | ND | ND | ND | ND | ND | ND |
| 10 | P18-00941 | ND | ND | ND | ND | ND | ND | ND | ND |
| 11 | P18-00943 | ND | ND | NP | NP | ND | ND | ND | ND |
| 12 | P18-00956 | ND | ND | NP | NP | ND | ND | ND | ND |
| 13 | P18-01021 | ND | ND | ND | ND | ND | ND | ND | ND |
| 14 | P18-01042 | ND | ND | NP | NP | ND | ND | ND | ND |
| 15 | P18-01047 | ND | ND | ND | ND | ND | ND | ND | ND |

D = mutation detected
ND = mutation not detected
NP = not performed

As shown in TABLE 24, the detection methods disclosed herein detected the presence or absence of mutations in a human DNA sample of unknown mutation status with the same accuracy as Sanger sequencing or RT-PCR (with the caveat that, in this experiment, no positive control existed for JAK2 E12 or MPL).

### Example 3: Detection of BCR/ABL Isoforms in Samples of Unknown Genotype Using Amplification and High Resolution Melting

BCR/ABL1 is a tumor marker for Philadelphia (Ph) chromosome-positive leukemia. The status of the BCR/ABL1 fusion leads to informed prognosis and clinical management decisions and serves as a paradigm for molecularly-targeted cancer therapy.

The BCR/ABL1 fusion gene results from the usually reciprocal chromosomal translocation t (9;22) (q34;q11) (Philadelphia chromosome) of two genes, ABL on chromosome 9 (a cellular proto-oncogene coding for a tyrosine kinase), and BCR on chromosome 22 (which codes for a serine kinase). In leukemia cells, the Ph chromosome impairs the survival and proliferation signaling pathways and disrupts genomic stability. The Ph chromosome is a hallmark of CML and is associated with 95% of CML cases and approximately 25% of adult ALL cases and less frequently in other hematological diseases.

Variations of four common BCR/ABL1 isoforms include p210, p190, p230, and p203. Each isoform is produced by a fusion of the BCR and ABL genes but differs in the location of the breakpoint, which determines which BCR/ABL1 protein isoforms are expressed, and are linked to different disease phenotypes. This example describes an assay that can detect four variants of isoforms p210 and p203 between BCR exons 13 and 14 (b2 and b3) and ABL exons 2 and 3 (a2 and a3), two variants of isoform p190 between BCR exons 1 and ABL exons 1 and 2 (e1 and a2) and exon 3 (a3), and two variants of isoform p230 between BCR exon 19 (e19) and ABL exons 2 and 3 (a2 and a3) (FIG. 1). The assay describes the results of a melt curve, using the three assays described in FIG. 1 and primers described herein (SEQ ID NOs: 243-247), to detect BCR/ABL1 isoforms p210, p190, p230, and p203.

### Results

The presence of a BCR/ABL1 fusion variant is indicated through amplification of target sequences in the presence of high fluorescence dsDNA-binding dye and melt curve analysis. Forward and reverse primers hybridized to specific sequences and only amplified in the presence of a fusion product. If no fusion product existed, no production of a large fusion product occurred. Immediately after amplification, melt curve analysis measured the fluorescence of each product in the reaction as the temperature increases from 60° C. to 90° C. and indicated if a product was amplified and identify the melting temperature (TM) of each product. Each assay contained an amplification control to confirm that each reaction occurred as expected and interpretation of the fluorescent signal would indicate the presence or absence of a fusion.

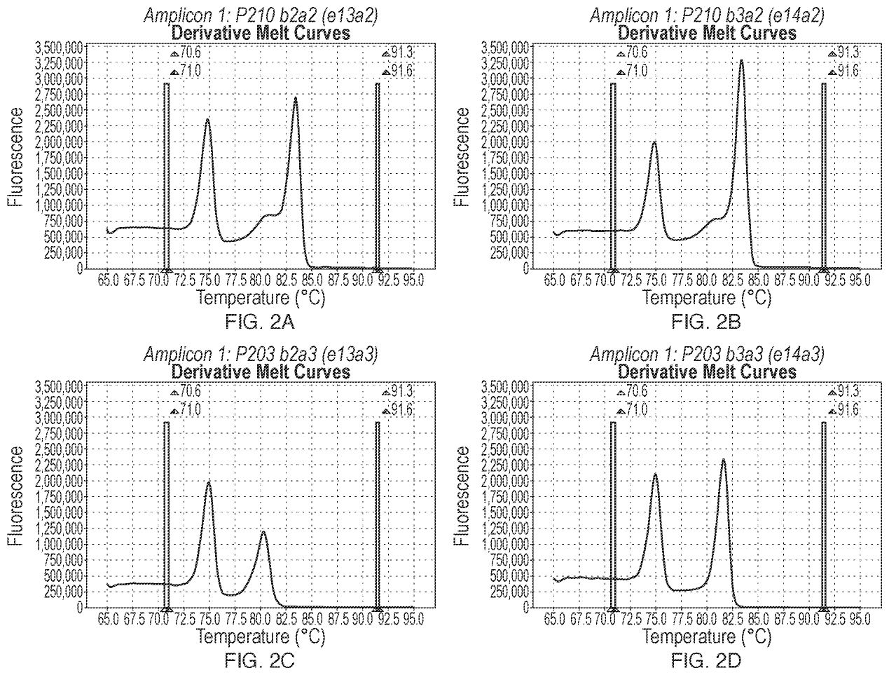

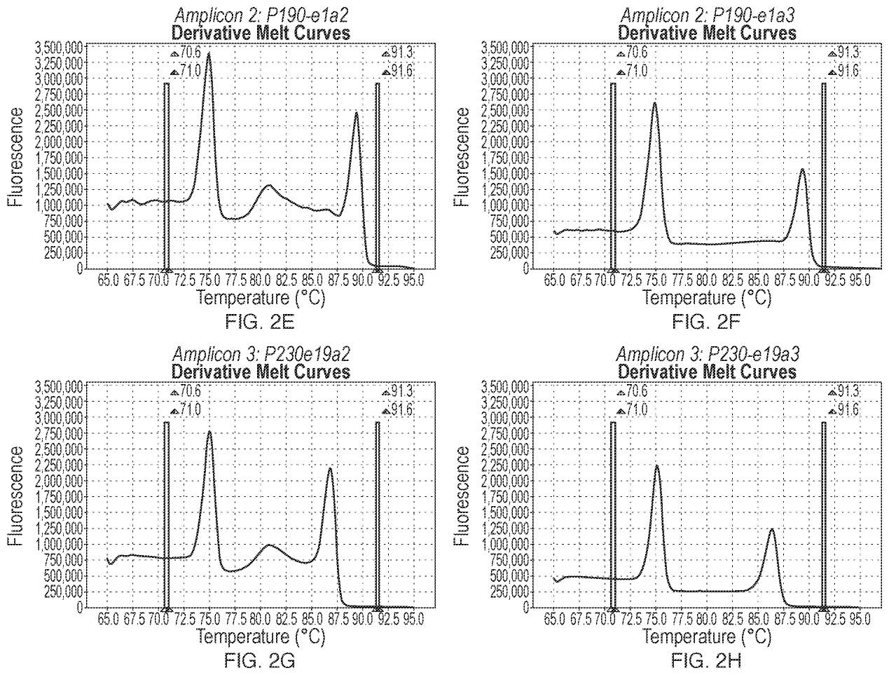

Samples were analyzed using the method generally described in Example 1. FIGS. 2A-2H show exemplary melt curve signatures for the BCR/ABL1 isoforms p210 (b2a2 and b3a2), p203 (b2a3 and b3a3), p190 (ela2 and ela3), and p230 (e19a2 and e19a3). The melt curve raw data were plotted as fluorescence vs. temperature. As a product melted, the fluorescence decreased rapidly. The derivative plots of FIG. 2A-2H showed changes in fluorescence at given temperatures. Large changes in fluorescence appeared as peaks and indicated the presence and melting temperature (Tm) of the products. The amplification controls produced a single peak at Tm≤75° C. For negative cases, only the single peak of the control amplicon was visible. Positive cases exhibited the control peak and a second peak for a fusion product.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed:

1. A method for determining the presence or absence of a mutation in a gene of interest, the method comprising:
(a) amplifying a region of a gene of interest from a sample to produce a double-stranded oligonucleotide, wherein the amplifying step comprises performing PCR using a pair of primers, selected from the group consisting of:
(i)
(SEQ ID NO: 1)
TAGCCTGGATCTCCTTGGTG
and
(SEQ ID NO: 2)
GCGGTACCTGTAGTGTGCAG;
(ii)
(SEQ ID NO: 3)
GCAGAGAAACAAATGAAGGACAAAC
and
(SEQ ID NO: 4)
CTTCCTCCTTGTCCTCCTCA;
(iii)
(SEQ ID NO: 5)
TTTCTTTGAAGCAGCAAGTATG
and
(SEQ ID NO: 6)
AGATGCTCTGAGAAAGGCATTA;
(iv)
(SEQ ID NO: 7)
CCAACCTCACCAACATTACAGAG
and
(SEQ ID NO: 8)
TCCAATGTCACATGAATGTAAATCAA;
(v)
(SEQ ID NO: 21)
CTCTTGAAGAATGAAAGCCTTGG
and
(SEQ ID NO: 22)
GTTTCTGTGTGCTTTATCCAGAACT;
(vi) a pair of primers having the reverse complement sequences of any of (i) through (v); and
(vii) a pair of primers having at least 90% sequence identity to any of (i) through (vi);
(b) determining that the double-stranded oligonucleotide comprises or does not comprise the mutation in the gene of interest by means of a High Resolution Melting (HRM) analysis.

2. The method of claim 1, further comprising the step of sequencing the mutation.

3. The method of claim 1 or claim 2, wherein the sample is from a subject in need of treatment of a myeloid malignancy, and the method further comprises treating the subject with an effective amount of a therapeutic agent to treat the myeloid malignancy.

4. The method of any one of claims 1-3, wherein the determining step comprises comparing HRM analysis results of the double-stranded oligonucleotide to a control.

5. The method of claim 4, wherein the control comprises HRM analysis results of a wild-type sample.

6. The method of claim 4, wherein the control comprises HRM analysis results of a sample comprising the mutation of interest.

7. The method of any one of claims 1-6, wherein the HRM analysis is performed using a dye.

8. The method of claim 7, wherein the dye comprises SYTO® 9, SYBR® Green, or Chai Green™.

9. The method of any one of claims 1-8, wherein the amplifying step comprises performing PCR using, in parallel, at least 2 pairs of primers from (i) through (vi).

10. The method of any one of claims 1-9, wherein the amplifying step comprises performing PCR using, in parallel, at least 3 pairs of primers from (i) through (vi).

11. The method of any one of claims 1-10, wherein the amplifying step comprises performing PCR using, in parallel, at least 4 pairs of primers from (i) through (vi).

12. The method of any one of claims 1-11, wherein the gene of interest comprises MPL, and wherein the pair of primers are selected from the list consisting of:
(i)
(SEQ ID NO: 1)
TAGCCTGGATCTCCTTGGTG
and
(SEQ ID NO: 2)
GCGGTACCTGTAGTGTGCAG;
(ii) a pair of primers having the reverse complement sequences of (i); and
(iii) a pair of primers having at least 90% sequence identity to any of (i) through (ii).

13. The method of any one of claims 1-11, wherein the gene of interest comprises CALR, and wherein the pair of primers are selected from the list consisting of:
(i)
(SEQ ID NO: 3)
GCAGAGAAACAAATGAAGGACAAAC
and
(SEQ ID NO: 4)
CTTCCTCCTTGTCCTCCTCA;
(ii) a pair of primers having the reverse complement sequences of (i); and
(iii) a pair of primers having at least 90% sequence identity to any of (i) through (ii).

14. The method of any one of claims 1-11, wherein the gene of interest comprises JAK2, and wherein the pair of primers are selected from the list consisting of:
(i)
(SEQ ID NO: 5)
TTTCTTTGAAGCAGCAAGTATG
and
(SEQ ID NO: 6)
AGATGCTCTGAGAAAGGCATTA;
(ii)
(SEQ ID NO: 7)
CCAACCTCACCAACATTACAGAG
and
(SEQ ID NO: 8)
TCCAATGTCACATGAATGTAAATCAA;
(iii)
(SEQ ID NO: 21)
CTCTTGAAGAATGAAAGCCTTGG
and
(SEQ ID NO: 22)
GTTTCTGTGTGCTTTATCCAGAACT;
(iv) a pair of primers having the reverse complement sequences of any of (i) through (iii); and
(v) a pair of primers having at least 90% sequence identity to any of (i) through (iv).

15. The method of any one of claims 1-14, wherein at least one of the pair of primers comprises a tag.

16. The method of claim 15, wherein the tag comprises M13 or a basic GC clamp sequence.

17. A method of treating a subject in need of treatment of a myeloid malignancy, the method comprising:
(a) amplifying a region of a gene of interest from a sample from the subject to produce a double-stranded oligonucleotide, wherein the amplifying step comprises performing PCR using a pair of primers, selected from the group consisting of:
(i)
(SEQ ID NO: 1)
TAGCCTGGATCTCCTTGGTG
and
(SEQ ID NO: 2)
GCGGTACCTGTAGTGTGCAG;
(ii)
(SEQ ID NO: 3)
GCAGAGAAACAAATGAAGGACAAAC
and
(SEQ ID NO: 4)
CTTCCTCCTTGTCCTCCTCA;
(iii)
(SEQ ID NO: 5)
TTTCTTTGAAGCAGCAAGTATG
and
(SEQ ID NO: 6)
AGATGCTCTGAGAAAGGCATTA;
(iv)
(SEQ ID NO: 7)
CCAACCTCACCAACATTACAGAG
and
(SEQ ID NO: 8)
TCCAATGTCACATGAATGTAAATCAA;
(v)
(SEQ ID NO: 21)
CTCTTGAAGAATGAAAGCCTTGG
and
(SEQ ID NO: 22)
GTTTCTGTGTGCTTTATCCAGAACT;
(vi) a pair of primers having the reverse complement sequences of any of (i) through (v); and
(vii) a pair of primers having at least 90% sequence identity to any of (i) through (vi):
(b) determining that the double-stranded oligonucleotide comprises a mutation in the gene of interest by means of a High Resolution Melting (HRM) analysis;
(c) sequencing the mutation;
(d) treating the subject in need of treatment of the myeloid malignancy with an effective amount of a therapeutic agent.

18. The method of claim 17, wherein the determining step comprises comparing HRM analysis results of the double-stranded oligonucleotide to a control.

19. The method of claim 18, wherein the control comprises HRM analysis results of a wild-type sample.

20. The method of claim 18, wherein the control comprises HRM analysis results of a sample comprising the mutation of interest.

21. The method of any of claims 17-20, wherein the HRM analysis is performed using a dye.

22. The method of claim 21, wherein the dye comprises SYTO® 9, SYBR® Green, or Chai Green™.

23. The method of any one of claims 17-22, wherein the amplifying step comprises performing PCR using, in parallel, at least 2 pairs of primers from (i) through (vi).

24. The method of any one of claims 17-22, wherein the amplifying step comprises performing PCR using, in parallel, at least 3 pairs of primers from (i) through (vi).

25. The method of any one of claims 17-22, wherein the amplifying step comprises performing PCR using, in parallel, at least 4 pairs of primers from (i) through (vi).

26. The method of any one of claims 17-25, wherein the gene of interest comprises MPL, and wherein the pair of primers are selected from the list consisting of:
(i)
(SEQ ID NO: 1)
TAGCCTGGATCTCCTTGGTG
and
(SEQ ID NO: 2)
GCGGTACCTGTAGTGTGCAG;
(ii) a pair of primers having the reverse complement sequences of (i); and
(iii) a pair of primers having at least 90% sequence identity to any of (i) through (ii).

27. The method of any one of claims 17-25, wherein the gene of interest comprises CALR, and wherein the pair of primers are selected from the list consisting of:
(i)
(SEQ ID NO: 3)
GCAGAGAAACAAATGAAGGACAAAC
and
(SEQ ID NO: 4)
CTTCCTCCTTGTCCTCCTCA;
(ii) a pair of primers having the reverse complement sequences of (i); and
(iii) a pair of primers having at least 90% sequence identity to any of (i) through (ii).

28. The method of any one of claims 17-25, wherein the gene of interest comprises JAK2, and wherein the pair of primers are selected from the list consisting of:
(i)
(SEQ ID NO: 5)
TTTCTTTGAAGCAGCAAGTATG
and
(SEQ ID NO: 6)
AGATGCTCTGAGAAAGGCATTA;
(ii)
(SEQ ID NO: 7)
CCAACCTCACCAACATTACAGAG
and
(SEQ ID NO: 8)
TCCAATGTCACATGAATGTAAATCAA;
(iii)
(SEQ ID NO: 21)
CTCTTGAAGAATGAAAGCCTTGG
and
(SEQ ID NO: 22)
GTTTCTGTGTGCTTTATCCAGAACT;
(iv) a pair of primers having the reverse complement sequences of any of (i) through (iii); and
(v) a pair of primers having at least 90% sequence identity to any of (i) through (iv).

29. The method of any one of claims 17-28, wherein at least one of the pair of primers comprises a tag.

30. The method of claim 29, wherein the tag comprises M13 or a basic GC clamp sequence.

31. A kit comprising:
(a) a pair of primers capable of amplifying a region of a gene of interest from a sample to produce a double-stranded oligonucleotide, wherein the pair of primers is selected from the group consisting of:
(i)
(SEQ ID NO: 1)
TAGCCTGGATCTCCTTGGTG
and
(SEQ ID NO: 2)
GCGGTACCTGTAGTGTGCAG;
(ii)
(SEQ ID NO: 3)
GCAGAGAAACAAATGAAGGACAAAC
and
(SEQ ID NO: 4)
CTTCCTCCTTGTCCTCCTCA;
(iii)
(SEQ ID NO: 5)
TTTCTTTGAAGCAGCAAGTATG
and
(SEQ ID NO: 6)
AGATGCTCTGAGAAAGGCATTA;
(iv)
(SEQ ID NO: 7)
CCAACCTCACCAACATTACAGAG
and
(SEQ ID NO: 8)
TCCAATGTCACATGAATGTAAATCAA;
(v)
(SEQ ID NO: 21)
CTCTTGAAGAATGAAAGCCTTGG
and
(SEQ ID NO: 22)
GTTTCTGTGTGCTTTATCCAGAACT;
(vi) a pair of primers having the reverse complement sequences of any of (i) through (v); and
(vii) a pair of primers having at least 90% sequence identity to any of (i) through (vi);
(b) one or more reagents suitable for performing High Resolution Melting (HRM) analysis.

32. The kit of claim 31, wherein the kit comprises at least 2 pairs of primers from (i) through (vii).

33. The kit of claim 31, wherein the kit comprises at least 3 pairs of primers from (i) through (vii).

34. The kit of claim 31, wherein the kit comprises at least 4 pairs of primers from (i) through (vii).

35. The kit of any of claims 31-34, further comprising a wild-type control.

36. The kit of any of claims 31-34, further comprising a mutant control.

37. The kit of any of claims 31-36, further comprising a dye.

38. The kit of claim 37, wherein the dye comprises SYTO® 9, SYBR® Green, or Chai Green™.

39. The kit of any one of claims 31-38, wherein the gene of interest comprises MPL, and wherein the pair of primers are selected from the list consisting of:
(i)
(SEQ ID NO: 1)
TAGCCTGGATCTCCTTGGTG
and
(SEQ ID NO: 2)
GCGGTACCTGTAGTGTGCAG;
(ii) a pair of primers having the reverse complement sequences of (i); and
(iii) a pair of primers having at least 90% sequence identity to any of (i) through (ii).

40. The kit of any one of claims 31-38, wherein the gene of interest comprises CALR, and wherein the pair of primers are selected from the list consisting of:
(i)
(SEQ ID NO: 3)
GCAGAGAAACAAATGAAGGACAAAC
and
(SEQ ID NO: 4)
CTTCCTCCTTGTCCTCCTCA;
(ii) a pair of primers having the reverse complement sequences of (i); and
(iii) a pair of primers having at least 90% sequence identity to any of (i) through (ii).

41. The kit of any one of claims 31-38, wherein the gene of interest comprises JAK2, and wherein the pair of primers are selected from the list consisting of:
(i)
(SEQ ID NO: 5)
TTTCTTTGAAGCAGCAAGTATG
and
(SEQ ID NO: 6)
AGATGCTCTGAGAAAGGCATTA;
(ii)
(SEQ ID NO: 7)
CCAACCTCACCAACATTACAGAG
and
(SEQ ID NO: 8)
TCCAATGTCACATGAATGTAAATCAA;
(iii)
(SEQ ID NO: 21)
CTCTTGAAGAATGAAAGCCTTGG
and
(SEQ ID NO: 22)
GTTTCTGTGTGCTTTATCCAGAACT;
(iv) a pair of primers having the reverse complement sequences of any of (i) through (iii); and
(v) a pair of primers having at least 90% sequence identity to any of (i) through (iv).

42. The kit of any one of claim 31-41, wherein at least one of the pair of primers comprises a tag.

43. The kit of claim 42, wherein the tag comprises M13 or a basic GC clamp sequence.

44. A method for determining the presence or absence of a mutation in a gene of interest, the method comprising:
(a) amplifying a region of a gene of interest from a sample to produce a double-stranded oligonucleotide, wherein the amplifying step comprises performing PCR using a pair of primers, selected from the group consisting of:
(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 30)
TTACATTTTTAATGCTCCTTTCTTTGA
and
(SEQ ID NO: 31)
GATGAGGTGATTTTCGTGGAAG;
(iv)
(SEQ ID NO: 32)
CTTGTCACCCACGGGAAAGT
and
(SEQ ID NO: 33)
AAAATAAGTAGGAAATAGCAGCCTCA;
(v)
(SEQ ID NO: 234)
AGTTTTACATTTTTAATGCTCCTTTCTT
and
(SEQ ID NO: 235)
TGATGAGGTGATTTTCGTGGA;
(vi)
(SEQ ID NO: 34)
TCTCTGTCCTCACAGAGTTCAAGC
and
(SEQ ID NO: 35)
CTTGGTCCAGCCAGGGACTA;
(vii)
(SEQ ID NO: 36)
CAAAAACATCCCACGCCTAGT
and
(SEQ ID NO: 37)
TGGGGTGAAGACCATTTTGAA;
(viii)
(SEQ ID NO: 238)
GCTGCAGTGGGACCACTATTATC;
and
(SEQ ID NO: 239)
GCTCCCGGAAGACAGTCCCC;
(ix)
(SEQ ID NO: 240)
CTGGCTGGACCAAGCCCATCA;
and
(SEQ ID NO: 241)
CTAGGCGAGGAGCTCCAGTCG;
(x)
(SEQ ID NO: 38)
GAGAAGAGGGTTGAGGAGTTCAA
and
(SEQ ID NO: 39)
GCAAAATCACATTATTGCCAACAT;
(xi)
(SEQ ID NO: 236)
GGCACGGTCTTCAGAGAAGC;
and
(SEQ ID NO: 237)
ACATTATTGCCAACATGACTTACTTGAT;
(xii)
(SEQ ID NO: 40)
CTAGTGCATTCAAGCACAATGG
and
(SEQ ID NO: 41)
CAGAGCCTAAACATCCCCTTAAA;
(xii)
(SEQ ID NO: 42)
AGGTGATCTATTTTTCCCTTTCTCC
and
(SEQ ID NO: 43)
TTATGTGTACCCAAAAAGGTGACA;
(xiv)
(SEQ ID NO: 44)
TGCCAGTTGTGCTTTTTGCT
and
(SEQ ID NO: 45)
ACAATAAAAGGCAGCTTGGACAC;
(xv)
(SEQ ID NO: 46)
ACTTGGCAGCCAGAAATATCCTC
and
(SEQ ID NO: 47)
GACTGTCAAGCAGAGAATGGGTACT;
(xvi)
(SEQ ID NO: 232)
GTATTCACAGAGACTTGGCAGC;
and
(SEQ ID NO: 233)
TGTCAAGCAGAGAATGGGTACT;
(xvii)
(SEQ ID NO: 48)
GATGTCTATGAAGTGTTGTGGTTCC
and
(SEQ ID NO: 49)
TGTTACAGAAATGAAATAAGACGGAAA;
(xviii)
(SEQ ID NO: 50)
GAGGCTCGCCATGCCGGGAG
and
(SEQ ID NO: 51)
GATGTCGATGGACGTCTCGTGC;
(xix)
(SEQ ID NO: 52)
CAGCGCCGCCTTCGGCTTTC
and
(SEQ ID NO: 53)
GCCCGGGTAGTCAAAGTCGC;
(xx)
(SEQ ID NO: 54)
GGCAGCAGGAGAAGGCCAAG
and
(SEQ ID NO: 55)
TTCATCCTCCTCGCGGGGCTC;
(xxi)
(SEQ ID NO: 56)
CGGCCGCTGGTGATCAAGC
and
(SEQ ID NO: 57)
GGTGACCGGGCTGCAGGTG;
(xxii)
(SEQ ID NO: 58)
CGCACTGCGGCCAGACCAC
and
(SEQ ID NO: 59)
GTTGCTGTTCTTGTCCACCGAC;
(xxiii)
(SEQ ID NO: 60)
GCGGGCAAGGCCAAGAAGTC
and
(SEQ ID NO: 61)
GTGTCCAGTTCGCGGCTCAG;
(xxiv)
(SEQ ID NO: 62)
GACCAGTGACAATGACCGCCTG
and
(SEQ ID NO: 63)
GGCGACCCCAAACCACTCC;
(xxv) a pair of primers having the reverse complement sequences of any of (i) through (xxiv); and
(xxvi) a pair of primers having at least 90% sequence identity to any of (i) through (xxv);
(b) determining that the double-stranded oligonucleotide comprises or does not comprise the mutation in the gene of interest by means of a High Resolution Melting (HRM) analysis.

45. The method of claim 44, further comprising the step of sequencing the mutation.

46. The method of claim 44 or claim 45, wherein the sample is from a subject in need of treatment of an acute myeloid leukemia, and the method further comprises treating the subject with an effective amount of a therapeutic agent to treat the acute myeloid leukemia.

47. The method of any one of claims 44-46, wherein the determining step comprises comparing HRM analysis results of the double-stranded oligonucleotide to a control.

48. The method of claim 47, wherein the control comprises HRM analysis results of a wild-type sample.

49. The method of claim 47, wherein the control comprises HRM analysis results of a sample comprising the mutation of interest.

50. The method of any one of claims 47-49, wherein the HRM analysis is performed using a dye.

51. The method of claim 50, wherein the dye comprises SYTO® 9, SYBR® Green, or Chai Green™.

52. The method of any one of claims 44-51, wherein the amplifying step comprises performing PCR using, in parallel, at least 2 pairs of primers from (i) through (xxvi).

53. The method of any one of claims 44-51, wherein the amplifying step comprises performing PCR using, in parallel, at least 3 pairs of primers from (i) through (xxvi).

54. The method of any one of claims 44-51, wherein the amplifying step comprises performing PCR using, in parallel, at least 4 pairs of primers from (i) through (xxvi).

55. The method of any one of claims 44-54, wherein the gene of interest comprises FLT3, and wherein the pair of primers are selected from the list consisting of:
(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 32)
CTTGTCACCCACGGGAAAGT
and
(SEQ ID NO: 33)
AAAATAAGTAGGAAATAGCAGCCTCA;
(iv)
(SEQ ID NO: 234)
AGTTTTACATTTTTAATGCTCCTTTCTT
and
(SEQ ID NO: 235)
TGATGAGGTGATTTTCGTGGA;
(v) a pair of primers having the reverse complement sequences of any of (i) through (iv); and
(vi) a pair of primers having at least 90% sequence identity to any of (i) through (v).

56. The method of any one of claims 44-54, wherein the gene of interest comprises IDH2, and wherein the pair of primers are selected from the list consisting of:
(i) (vi)
(SEQ ID NO: 34)
TCTCTGTCCTCACAGAGTTCAAGC
and
(SEQ ID NO: 35)
CTTGGTCCAGCCAGGGACTA;
(ii)
(SEQ ID NO: 36)
CAAAAACATCCCACGCCTAGT
and
(SEQ ID NO: 37)
TGGGGTGAAGACCATTTTGAA;
(iii)
(SEQ ID NO: 238)
GCTGCAGTGGGACCACTATTATC
and
(SEQ ID NO: 239)
GCTCCCGGAAGACAGTCCCC;
(iv)
(SEQ ID NO: 240)
CTGGCTGGACCAAGCCCATCA
and
(SEQ ID NO: 241)
CTAGGCGAGGAGCTCCAGTCG;
(v) a pair of primers having the reverse complement sequences of any of (i) through (iv); and
(vi) a pair of primers having at least 90% sequence identity to any of (i) through (v).

57. The method of any one of claims 44-54, wherein the gene of interest comprises IDH1, and wherein the pair of primers are selected from the list consisting of:
(i)
(SEQ ID NO: 38)
GAGAAGAGGGTTGAGGAGTTCAA
and
(SEQ ID NO: 39)
GCAAAATCACATTATTGCCAACAT;
(ii)
(SEQ ID NO: 236)
GGCACGGTCTTCAGAGAAGC
and
(SEQ ID NO: 237)
ACATTATTGCCAACATGACTTACTTGAT;
(iii) a pair of primers having the reverse complement sequences of any of (i) through (ii); and
(iv) a pair of primers having at least 90% sequence identity to any of (i) through (iii).

58. The method of any one of claims 44-54, wherein the gene of interest comprises KIT, and wherein the pair of primers are selected from the list consisting of:
(i)
(SEQ ID NO: 40)
CTAGTGCATTCAAGCACAATGG
and
(SEQ ID NO: 41)
CAGAGCCTAAACATCCCCTTAAA;
(ii)
(SEQ ID NO: 42)
AGGTGATCTATTTTTCCCTTTCTCC
and
(SEQ ID NO: 43)
TTATGTGTACCCAAAAAGGTGACA;
(iii)
(SEQ ID NO: 44)
TGCCAGTTGTGCTTTTTGCT
and
(SEQ ID NO: 45)
ACAATAAAAGGCAGCTTGGACAC;
(v)
(SEQ ID NO: 232)
GTATTCACAGAGACTTGGCAGC
and
(SEQ ID NO: 233)
TGTCAAGCAGAGAATGGGTACT;
(vi) a pair of primers having the reverse complement sequences of any of (i) through (v); and
(vii) a pair of primers having at least 90% sequence identity to any of (i) through (vi).

59. The method of any one of claims 44-54, wherein the gene of interest comprises NMP1, and wherein the pair of primers are selected from the list consisting of:
(i)
(SEQ ID NO: 48)
GATGTCTATGAAGTGTTGTGGTTCC
and
(SEQ ID NO: 49)
TGTTACAGAAATGAAATAAGACGGAAA;
(ii) a pair of primers having the reverse complement sequences of (i); and
(iii) a pair of primers having at least 90% sequence identity to any of (i) through (ii).

60. The method of any one of claims 44-54, wherein the gene of interest comprises CEBPA, and wherein the pair of primers are selected from the list consisting of:
(i)
(SEQ ID NO: 50)
GAGGCTCGCCATGCCGGGAG
and
(SEQ ID NO: 51)
GATGTCGATGGACGTCTCGTGC;
(ii)
(SEQ ID NO: 52)
CAGCGCCGCCTTCGGCTTTC
and
(SEQ ID NO: 53)
GCCCGGGTAGTCAAAGTCGC;
(iii)
(SEQ ID NO: 54)
GGCAGCAGGAGAAGGCCAAG
and
(SEQ ID NO: 55)
TTCATCCTCCTCGCGGGGCTC;
(iv)
(SEQ ID NO: 56)
CGGCCGCTGGTGATCAAGC
and
(SEQ ID NO: 57)
GGTGACCGGGCTGCAGGTG;
(v)
(SEQ ID NO: 58)
CGCACTGCGGCCAGACCAC
and
(SEQ ID NO: 59)
GTTGCTGTTCTTGTCCACCGAC;
(vi)
(SEQ ID NO: 60)
GCGGGCAAGGCCAAGAAGTC
and
(SEQ ID NO: 61)
GTGTCCAGTTCGCGGCTCAG;
(vii)
(SEQ ID NO: 62)
GACCAGTGACAATGACCGCCTG
and
(SEQ ID NO: 63)
GGCGACCCCAAACCACTCC;
(viii) a pair of primers having the reverse complement sequences of any of (i) through (vii); and
(ix) a pair of primers having at least 90% sequence identity to any of (i) through (viii).

61. The method of any one of claims 44-60, wherein at least one of the pair of primers comprises a tag.

62. The method of claim 61, wherein the tag comprises M13F.

63. A method of treating a subject in need of treatment of acute myeloid leukemia, the method comprising:
(a) amplifying a region of a gene of interest from a sample from the subject to produce a double-stranded oligonucleotide, wherein the amplifying step comprises performing PCR using a pair of primers, selected from the group consisting of:
(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 30)
TTACATTTTTAATGCTCCTTTCTTTGA
and
(SEQ ID NO: 31)
GATGAGGTGATTTTCGTGGAAG;
(iv)
(SEQ ID NO: 32)
CTTGTCACCCACGGGAAAGT
and
(SEQ ID NO: 33)
AAAATAAGTAGGAAATAGCAGCCTCA;
(v)
(SEQ ID NO: 234)
AGTTTTACATTTTTAATGCTCCTTTCTT
and
(SEQ ID NO: 235)
TGATGAGGTGATTTTCGTGGA;
(vi)
(SEQ ID NO: 34)
TCTCTGTCCTCACAGAGTTCAAGC
and
(SEQ ID NO: 35)
CTTGGTCCAGCCAGGGACTA;
(vii)
(SEQ ID NO: 36)
CAAAAACATCCCACGCCTAGT
and
(SEQ ID NO: 37)
TGGGGTGAAGACCATTTTGAA;
(viii)
(SEQ ID NO: 238)
GCTGCAGTGGGACCACTATTATC
and
(SEQ ID NO: 239)
GCTCCCGGAAGACAGTCCCC;
(ix)
(SEQ ID NO: 240)
CTGGCTGGACCAAGCCCATCA
and
(SEQ ID NO: 241)
CTAGGCGAGGAGCTCCAGTCG;
(x)
(SEQ ID NO: 38)
GAGAAGAGGGTTGAGGAGTTCAA
and
(SEQ ID NO: 39)
GCAAAATCACATTATTGCCAACAT;
(xi)
(SEQ ID NO: 236)
GGCACGGTCTTCAGAGAAGC
and
(SEQ ID NO: 237)
ACATTATTGCCAACATGACTTACTTGAT;
(xii)
(SEQ ID NO: 40)
CTAGTGCATTCAAGCACAATGG
and
(SEQ ID NO: 41)
CAGAGCCTAAACATCCCCTTAAA;
(xiii)
(SEQ ID NO: 42)
AGGTGATCTATTTTTCCCTTTCTCC
and
(SEQ ID NO: 43)
TTATGTGTACCCAAAAAGGTGACA;
(xiv)
(SEQ ID NO: 44)
TGCCAGTTGTGCTTTTTGCT
and
(SEQ ID NO: 45)
ACAATAAAAGGCAGCTTGGACAC;
(XV)
(SEQ ID NO: 46)
ACTTGGCAGCCAGAAATATCCTC
and
(SEQ ID NO: 47)
GACTGTCAAGCAGAGAATGGGTACT;
(xvi)
(SEQ ID NO: 232)
GTATTCACAGAGACTTGGCAGC
and
(SEQ ID NO: 233)
TGTCAAGCAGAGAATGGGTACT;
(xvii)
(SEQ ID NO: 48)
GATGTCTATGAAGTGTTGTGGTTCC
and
(SEQ ID NO: 49)
TGTTACAGAAATGAAATAAGACGGAAA;
(xviii)
(SEQ ID NO: 50)
GAGGCTCGCCATGCCGGGAG
and
(SEQ ID NO: 51)
GATGTCGATGGACGTCTCGTGC;
(xix)
(SEQ ID NO: 52)
CAGCGCCGCCTTCGGCTTTC
and
(SEQ ID NO: 53)
GCCCGGGTAGTCAAAGTCGC;
(xx)
(SEQ ID NO: 54)
GGCAGCAGGAGAAGGCCAAG
and
(SEQ ID NO: 55)
TTCATCCTCCTCGCGGGGCTC;
(xxi)
(SEQ ID NO: 56)
CGGCCGCTGGTGATCAAGC
and
(SEQ ID NO: 57)
GGTGACCGGGCTGCAGGTG;
(xxii)
(SEQ ID NO: 58)
CGCACTGCGGCCAGACCAC
and
(SEQ ID NO: 59)
GTTGCTGTTCTTGTCCACCGAC;
(xxiii)
(SEQ ID NO: 60)
GCGGGCAAGGCCAAGAAGTC
and
(SEQ ID NO: 61)
GTGTCCAGTTCGCGGCTCAG;
(xxiv)
(SEQ ID NO: 62)
GACCAGTGACAATGACCGCCTG
and
(SEQ ID NO: 63)
GGCGACCCCAAACCACTCC;
(xxv) a pair of primers having the reverse complement sequences of any of (i) through (xxiv); and
(xxvi) a pair of primers having at least 90% sequence identity to any of (i) through (xxv);
(b) determining that the double-stranded oligonucleotide comprises a mutation in the gene of interest by means of a High Resolution Melting (HRM) analysis;
(c) sequencing the mutation;
(d) treating the subject in need of treatment of the acute myeloid leukemia with an effective amount of a therapeutic agent.

64. The method of claim 63, wherein the determining step comprises comparing HRM analysis results of the double-stranded oligonucleotide to a control.

65. The method of claim 64, wherein the control comprises HRM analysis results of a wild-type sample.

66. The method of claim 65, wherein the control comprises HRM analysis results of a sample comprising the mutation of interest.

67. The method of any of claims 63-66, wherein the HRM analysis is performed using a dye.

68. The method of claim 67, wherein the dye comprises SYTO® 9, SYBR® Green, or Chai Green™.

69. The method of any one of claims 63-68, wherein the amplifying step comprises performing PCR using, in parallel, at least 2 pairs of primers from (i) through (xxvi).

70. The method of any one of claims 63-68, wherein the amplifying step comprises performing PCR using, in parallel, at least 3 pairs of primers from (i) through (xxvi).

71. The method of any one of claims 63-68, wherein the amplifying step comprises performing PCR using, in parallel, at least 4 pairs of primers from (i) through (xxvi).

72. The method of any preceding claim, wherein the gene of interest comprises FLT3, and wherein the pair of primers are selected from the list consisting of:
(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(iii)
(SEQ ID NO: 32)
CTTGTCACCCACGGGAAAGT
and
(SEQ ID NO: 33)
AAAATAAGTAGGAAATAGCAGCCTCA;
(iv)
(SEQ ID NO: 234)
AGTTTTACATTTTTAATGCTCCTTTCTT
and
(SEQ ID NO: 235)
TGATGAGGTGATTTTCGTGGA;
(v) a pair of primers having the reverse complement sequences of any of (i) through (iv); and
(vi) a pair of primers having at least 90% sequence identity to any of (i) through (v).

73. The method of any one of claims 63-73, wherein the gene of interest comprises IDH2, and wherein the pair of primers are selected from the list consisting of:
(i) (vi)
(SEQ ID NO: 34)
TCTCTGTCCTCACAGAGTTCAAGC
and
(SEQ ID NO: 35)
CTTGGTCCAGCCAGGGACTA;
(ii)
(SEQ ID NO: 36)
CAAAAACATCCCACGCCTAGT
and
(SEQ ID NO: 37)
TGGGGTGAAGACCATTTTGAA;
(iii)
(SEQ ID NO: 238)
GCTGCAGTGGGACCACTATTATC
and
(SEQ ID NO: 239)
GCTCCCGGAAGACAGTCCCC;
(iv)
(SEQ ID NO: 240)
CTGGCTGGACCAAGCCCATCA
and
(SEQ ID NO: 241)
CTAGGCGAGGAGCTCCAGTCG;
(v) a pair of primers having the reverse complement sequences of any of (i) through (iv); and
(vi) a pair of primers having at least 90% sequence identity to any of (i) through (v).

74. The method of any one of claims 63-73, wherein the gene of interest comprises IDH1, and wherein the pair of primers are selected from the list consisting of:
(i)
(SEQ ID NO: 38)
GAGAAGAGGGTTGAGGAGTTCAA
and
(SEQ ID NO: 39)
GCAAAATCACATTATTGCCAACAT;
(ii)
(SEQ ID NO: 236)
GGCACGGTCTTCAGAGAAGC
and
(SEQ ID NO: 237)
ACATTATTGCCAACATGACTTACTTGAT;
(iii) a pair of primers having the reverse complement sequences of any of (i) through (ii); and
(iv) a pair of primers having at least 90% sequence identity to any of (i) through (iii).

75. The method of any one of claims 63-73, wherein the gene of interest comprises KIT, and wherein the pair of primers are selected from the list consisting of:
(i)
(SEQ ID NO: 40)
CTAGTGCATTCAAGCACAATGG
and
(SEQ ID NO: 41)
CAGAGCCTAAACATCCCCTTAAA;
(ii)
(SEQ ID NO: 42)
AGGTGATCTATTTTTCCCTTTCTCC
and
(SEQ ID NO: 43)
TTATGTGTACCCAAAAAGGTGACA;
(iii)
(SEQ ID NO: 44)
TGCCAGTTGTGCTTTTTGCT
and
(SEQ ID NO: 45)
ACAATAAAAGGCAGCTTGGACAC;
(v)
(SEQ ID NO: 232)
GTATTCACAGAGACTTGGCAGC
and
(SEQ ID NO: 233)
TGTCAAGCAGAGAATGGGTACT;
(vi) a pair of primers having the reverse complement sequences of any of (i) through (v); and
(vii) a pair of primers having at least 90% sequence identity to any of (i) through (vi).

76. The method of any one of claims 63-73, wherein the gene of interest comprises NMP1, and wherein the pair of primers are selected from the list consisting of:
(i)
(SEQ ID NO: 48)
GATGTCTATGAAGTGTTGTGGTTCC
and
(SEQ ID NO: 49)
TGTTACAGAAATGAAATAAGACGGAAA;
(ii) a pair of primers having the reverse complement sequences of (i); and
(iii) a pair of primers having at least 90% sequence identity to any of (i) through (ii).

77. The method of any one of claims 63-73, wherein the gene of interest comprises CEBPA, and wherein the pair of primers are selected from the list consisting of:
(i)
(SEQ ID NO: 50)
GAGGCTCGCCATGCCGGGAG
and
(SEQ ID NO: 51)
GATGTCGATGGACGTCTCGTGC;
(ii)
(SEQ ID NO: 52)
CAGCGCCGCCTTCGGCTTTC
and
(SEQ ID NO: 53)
GCCCGGGTAGTCAAAGTCGC;
(iii)
(SEQ ID NO: 54)
GGCAGCAGGAGAAGGCCAAG
and
(SEQ ID NO: 55)
TTCATCCTCCTCGCGGGGCTC;
(iv)
(SEQ ID NO: 56)
CGGCCGCTGGTGATCAAGC
and
(SEQ ID NO: 57)
GGTGACCGGGCTGCAGGTG;
(v)
(SEQ ID NO: 58)
CGCACTGCGGCCAGACCAC
and
(SEQ ID NO: 59)
GTTGCTGTTCTTGTCCACCGAC;
(vi)
(SEQ ID NO: 60)
GCGGGCAAGGCCAAGAAGTC
and
(SEQ ID NO: 61)
GTGTCCAGTTCGCGGCTCAG;
(vii)
(SEQ ID NO: 62)
GACCAGTGACAATGACCGCCTG
and
(SEQ ID NO: 63)
GGCGACCCCAAACCACTCC;
(vii) a pair of primers having the reverse complement sequences of any of (i) through (vii); and
(ix) a pair of primers having at least 90% sequence identity to any of (i) through (viii).

78. The method of any one of claims 63-77, wherein at least one of the pair of primers comprises a tag.

79. The method of claim 78, wherein the tag comprises M13F.

80. A kit comprising:
(a) a pair of primers capable of amplifying a region of a gene of interest from a sample to produce a double-stranded oligonucleotide, wherein the pair of primers is selected from the group consisting of:
(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTIGTC
and
(SEQ ID NO: 29)
CATCTTTGTTGCTGTCCTTCCA;
(iii)
(SEQ ID NO: 30)
TTACATTTTTAATGCTCCTTTCTTTGA
and
(SEQ ID NO: 31)
GATGAGGTGATTTTCGTGGAAG;
(iv)
(SEQ ID NO: 32)
CTTGTCACCCACGGGAAAGT
and
(SEQ ID NO: 33)
AAAATAAGTAGGAAATAGCAGCCTCA;
(v)
(SEQ ID NO: 234)
AGTTTTACATTTTTAATGCTCCTTTCTT
and
(SEQ ID NO: 235)
TGATGAGGTGATTTTCGTGGA;
(vi)
(SEQ ID NO: 34)
TCTCTGTCCTCACAGAGTTCAAGC
and
(SEQ ID NO: 35)
CTTGGTCCAGCCAGGGACTA;
(vii)
(SEQ ID NO: 36)
CAAAAACATCCCACGCCTAGT
and
(SEQ ID NO: 37)
TGGGGTGAAGACCATTTTGAA;
(viii)
(SEQ ID NO: 238)
GCTGCAGTGGGACCACTATTATC
and
(SEQ ID NO: 239)
GCTCCCGGAAGACAGTCCCC;
(ix)
(SEQ ID NO: 240)
CTGGCTGGACCAAGCCCATCA
and
(SEQ ID NO: 241)
CTAGGCGAGGAGCTCCAGTCG;
(x)
(SEQ ID NO: 38)
GAGAAGAGGGTTGAGGAGTTCAA
and
(SEQ ID NO: 39)
GCAAAATCACATTATTGCCAACAT;
(xi)
(SEQ ID NO: 236)
GGCACGGTCTTCAGAGAAGC
and
(SEQ ID NO: 237)
ACATTATTGCCAACATGACTTACTTGAT;
(xii)
(SEQ ID NO: 40)
CTAGTGCATTCAAGCACAATGG
and
(SEQ ID NO: 41)
CAGAGCCTAAACATCCCCTTAAA;
(xiii)
(SEQ ID NO: 42)
AGGTGATCTATTTTTCCCTTTCTCC
and
(SEQ ID NO: 43)
TTATGTGTACCCAAAAAGGTGACA;
(xiv)
(SEQ ID NO: 44)
TGCCAGTTGTGCTTTTTGCT
and
(SEQ ID NO: 45)
ACAATAAAAGGCAGCTTGGACAC;
(XV)
(SEQ ID NO: 46)
ACTTGGCAGCCAGAAATATCCTC
and
(SEQ ID NO: 47)
GACTGTCAAGCAGAGAATGGGTACT;
(xvi)
(SEQ ID NO: 232)
GTATTCACAGAGACTTGGCAGC
and
(SEQ ID NO: 233)
TGTCAAGCAGAGAATGGGTACT;
(xvii)
(SEQ ID NO: 48)
GATGTCTATGAAGTGTTGTGGTTCC
and
(SEQ ID NO: 49)
TGTTACAGAAATGAAATAAGACGGAAA;
(xviii)
(SEQ ID NO: 50)
GAGGCTCGCCATGCCGGGAG
and
(SEQ ID NO: 51)
GATGTCGATGGACGTCTCGTGC;
(xix)
(SEQ ID NO: 52)
CAGCGCCGCCTTCGGCTTTC
and
(SEQ ID NO: 53)
GCCCGGGTAGTCAAAGTCGC;
(xx)
(SEQ ID NO: 54)
GGCAGCAGGAGAAGGCCAAG
and
(SEQ ID NO: 55)
TTCATCCTCCTCGCGGGGCTC;
(xxi)
(SEQ ID NO: 56)
CGGCCGCTGGTGATCAAGC
and
(SEQ ID NO: 57)
GGTGACCGGGCTGCAGGTG;
(xxii)
(SEQ ID NO: 58)
CGCACTGCGGCCAGACCAC
and
(SEQ ID NO: 59)
GTTGCTGTTCTTGTCCACCGAC;
(xxiii)
(SEQ ID NO: 60)
GCGGGCAAGGCCAAGAAGTC
and
(SEQ ID NO: 61)
GTGTCCAGTTCGCGGCTCAG;
(xxiv)
(SEQ ID NO: 62)
GACCAGTGACAATGACCGCCTG
and
(SEQ ID NO: 63)
GGCGACCCCAAACCACTCC;
(xxv) a pair of primers having the reverse complement sequences of any of (i) through (xxiv); and
(xxvi) a pair of primers having at least 90% sequence identity to any of (i) through (xxv);
(b) one or more reagents suitable for performing High Resolution Melting (HRM) analysis.

81. The kit of claim 80, wherein the kit comprises at least 2 pairs of primers from (i) through (xxvi).

82. The kit of claim 80, wherein the kit comprises at least 3 pairs of primers from (i) through (xxvi).

83. The kit of claim 80, wherein the kit comprises at least 4 pairs of primers from (i) through (xxvi).

84. The kit of any of claims 80-83, further comprising a wild-type control.

85. The kit of any of claims 80-84, further comprising a mutant control.

86. The kit of any of claims 80-85, further comprising a dye.

87. The kit of claim 86, wherein the dye comprises SYTO® 9, SYBR® Green, or Chai Green™.

88. The kit of any one of claims 80-87, wherein the gene of interest comprises FLT3, and wherein the pair of primers are selected from the list consisting of:
(i)
(SEQ ID NO: 26)
TGCCTATTCCTAACTGACTCATCATT
and
(SEQ ID NO: 27)
GAAACATTTGGCACATTCCATTC;
(ii)
(SEQ ID NO: 28)
AATGCACGTACTCACCATTTGTC
and
(SEQ ID NO: 29)
CATCTTTGTTGCTGTCCTTCCA;
(SEQ ID NO: 32)
CTTGTCACCCACGGGAAAGT
and
(SEQ ID NO: 33)
AAAATAAGTAGGAAATAGCAGCCTCA;
(iv)
(SEQ ID NO: 234)
AGTTTTACATTTTTAATGCTCCTTTCTT
and
(SEQ ID NO: 235)
TGATGAGGTGATTTTCGTGGA;
(v) a pair of primers having the reverse complement sequences of any of (i) through (iv); and
(vi) a pair of primers having at least 90% sequence identity to any of (i) through (v).

89. The kit of any one of claims 80-87, wherein the gene of interest comprises IDH2, and wherein the pair of primers are selected from the list consisting of:
(i)
(vi)
(SEQ ID NO: 34)
TCTCTGTCCTCACAGAGTTCAAGC
and
(SEQ ID NO: 35)
CTTGGTCCAGCCAGGGACTA;
(ii)
(SEQ ID NO: 36)
CAAAAACATCCCACGCCTAGT
and
(SEQ ID NO: 37)
TGGGGTGAAGACCATTTTGAA;
(iii)
(SEQ ID NO: 238)
GCTGCAGTGGGACCACTATTATC
and
(SEQ ID NO: 239)
GCTCCCGGAAGACAGTCCCC;
(iv)
(SEQ ID NO: 240)
CTGGCTGGACCAAGCCCATCA
and
(SEQ ID NO: 241)
CTAGGCGAGGAGCTCCAGTCG;
(v) a pair of primers having the reverse complement sequences of any of (i) through (iv); and
(vi) a pair of primers having at least 90% sequence identity to any of (i) through (v).

90. The kit of any one of claims 80-87, wherein the gene of interest comprises IDH1, and wherein the pair of primers are selected from the list consisting of:
(i)
(SEQ ID NO: 38)
GAGAAGAGGGTTGAGGAGTTCAA;
and
(SEQ ID NO: 39)
GCAAAATCACATTATTGCCAACAT;
(ii)
(SEQ ID NO: 236)
GGCACGGTCTTCAGAGAAGC;
and
(SEQ ID NO: 237)
ACATTATTGCCAACATGACTTACTTGAT;
(iii) a pair of primers having the reverse complement sequences of any of (i) through (ii); and
(iv) a pair of primers having at least 90% sequence identity to any of (i) through (iii).

91. The kit of any one of claims 80-87, wherein the gene of interest comprises KIT, and wherein the pair of primers are selected from the list consisting of:
(i)
(SEQ ID NO: 40)
CTAGTGCATTCAAGCACAATGG;
and
(SEQ ID NO: 41)
CAGAGCCTAAACATCCCCTTAAA;
(ii)
(SEQ ID NO: 42)
AGGTGATCTATTTTTCCCTTTCTCC;
and
(SEQ ID NO: 43)
TTATGTGTACCCAAAAAGGTGACA;
iii)
(SEQ ID NO: 44)
TGCCAGTTGTGCTTTTTGCT;
and
(SEQ ID NO: 45)
ACAATAAAAGGCAGCTTGGACAC;
(v)
(SEQ ID NO: 232)
GTATTCACAGAGACTTGGCAGC;
and
(SEQ ID NO: 233)
TGTCAAGCAGAGAATGGGTACT;
(vi) a pair of primers having the reverse complement sequences of any of (i) through (v); and
(vii) a pair of primers having at least 90% sequence identity to any of (i) through (vi).

92. The kit of any one of claims 80-87, wherein the gene of interest comprises NMP1, and wherein the pair of primers are selected from the list consisting of:
(i)
(SEQ ID NO: 48)
GATGTCTATGAAGTGTTGTGGTTCC;
and
(SEQ ID NO: 49)
TGTTACAGAAATGAAATAAGACGGAAA;
(ii) a pair of primers having the reverse complement sequences of (i); and
(iii) a pair of primers having at least 90% sequence identity to any of (i) through (ii).

93. The kit of any one of claims 80-87, wherein the gene of interest comprises CEBPA, and wherein the pair of primers are selected from the list consisting of:
(i)
(SEQ ID NO: 50)
GAGGCTCGCCATGCCGGGAG;
and
(SEQ ID NO: 51)
GATGTCGATGGACGTCTCGTGC;
(ii)
(SEQ ID NO: 52)
CAGCGCCGCCTTCGGCTTTC;
and
(SEQ ID NO: 53)
GCCCGGGTAGTCAAAGTCGC;
(iii)
(SEQ ID NO: 54)
GGCAGCAGGAGAAGGCCAAG;
and
(SEQ ID NO: 55)
TTCATCCTCCTCGCGGGGCTC;
(iv)
(SEQ ID NO: 56)
CGGCCGCTGGTGATCAAGC;
and
(SEQ ID NO: 57)
GGTGACCGGGCTGCAGGTG;
(v)
(SEQ ID NO: 58)
CGCACTGCGGCCAGACCAC;
and
(SEQ ID NO: 59)
GTTGCTGTTCTTGTCCACCGAC;
(vi)
(SEQ ID NO: 60)
GCGGGCAAGGCCAAGAAGTC;
and
(SEQ ID NO: 61)
GTGTCCAGTTCGCGGCTCAG;
(vii)
(SEQ ID NO: 62)
GACCAGTGACAATGACCGCCTG;
and
(SEQ ID NO: 63)
GGCGACCCCAAACCACTCC;
(vii) a pair of primers having the reverse complement sequences of any of (i) through (vii); and
(ix) a pair of primers having at least 90% sequence identity to any of (i) through (viii).

94. The kit of any one of claims 80-93, wherein at least one of the pair of primers comprises a tag.

95. The kit of claim 94, wherein the tag comprises M13F.

96. A method for determining the presence or absence of a mutation in a gene of interest, the method comprising:
(a) amplifying a region of a gene of interest from a sample to produce a double-stranded oligonucleotide, wherein the amplifying step comprises performing PCR using a pair of primers, selected from the group consisting of:
(i)
(SEQ ID NO: 64)
CCTTCCTCTTACTCTCTGCCTGC
and
(SEQ ID NO: 65)
GACAGCGGGCACACTTACCAG;
(ii)
(SEQ ID NO: 66)
CATTGTTAGGGCCGAGGCTAGA
and
(SEQ ID NO: 67)
ACGCACTTGTTTTACCTGTATGAGTC;
(iii)
(SEQ ID NO: 218)
GCCTCCCTTCCTCTTACTCTCT
and
GCAGCCTGGGTAAGCACAC;
(iv)
(SEQ ID NO: 68)
GCAGATTCAACTTTCACGTATCAAACC
and
(SEQ ID NO: 69)
TGCAGAGCACGGGCTTTAATGT;
(v)
(SEQ ID NO: 70)
CCTCGCAGACATTAAAGCCCGT
and
(SEQ ID NO: 71)
CCACCATCACCACTGCTGCTG;
(vi)
(SEQ ID NO: 72)
CAGTGGTGATGGTGGTGAGGC
and
(SEQ ID NO: 73)
CCTAGCCCATCTGTGAGTCCAAC;
(vii)
(SEQ ID NO: 74)
TGTCCTCCCAAACCTCAGTAGC
and
(SEQ ID NO: 75)
AGCTTGGCCAGTTCCTTTCTCT;
(viii)
(SEQ ID NO: 76)
GTGTCTCGAGTATGTGCGGTCC
and
(SEQ ID NO: 77)
CATCGTGGGCTGGTGGAAGAA;
(ix)
(SEQ ID NO: 192)
CTTTCACGTATCAAACCACCC
and
(SEQ ID NO: 193)
CACGGGCTTTAATGTCTGC;
(x)
(SEQ ID NO: 194)
AAGATCCCAGATTCCCTACTG
and
(SEQ ID NO: 195)
GTGGGCTGGTGGAAGAAC;
(xi)
(SEQ ID NO: 196)
GGCGAGAGGTCACCACTG
and
(SEQ ID NO: 197)
CCATCACCACTGCTGCTG;
(xii)
(SEQ ID NO: 198)
AACTGAATGTGAGTCTGGCA
and
(SEQ ID NO: 199)
CACTAGAGACGGAATGGGAC;
(xiii)
(SEQ ID NO: 200)
CAGTGGTGATGGTGGTGAG
and
(SEQ ID NO: 201)
CCAACTGTAGCCCTCTGTAG;
(xiv)
(SEQ ID NO: 78)
TCCATTGCCTCTCCTTCTGTGC
and
(SEQ ID NO: 79)
TGCCAACTCCTTCATGCACCT;
(xv)
(SEQ ID NO: 80)
CCTAGGGGATGTTCCAGATGGC
and
(SEQ ID NO: 81)
AATGTGGGTTTGTTGCCATGAAAC;
(xvi)
(SEQ ID NO: 82)
TCTATCGTGTCCCCACAGGGAA
and
(SEQ ID NO: 83)
GGGGAAAGGTTGAACCCAAGGA;
(xvii)
(SEQ ID NO: 84)
CTTCACGCCGCCTTCCACCG
and
(SEQ ID NO: 85)
CTCCGGGCCAGTACCTTGAAA;
(xviii)
(SEQ ID NO: 204)
AGCAACGCCCATTTCACC
and
(SEQ ID NO: 205)
GCTCAGCTGCAAAGAATGTG;
(xix)
(SEQ ID NO: 206)
CAGGCAAGATGAGCGAGGC
and
(SEQ ID NO: 207)
GGGCCAGTACCTTGAAAGCG;
(xx)
(SEQ ID NO: 86)
TCCTGCTGTGTGGTTAGACGG
and
(SEQ ID NO: 87)
GAAGAGGTGGCGGATGACTGG;
(xxi)
(SEQ ID NO: 202)
CTGCTGTGTGGTTAGACGG
and
(SEQ ID NO: 203)
GAGGTGGCGGATGACTGG;
(xxii)
(SEQ ID NO: 88)
GCCCTGGGCATTCCTTCTTTATT
and
(SEQ ID NO: 89)
ACTTCTAAGATGTGGCAAGATGGC;
(xxiii)
(SEQ ID NO: 90)
GTTGGGGCATAGTTAAAACCTGTGT
and
(SEQ ID NO: 91)
TTCCTCTGTGTTGGCGGATACC;
(xxiv)
(SEQ ID NO: 92)
GGTATCCGCCAACACAGAGGAA
and
(SEQ ID NO: 93)
AGGAGACTGGAATTCTCGAATAAGGA;
(XXV)
(SEQ ID NO: 208)
GGCTGCTGGTCTGGCTACTAT
and
(SEQ ID NO: 209)
CCAGGGCAGAGGCTACAAC;
(xxVi)
(SEQ ID NO: 210)
TGTAGGTCTTGTGGATGAGC
and
(SEQ ID NO: 211)
TTCCTCTGTGTTGGCGGATA;
(xxvii)
(SEQ ID NO: 212)
CAGAGGAAAGGTAAATCCACCA
and
(SEQ ID NO: 213)
GGAATAAGATACCCAATAGCCTTCA;
(xxviii)
(SEQ ID NO: 214)
TTCCTTCTTTATTGCCCTTCTTA
and
(SEQ ID NO: 215)
CAACTTACCATGTTCAATGATTTCAA;
(xxix) a pair of primers having the reverse complement sequences of any of (i) through (xxviii); and
(xxx) a pair of primers having at least 90% sequence identity to any of (i) through (xxix);
(b) determining that the double-stranded oligonucleotide comprises or does not comprise the mutation in the gene of interest by means of a High Resolution Melting (HRM) analysis.

97. The method of claim 96, further comprising the step of sequencing the mutation.

98. The method of claim 96 or claim 97, wherein the sample is from a subject in need of treatment of anemia (ANM) or cytopenia, and the method further comprises treating the subject with an effective amount of a therapeutic agent to treat the anemia or cytopenia.

99. The method of any one of claims 96-98, wherein the determining step comprises comparing HRM analysis results of the double-stranded oligonucleotide to a control.

100. The method of claim 99, wherein the control comprises HRM analysis results of a wild-type sample.

101. The method of claim 99, wherein the control comprises HRM analysis results of a sample comprising the mutation of interest.

102. The method of any one of claims 99-101, wherein the HRM analysis is performed using a dye.

103. The method of claim 102, wherein the dye comprises SYTO® 9, SYBR® Green, or Chai Green™.

104. The method of any one of claims 96-103, wherein the amplifying step comprises performing PCR using, in parallel, at least 2 pairs of primers from (i) through (xxx).

105. The method of any one of claims 96-104, wherein the amplifying step comprises performing PCR using, in parallel, at least 3 pairs of primers from (i) through (xxx).

106. The method of any one of claims 96-105, wherein the amplifying step comprises performing PCR using, in parallel, at least 4 pairs of primers from (i) through (xxx).

107. The method of any one of claims 96-106, wherein the gene of interest comprises WT1, and wherein the pair of primers are selected from the list consisting of:
(i)
(SEQ ID NO: 218)
GCCTCCCTTCCTCTTACTCTCT;
and
GCAGCCTGGGTAAGCACAC;
(ii) a pair of primers having the reverse complement sequences of (i); and
(iii) a pair of primers having at least 90% sequence identity to any of (i) through (ii).

108. The method of any one of claims 96-106, wherein the gene of interest comprises ASXL1, and wherein the pair of primers are selected from the list consisting of:
(i)
(SEQ ID NO: 192)
CTTTCACGTATCAAACCACCC
and
(SEQ ID NO: 193)
CACGGGCTTTAATGTCTGC;
(ii)
(SEQ ID NO: 194)
AAGATCCCAGATTCCCTACTG
and
(SEQ ID NO: 195)
GTGGGCTGGTGGAAGAAC;
(iii)
(SEQ ID NO: 196)
GGCGAGAGGTCACCACTG
and
(SEQ ID NO: 197)
CCATCACCACTGCTGCTG;
(iv)
(SEQ ID NO: 198)
AACTGAATGTGAGTCTGGCA
and
(SEQ ID NO: 199)
CACTAGAGACGGAATGGGAC;
(v)
(SEQ ID NO: 200)
CAGTGGTGATGGTGGTGAG
and
(SEQ ID NO: 201)
CCAACTGTAGCCCTCTGTAG;
(vi) a pair of primers having the reverse complement sequences of any of (i) through (v); and
(vii) a pair of primers having at least 90% sequence identity to any of (i) through (vi).

109. The method of any one of claims 96-106, wherein the gene of interest comprises RUNX1, and wherein the pair of primers are selected from the list consisting of:
(i)
(SEQ ID NO: 204)
AGCAACGCCCATTTCACC;
and
(SEQ ID NO: 205)
GCTCAGCTGCAAAGAATGTG;
(ii)
(SEQ ID NO: 206)
CAGGCAAGATGAGCGAGGC;
and
(SEQ ID NO: 207)
GGGCCAGTACCTTGAAAGCG;
(iii) a pair of primers having the reverse complement sequences of any of (i) through (ii); and
(iv) a pair of primers having at least 90% sequence identity to any of (i) through (iii).

110. The method of any one of claims 96-106, wherein the gene of interest comprises DNMT3A, and wherein the pair of primers are selected from the list consisting of:
(i)
(SEQ ID NO: 202)
CTGCTGTGTGGTTAGACGG;
and
(SEQ ID NO: 203)
GAGGTGGCGGATGACTGG;
(ii) a pair of primers having the reverse complement sequences of (i); and
(iii) a pair of primers having at least 90% sequence identity to any of (i) through (ii).

111. The method of any one of claims 96-106, wherein the gene of interest comprises SFB31, and wherein the pair of primers are selected from the list consisting of:
(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;
(v) a pair of primers having the reverse complement sequences of any of (i) through (iv); and
(vi) a pair of primers having at least 90% sequence identity to any of (i) through (v).

112. The method of any one of claims 96-111, wherein at least one of the pair of primers comprises a tag.

113. The method of claim 112, wherein the tag comprises M13F.

114. A method of treating a subject in need of treatment of anemia or cytopenia, the method comprising:
(a) amplifying a region of a gene of interest from a sample from the subject to produce a double-stranded oligonucleotide, wherein the amplifying step comprises performing PCR using a pair of primers, selected from the group consisting of:
(i)
(SEQ ID NO: 64)
CCTTCCTCTTACTCTCTGCCTGC
and
(SEQ ID NO: 65)
GACAGCGGGCACACTTACCAG;
(ii)
(SEQ ID NO: 66)
CATTGTTAGGGCCGAGGCTAGA
and
(SEQ ID NO: 67)
ACGCACTTGTTTTACCTGTATGAGTC;
(iii)
(SEQ ID NO: 218)
GCCTCCCTTCCTCTTACTCTCT;
and
GCAGCCTGGGTAAGCACAC;
(iv)
(SEQ ID NO: 68)
GCAGATTCAACTTTCACGTATCAAACC
and
(SEQ ID NO: 69)
TGCAGAGCACGGGCTTTAATGT;
(v)
(SEQ ID NO: 70)
CCTCGCAGACATTAAAGCCCGT
and
(SEQ ID NO: 71)
CCACCATCACCACTGCTGCTG;
(vi)
(SEQ ID NO: 72)
CAGTGGTGATGGTGGTGAGGC
and
(SEQ ID NO: 73)
CCTAGCCCATCTGTGAGTCCAAC;
(vii)
(SEQ ID NO: 74)
TGTCCTCCCAAACCTCAGTAGC
and
(SEQ ID NO: 75)
AGCTTGGCCAGTTCCTTTCTCT;
(viii)
(SEQ ID NO: 76)
GTGTCTCGAGTATGTGCGGTCC
and
(SEQ ID NO: 77)
CATCGTGGGCTGGTGGAAGAA;
(ix)
(SEQ ID NO: 192)
CTTTCACGTATCAAACCACCC
and
(SEQ ID NO: 193)
CACGGGCTTTAATGTCTGC;
(x)
(SEQ ID NO: 194)
AAGATCCCAGATTCCCTACTG
and
(SEQ ID NO: 195)
GTGGGCTGGTGGAAGAAC;
(xi)
(SEQ ID NO: 196)
GGCGAGAGGTCACCACTG
and
(SEQ ID NO: 197)
CCATCACCACTGCTGCTG;
(xii)
(SEQ ID NO: 198)
AACTGAATGTGAGTCTGGCA
and
(SEQ ID NO: 199)
CACTAGAGACGGAATGGGAC;
(xiii)
(SEQ ID NO: 200)
CAGTGGTGATGGTGGTGAG
and
(SEQ ID NO: 201)
CCAACTGTAGCCCTCTGTAG;
(xiv)
(SEQ ID NO: 78)
TCCATTGCCTCTCCTTCTGTGC
and
(SEQ ID NO: 79)
TGCCAACTCCTTCATGCACCT;
(xv)
(SEQ ID NO: 80)
CCTAGGGGATGTTCCAGATGGC
and
(SEQ ID NO: 81)
AATGTGGGTTTGTTGCCATGAAAC;
(xvi)
(SEQ ID NO: 82)
TCTATCGTGTCCCCACAGGGAA
and
(SEQ ID NO: 83)
GGGGAAAGGTTGAACCCAAGGA;
(xvii)
(SEQ ID NO: 84)
CTTCACGCCGCCTTCCACCG
and
(SEQ ID NO: 85)
CTCCGGGCCAGTACCTTGAAA;
(xviii)
(SEQ ID NO: 204)
AGCAACGCCCATTTCACC
and
(SEQ ID NO: 205)
GCTCAGCTGCAAAGAATGTG;
(xix)
(SEQ ID NO: 206)
CAGGCAAGATGAGCGAGGC
and
(SEQ ID NO: 207)
GGGCCAGTACCTTGAAAGCG;
(xx)
(SEQ ID NO: 86)
TCCTGCTGTGTGGTTAGACGG
and
(SEQ ID NO: 87)
GAAGAGGTGGCGGATGACTGG;
(xxi)
(SEQ ID NO: 202)
CTGCTGTGTGGTTAGACGG
and
(SEQ ID NO: 203)
GAGGTGGCGGATGACTGG;
(xxii)
(SEQ ID NO: 88)
GCCCTGGGCATTCCTTCTTTATT
and
(SEQ ID NO: 89)
ACTTCTAAGATGTGGCAAGATGGC;
(xxiii)
(SEQ ID NO: 90)
GTTGGGGCATAGTTAAAACCTGTGT
and
(SEQ ID NO: 91)
TTCCTCTGTGTTGGCGGATACC;
(xxiv)
(SEQ ID NO: 92)
GGTATCCGCCAACACAGAGGAA
and
(SEQ ID NO: 93)
AGGAGACTGGAATTCTCGAATAAGGA;
(XXV)
(SEQ ID NO: 208)
GGCTGCTGGTCTGGCTACTAT
and
(SEQ ID NO: 209)
CCAGGGCAGAGGCTACAAC;
(xxvi)
(SEQ ID NO: 210)
TGTAGGTCTTGTGGATGAGC
and
(SEQ ID NO: 211)
TTCCTCTGTGTTGGCGGATA;
(xxvii)
(SEQ ID NO: 212)
CAGAGGAAAGGTAAATCCACCA
and
(SEQ ID NO: 213)
GGAATAAGATACCCAATAGCCTTCA;
(xxviii)
(SEQ ID NO: 214)
TTCCTTCTTTATTGCCCTTCTTA
and
(SEQ ID NO: 215)
CAACTTACCATGTTCAATGATTTCAA;
(xxix) a pair of primers having the reverse complement sequences of any of (i) through (xxviii); and
(xxx) a pair of primers having at least 90% sequence identity to any of (i) through (xxix);
(b) determining that the double-stranded oligonucleotide comprises a mutation in the gene of interest by means of a High Resolution Melting (HRM) analysis;
(c) sequencing the mutation;
(d) treating the subject in need of treatment of the anemia or cytopenia with an effective amount of a therapeutic agent.

115. The method of claim 114, wherein the determining step comprises comparing HRM analysis results of the double-stranded oligonucleotide to a control.

116. The method of claim 115, wherein the control comprises HRM analysis results of a wild-type sample.

117. The method of claim 115, wherein the control comprises HRM analysis results of a sample comprising the mutation of interest.

118. The method of any of claims 112-117, wherein the HRM analysis is performed using a dye.

119. The method of claim 118, wherein the dye comprises SYTO® 9, SYBR® Green, or Chai Green™.

120. The method of any one of claims 112-119, wherein the amplifying step comprises performing PCR using, in parallel, at least 2 pairs of primers from (i) through (xxx).

121. The method of any one of claims 112-119, wherein the amplifying step comprises performing PCR using, in parallel, at least 3 pairs of primers from (i) through (xxx).

122. The method of any one of claims 112-119, wherein the amplifying step comprises performing PCR using, in parallel, at least 4 pairs of primers from (i) through (xxx).

123. The method of any one of claims 112-122, wherein the gene of interest comprises WT1, and wherein the pair of primers are selected from the list consisting of:
(i)
(SEQ ID NO: 218)
GCCTCCCTTCCTCTTACTCTCT;
and
GCAGCCTGGGTAAGCACAC;
(ii) a pair of primers having the reverse complement sequences of (i); and
(iii) a pair of primers having at least 90% sequence identity to any of (i) through (ii). X. The method of any preceding claim, wherein the gene of interest comprises ASXL1, and wherein the pair of primers are selected from the list consisting of:
(i)
(SEQ ID NO: 192)
CTTTCACGTATCAAACCACCC;
and
(SEQ ID NO: 193)
CACGGGCTTTAATGTCTGC;
(ii)
(SEQ ID NO: 194)
AAGATCCCAGATTCCCTACTG;
and
(SEQ ID NO: 195)
GTGGGCTGGTGGAAGAAC;
(iii)
(SEQ ID NO: 196)
GGCGAGAGGTCACCACTG;
and
(SEQ ID NO: 197)
CCATCACCACTGCTGCTG;
(iv)
(SEQ ID NO: 198)
AACTGAATGTGAGTCTGGCA;
and
(SEQ ID NO: 199)
CACTAGAGACGGAATGGGAC;
(v)
(SEQ ID NO: 200)
CAGTGGTGATGGTGGTGAG;
and
(SEQ ID NO: 201)
CCAACTGTAGCCCTCTGTAG;
(vi) a pair of primers having the reverse complement sequences of any of (i) through (v); and
(vii) a pair of primers having at least 90% sequence identity to any of (i) through (vi).

124. The method of any one of claims 112-122, wherein the gene of interest comprises RUNX1, and wherein the pair of primers are selected from the list consisting of:
(i)
(SEQ ID NO: 204)
AGCAACGCCCATTTCACC;
and
(SEQ ID NO: 205)
GCTCAGCTGCAAAGAATGTG;
(ii)
(SEQ ID NO: 206)
CAGGCAAGATGAGCGAGGC;
and
(SEQ ID NO: 207)
GGGCCAGTACCTTGAAAGCG;
(iii) a pair of primers having the reverse complement sequences of any of (i) through (ii); and
(iv) a pair of primers having at least 90% sequence identity to any of (i) through (iii).

125. The method of any one of claims 112-122, wherein the gene of interest comprises DNMT3A, and wherein the pair of primers are selected from the list consisting of:
(i)
(SEQ ID NO: 202)
CTGCTGTGTGGTTAGACGG;
and
(SEQ ID NO: 203)
GAGGTGGCGGATGACTGG;
(ii) a pair of primers having the reverse complement sequences of (i); and
(iii) a pair of primers having at least 90% sequence identity to any of (i) through (ii).

126. The method of any one of claims 112-122, wherein the gene of interest comprises SFB31, and wherein the pair of primers are selected from the list consisting of:
(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;
(v) a pair of primers having the reverse complement sequences of any of (i) through (iv); and
(vi) a pair of primers having at least 90% sequence identity to any of (i) through (v).

127. The method of any one of claims 112-126, wherein at least one of the pair of primers comprises a tag.

128. The method of claim 127, wherein the tag comprises M13F.

129. A kit comprising:
(a) a pair of primers capable of amplifying a region of a gene of interest from a sample to produce a double-stranded oligonucleotide, wherein the pair of primers is selected from the group consisting of:
(i)
(SEQ ID NO: 64)
CCTTCCTCTTACTCTCTGCCTGC
and
(SEQ ID NO: 65)
GACAGCGGGCACACTTACCAG;
(ii)
(SEQ ID NO: 66)
CATTGTTAGGGCCGAGGCTAGA
and
(SEQ ID NO: 67)
ACGCACTTGTTTTACCTGTATGAGTC;
(iii)
(SEQ ID NO: 218)
GCCTCCCTTCCTCTTACTCTCT;
and
GCAGCCTGGGTAAGCACAC;
(iv)
(SEQ ID NO: 68)
GCAGATTCAACTTTCACGTATCAAACC
and
(SEQ ID NO: 69)
TGCAGAGCACGGGCTTTAATGT;
(v)
(SEQ ID NO: 70)
CCTCGCAGACATTAAAGCCCGT
and
(SEQ ID NO: 71)
CCACCATCACCACTGCTGCTG;
(vi)
(SEQ ID NO: 72)
CAGTGGTGATGGTGGTGAGGC
and
(SEQ ID NO: 73)
CCTAGCCCATCTGTGAGTCCAAC;
(vii)
(SEQ ID NO: 74)
TGTCCTCCCAAACCTCAGTAGC
and
(SEQ ID NO: 75)
AGCTTGGCCAGTTCCTTTCTCT;
(viii)
(SEQ ID NO: 76)
GTGTCTCGAGTATGTGCGGTCC
and
(SEQ ID NO: 77)
CATCGTGGGCTGGTGGAAGAA;
(ix)
(SEQ ID NO: 192)
CTTTCACGTATCAAACCACCC
and
(SEQ ID NO: 193)
CACGGGCTTTAATGTCTGC;
(x)
(SEQ ID NO: 194)
AAGATCCCAGATTCCCTACTG
and
(SEQ ID NO: 195)
GTGGGCTGGTGGAAGAAC;
(xi)
(SEQ ID NO: 196)
GGCGAGAGGTCACCACTG
and
(SEQ ID NO: 197)
CCATCACCACTGCTGCTG;
(xii)
(SEQ ID NO: 198)
AACTGAATGTGAGTCTGGCA
and
(SEQ ID NO: 199)
CACTAGAGACGGAATGGGAC;
(xiii)
(SEQ ID NO: 200)
CAGTGGTGATGGTGGTGAG
and
(SEQ ID NO: 201)
CCAACTGTAGCCCTCTGTAG;
(xiv)
(SEQ ID NO: 78)
TCCATTGCCTCTCCTTCTGTGC
and
(SEQ ID NO: 79)
TGCCAACTCCTTCATGCACCT;
(xv)
(SEQ ID NO: 80)
CCTAGGGGATGTTCCAGATGGC
and
(SEQ ID NO: 81)
AATGTGGGTTTGTTGCCATGAAAC;
(xvi)
(SEQ ID NO: 82)
TCTATCGTGTCCCCACAGGGAA
and
(SEQ ID NO: 83)
GGGGAAAGGTTGAACCCAAGGA;
(xvii)
(SEQ ID NO: 84)
CTTCACGCCGCCTTCCACCG
and
(SEQ ID NO: 85)
CTCCGGGCCAGTACCTTGAAA;
(xviii)
(SEQ ID NO: 204)
AGCAACGCCCATTTCACC
and
(SEQ ID NO: 205)
GCTCAGCTGCAAAGAATGTG;
(xix)
(SEQ ID NO: 206)
CAGGCAAGATGAGCGAGGC
and
(SEQ ID NO: 207)
GGGCCAGTACCTTGAAAGCG;
(xx)
(SEQ ID NO: 86)
TCCTGCTGTGTGGTTAGACGG
and
(SEQ ID NO: 87)
GAAGAGGTGGCGGATGACTGG;
(xxi)
(SEQ ID NO: 202)
CTGCTGTGTGGTTAGACGG
and
(SEQ ID NO: 203)
GAGGTGGCGGATGACTGG;
(xxii)
(SEQ ID NO: 88)
GCCCTGGGCATTCCTTCTTTATT
and
(SEQ ID NO: 89)
ACTTCTAAGATGTGGCAAGATGGC;
(xxiii)
(SEQ ID NO: 90)
GTTGGGGCATAGTTAAAACCTGTGT
and
(SEQ ID NO: 91)
TTCCTCTGTGTTGGCGGATACC;
(xxiv)
(SEQ ID NO: 92)
GGTATCCGCCAACACAGAGGAA
and
(SEQ ID NO: 93)
AGGAGACTGGAATTCTCGAATAAGGA;
(xxv)
(SEQ ID NO: 208)
GGCTGCTGGTCTGGCTACTAT
and
(SEQ ID NO: 209)
CCAGGGCAGAGGCTACAAC;
(xxvi)
(SEQ ID NO: 210)
TGTAGGTCTTGTGGATGAGC
and
(SEQ ID NO: 211)
TTCCTCTGTGTTGGCGGATA;
(xxvii)
(SEQ ID NO: 212)
CAGAGGAAAGGTAAATCCACCA
and
(SEQ ID NO: 213)
GGAATAAGATACCCAATAGCCTTCA;
(xxviii)
(SEQ ID NO: 214)
TTCCTTCTTTATTGCCCTTCTTA
and
(SEQ ID NO: 215)
CAACTTACCATGTTCAATGATTTCAA;
(xxix) a pair of primers having the reverse complement sequences of any of (i) through (xxviii); and
(xxx) a pair of primers having at least 90% sequence identity to any of (i) through (xxix);
(b) one or more reagents suitable for performing High Resolution Melting (HRM) analysis.

130. The kit of claim 129, wherein the kit comprises at least 2 pairs of primers from (i) through (xxx).

131. The kit of claim 129, wherein the kit comprises at least 3 pairs of primers from (i) through (xxx).

132. The kit of claim 129, wherein the kit comprises at least 4 pairs of primers from (i) through (xxx).

133. The kit of any of claims 129-132, further comprising a wild-type control.

134. The kit of any of claims 129-133, further comprising a mutant control.

135. The kit of any of claims 129-134, further comprising a dye.

136. The kit of claim 135, wherein the dye comprises SYTO® 9, SYBR® Green, or Chai Green™.

137. The kit of any preceding claim, wherein the gene of interest comprises WT1, and wherein the pair of primers are selected from the list consisting of:
(i)
(SEQ ID NO: 218)
GCCTCCCTTCCTCTTACTCTCT;
and
GCAGCCTGGGTAAGCACAC;
(ii) a pair of primers having the reverse complement sequences of (i); and
(iii) a pair of primers having at least 90% sequence identity to any of (i) through (ii). X. The method of any preceding claim, wherein the gene of interest comprises ASXL1, and wherein the pair of primers are selected from the list consisting of:
(i)
(SEQ ID NO: 192)
CTTTCACGTATCAAACCACCC
and
(SEQ ID NO: 193)
CACGGGCTTTAATGTCTGC;
(ii)
(SEQ ID NO: 194)
AAGATCCCAGATTCCCTACTG
and
(SEQ ID NO: 195)
GTGGGCTGGTGGAAGAAC;
(iii)
(SEQ ID NO: 196)
GGCGAGAGGTCACCACTG
and
(SEQ ID NO: 197)
CCATCACCACTGCTGCTG;
(iv)
(SEQ ID NO: 198)
AACTGAATGTGAGTCTGGCA
and
(SEQ ID NO: 199)
CACTAGAGACGGAATGGGAC;
(v)
(SEQ ID NO: 200)
CAGTGGTGATGGTGGTGAG
and
(SEQ ID NO: 201)
CCAACTGTAGCCCTCTGTAG;
(vi) a pair of primers having the reverse complement sequences of any of (i) through (v); and
(vii) a pair of primers having at least 90% sequence identity to any of (i) through (vi).

138. The kit of any one of claims 129-137, wherein the gene of interest comprises RUNX1, and wherein the pair of primers are selected from the list consisting of:
(ii)
(SEQ ID NO: 204)
AGCAACGCCCATTTCACC
and
(SEQ ID NO: 205)
GCTCAGCTGCAAAGAATGTG;
(ii)
(SEQ ID NO: 206)
CAGGCAAGATGAGCGAGGC
and
(SEQ ID NO: 207)
GGGCCAGTACCTTGAAAGCG;
(iii) a pair of primers having the reverse complement sequences of any of (i) through (ii); and
(iv) a pair of primers having at least 90% sequence identity to any of (i) through (iii).

139. The kit of any one of claims 129-137, wherein the gene of interest comprises DNMT3A, and wherein the pair of primers are selected from the list consisting of:
(i)
(SEQ ID NO: 202)
CTGCTGTGTGGTTAGACGG
and
(SEQ ID NO: 203)
GAGGTGGCGGATGACTGG;
(ii) a pair of primers having the reverse complement sequences of (i); and
(iii) a pair of primers having at least 90% sequence identity to any of (i) through (ii).

140. The kit of any one of claims 129-137, wherein the gene of interest comprises SFB31, and wherein the pair of primers are selected from the list consisting of:
(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;
(v) a pair of primers having the reverse complement sequences of any of (i) through (iv); and
(vi) a pair of primers having at least 90% sequence identity to any of (i) through (v).

141. The kit of any one of claims 129-140, wherein at least one of the pair of primers comprises a tag.

142. The kit of claim 141, wherein the tag comprises M13F.

143. A method for determining the presence or absence of a mutation in a gene of interest, the method comprising:
(a) amplifying a region of a gene of interest from a sample to produce a double-stranded oligonucleotide, wherein the amplifying step comprises performing PCR using a pair of primers, selected from the group consisting of:
(i)
(SEQ ID NO: 88)
GCCCTGGGCATTCCTTCTTTATT
and
(SEQ ID NO: 89)
ACTTCTAAGATGTGGCAAGATGGC;
(ii)
(SEQ ID NO: 90)
GTTGGGGCATAGTTAAAACCTGTGT
and
(SEQ ID NO: 91)
TTCCTCTGTGTTGGCGGATACC;
(iii)
(SEQ ID NO: 92)
GGTATCCGCCAACACAGAGGAA
and
(SEQ ID NO: 93)
AGGAGACTGGAATTCTCGAATAAGGA;
(iv)
(SEQ ID NO: 208)
GGCTGCTGGTCTGGCTACTAT
and
(SEQ ID NO: 209)
CCAGGGCAGAGGCTACAAC;
(v)
(SEQ ID NO: 210)
TGTAGGTCTTGTGGATGAGC
and
(SEQ ID NO: 211)
TTCCTCTGTGTTGGCGGATA;
(vi)
(SEQ ID NO: 212)
CAGAGGAAAGGTAAATCCACCA
and
(SEQ ID NO: 213)
GGAATAAGATACCCAATAGCCTTCA;
(vii)
(SEQ ID NO: 214)
TTCCTTCTTTATTGCCCTTCTTA
and
(SEQ ID NO: 215)
CAACTTACCATGTTCAATGATTTCAA;
(viii)
(SEQ ID NO: 230)
CTACTCCTCGCCTGTGGACAAC
and
(SEQ ID NO: 231)
GATCTGGGACTGCATGCTGGTG;
(ix)
(SEQ ID NO: 220)
CCAACGTCAGTGAGGCAGAT
and
(SEQ ID NO: 221)
GGATGACAATACCAGGCAGGA;
(x)
(SEQ ID NO: 222)
TGCTTTCCTTGGAGCCAAAT
and
(SEQ ID NO: 223)
TGTGTTAGCTGGAGTGAAAACTTG;
(xi)
(SEQ ID NO: 224)
ATATGCCTGAGCGTTTCGATGC
and
(SEQ ID NO: 225)
GTGGCCTTCTAGCCAACCTCT;
(xii)
(SEQ ID NO: 226)
CCCAGGGGATATGCTGAACTA
and
(SEQ ID NO: 227)
ACCTGGAGAGAGGCTGAGTG;
(xiii)
(SEQ ID NO: 228)
TTGGCTTGCAGGTGCCCATC
and
(SEQ ID NO: 229)
GGCGAGTCCAGAACCAAGATTT;
(xiv) a pair of primers having the reverse complement sequences of any of (i) through (xiii); and
(xv) a pair of primers having at least 90% sequence identity to any of (i) through (xiv);
(b) determining that the double-stranded oligonucleotide comprises or does not comprise the mutation in the gene of interest by means of a High Resolution Melting (HRM) analysis.

144. The method of claim 143, further comprising the step of sequencing the mutation.

145. The method of claim 143 or claim 144, wherein the sample is from a subject in need of treatment of chronic lymphocytic leukemia, and the method further comprises treating the subject with an effective amount of a therapeutic agent to treat the chronic lymphocytic leukemia.

146. The method of any one of claims 143-145, wherein the determining step comprises comparing HRM analysis results of the double-stranded oligonucleotide to a control.

147. The method of claim 146, wherein the control comprises HRM analysis results of a wild-type sample.

148. The method of claim 146, wherein the control comprises HRM analysis results of a sample comprising the mutation of interest.

149. The method of any one of claims 146-148, wherein the HRM analysis is performed using a dye.

150. The method of claim 149, wherein the dye comprises SYTO® 9, SYBR® Green, or Chai Green™.

151. The method of any one of claims 143-150, wherein the amplifying step comprises performing PCR using, in parallel, at least 2 pairs of primers from (i) through (xiv).

152. The method of any one of claims 143-151, wherein the amplifying step comprises performing PCR using, in parallel, at least 3 pairs of primers from (i) through (iv).

153. The method of any one of claims 143-152, wherein the amplifying step comprises performing PCR using, in parallel, at least 4 pairs of primers from (i) through (iv).

154. The method of any one of claims 143-153, wherein the gene of interest comprises SFB31, and wherein the pair of primers are selected from the list consisting of:
(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;
(v) a pair of primers having the reverse complement sequences of any of (i) through (iv); and
(vi) a pair of primers having at least 90% sequence identity to any of (i) through (v).

155. The method of any one of claims 143-153, wherein the gene of interest comprises NOTCH1, and wherein the pair of primers are selected from the list consisting of:
(i)
(SEQ ID NO: 230)
CTACTCCTCGCCTGTGGACAAC
and
(SEQ ID NO: 231)
GATCTGGGACTGCATGCTGGTG;
(ii) a pair of primers having the reverse complement sequences of (i); and
(iii) a pair of primers having at least 90% sequence identity to any of (i) through (ii).

156. The method of any one of claims 143-153, wherein the gene of interest comprises CXCR4, and wherein the pair of primers are selected from the list consisting of:
(i)
(SEQ ID NO: 220)
CCAACGTCAGTGAGGCAGAT
and
(SEQ ID NO: 221)
GGATGACAATACCAGGCAGGA;
(ii)
(SEQ ID NO: 222)
TGCTTTCCTTGGAGCCAAAT
and
(SEQ ID NO: 223)
TGTGTTAGCTGGAGTGAAAACTTG;
(iii) a pair of primers having the reverse complement sequences of any of (i) through (ii); and
(iv) a pair of primers having at least 90% sequence identity to any of (i) through (iii).

157. The method of any one of claims 143-153, wherein the gene of interest comprises MYD88, and wherein the pair of primers are selected from the list consisting of:
(x)
(SEQ ID NO: 224)
ATATGCCTGAGCGTTTCGATGC
and
(SEQ ID NO: 225)
GTGGCCTTCTAGCCAACCTCT;
(ii)
(SEQ ID NO: 226)
CCCAGGGGATATGCTGAACTA
and
(SEQ ID NO: 227)
ACCTGGAGAGAGGCTGAGTG;
(iii)
(SEQ ID NO: 228)
TTGGCTTGCAGGTGCCCATC
and
(SEQ ID NO: 229)
GGCGAGTCCAGAACCAAGATTT;
(iv) a pair of primers having the reverse complement sequences of any of (i) through (iii); and
(v) a pair of primers having at least 90% sequence identity to any of (i) through (iv).

158. The method of any one of claims 143-157, wherein at least one of the pair of primers comprises a tag.

159. The method of claim 158, wherein the tag comprises M13F.

160. A method of treating a subject in need of treatment of chronic lymphocytic leukemia, the method comprising:
(a) amplifying a region of a gene of interest from a sample from the subject to produce a double-stranded oligonucleotide, wherein the amplifying step comprises performing PCR using a pair of primers, selected from the group consisting of:
(i)
(SEQ ID NO: 88)
GCCCTGGGCATTCCTTCTTTATT
and
(SEQ ID NO: 89)
ACTTCTAAGATGTGGCAAGATGGC;
(ii)
(SEQ ID NO: 90)
GTTGGGGCATAGTTAAAACCTGTGT
and
(SEQ ID NO: 91)
TTCCTCTGTGTTGGCGGATACC;
(iii)
(SEQ ID NO: 92)
GGTATCCGCCAACACAGAGGAA
and
(SEQ ID NO: 93)
AGGAGACTGGAATTCTCGAATAAGGA;
(iv)
(SEQ ID NO: 208)
GGCTGCTGGTCTGGCTACTAT
and
(SEQ ID NO: 209)
CCAGGGCAGAGGCTACAAC;
(v)
(SEQ ID NO: 210)
TGTAGGTCTTGTGGATGAGC
and
(SEQ ID NO: 211)
TTCCTCTGTGTTGGCGGATA;
(vi)
(SEQ ID NO: 212)
CAGAGGAAAGGTAAATCCACCA
and
(SEQ ID NO: 213)
GGAATAAGATACCCAATAGCCTTCA;
(vii)
(SEQ ID NO: 214)
TTCCTTCTTTATTGCCCTTCTTA
and
(SEQ ID NO: 215)
CAACTTACCATGTTCAATGATTTCAA;
(viii)
(SEQ ID NO: 230)
CTACTCCTCGCCTGTGGACAAC
and
(SEQ ID NO: 231)
GATCTGGGACTGCATGCTGGTG;
(ix)
(SEQ ID NO: 220)
CCAACGTCAGTGAGGCAGAT
and
(SEQ ID NO: 221)
GGATGACAATACCAGGCAGGA;
(x)
(SEQ ID NO: 222)
TGCTTTCCTTGGAGCCAAAT
and
(SEQ ID NO: 223)
TGTGTTAGCTGGAGTGAAAACTTG;
(xi)
(SEQ ID NO: 224)
ATATGCCTGAGCGTTTCGATGC
and
(SEQ ID NO: 225)
GTGGCCTTCTAGCCAACCTCT;
(xii)
(SEQ ID NO: 226)
CCCAGGGGATATGCTGAACTA
and
(SEQ ID NO: 227)
ACCTGGAGAGAGGCTGAGTG;
(xiii)
(SEQ ID NO: 228)
TTGGCTTGCAGGTGCCCATC
and
(SEQ ID NO: 229)
GGCGAGTCCAGAACCAAGATTT;
(xiv) a pair of primers having the reverse complement sequences of any of (i) through (xiii); and
(xv) a pair of primers having at least 90% sequence identity to any of (i) through (xiv);
(b) determining that the double-stranded oligonucleotide comprises a mutation in the gene of interest by means of a High Resolution Melting (HRM) analysis;
(c) sequencing the mutation;
(d) treating the subject in need of treatment of the chronic lymphocytic leukemia with an effective amount of a therapeutic agent.

161. The method of claim 160, wherein the determining step comprises comparing HRM analysis results of the double-stranded oligonucleotide to a control.

162. The method of claim 161, wherein the control comprises HRM analysis results of a wild-type sample.

163. The method of claim 161, wherein the control comprises HRM analysis results of a sample comprising the mutation of interest.

164. The method of any of claims 160-163, wherein the HRM analysis is performed using a dye.

165. The method of claim 164, wherein the dye comprises SYTO® 9, SYBR® Green, or Chai Green™.

166. The method of any one of claims 160-165, wherein the amplifying step comprises performing PCR using, in parallel, at least 2 pairs of primers from (i) through (xiv).

167. The method of any one of claims 160-165, wherein the amplifying step comprises performing PCR using, in parallel, at least 3 pairs of primers from (i) through (xiv).

168. The method of any one of claims 160-165, wherein the amplifying step comprises performing PCR using, in parallel, at least 4 pairs of primers from (i) through (xiv).

169. The method of any one of claims 160-168, wherein the gene of interest comprises SFB31, and wherein the pair of primers are selected from the list consisting of:
(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;
(v) a pair of primers having the reverse complement sequences of any of (i) through (iv); and
(vi) a pair of primers having at least 90% sequence identity to any of (i) through (v).

170. The method of any one of claims 160-168, wherein the gene of interest comprises NOTCH1, and wherein the pair of primers are selected from the list consisting of:
(i)
(SEQ ID NO: 230)
CTACTCCTCGCCTGTGGACAAC
and
(SEQ ID NO: 231)
GATCTGGGACTGCATGCTGGTG;
(ii) a pair of primers having the reverse complement sequences of (i); and
(iii) a pair of primers having at least 90% sequence identity to any of (i) through (ii).

171. The method of any one of claims 160-168, wherein the gene of interest comprises CXCR4, and wherein the pair of primers are selected from the list consisting of:
(i)
(SEQ ID NO: 220)
CCAACGTCAGTGAGGCAGAT
and
(SEQ ID NO: 221)
GGATGACAATACCAGGCAGGA;
(ii)
(SEQ ID NO: 222)
TGCTTTCCTTGGAGCCAAAT
and
(SEQ ID NO: 223)
TGTGTTAGCTGGAGTGAAAACTTG;
(iii) a pair of primers having the reverse complement sequences of any of (i) through (ii); and
(iv) a pair of primers having at least 90% sequence identity to any of (i) through (iii).

172. The method of any one of claims 160-168, wherein the gene of interest comprises MYD88, and wherein the pair of primers are selected from the list consisting of:
(i)
(SEQ ID NO: 224)
ATATGCCTGAGCGTTTCGATGC
and
(SEQ ID NO: 225)
GTGGCCTTCTAGCCAACCTCT;
(ii)
(SEQ ID NO: 226)
CCCAGGGGATATGCTGAACTA
and
(SEQ ID NO: 227)
ACCTGGAGAGAGGCTGAGTG;
(iii)
(SEQ ID NO: 228)
TTGGCTTGCAGGTGCCCATC
and
(SEQ ID NO: 229)
GGCGAGTCCAGAACCAAGATTT;
(iv) a pair of primers having the reverse complement sequences of any of (i) through (iii); and
(v) a pair of primers having at least 90% sequence identity to any of (i) through (iv).

173. The method of any one of claims 160-172, wherein at least one of the pair of primers comprises a tag.

174. The method of claim 173, wherein the tag comprises M13F.

175. A kit comprising:
(a) a pair of primers capable of amplifying a region of a gene of interest from a sample to produce a double-stranded oligonucleotide, wherein the pair of primers is selected from the group consisting of:
(i)
(SEQ ID NO: 88)
GCCCTGGGCATTCCTTCTTTATT
and
(SEQ ID NO: 89)
ACTTCTAAGATGTGGCAAGATGGC;
(ii)
(SEQ ID NO: 90)
GTTGGGGCATAGTTAAAACCTGTGT
and
(SEQ ID NO: 91)
TTCCTCTGTGTTGGCGGATACC;
(iii)
(SEQ ID NO: 92)
GGTATCCGCCAACACAGAGGAA
and
(SEQ ID NO: 93)
AGGAGACTGGAATTCTCGAATAAGGA;
(iv)
(SEQ ID NO: 208)
GGCTGCTGGTCTGGCTACTAT
and
(SEQ ID NO: 209)
CCAGGGCAGAGGCTACAAC;
(v)
(SEQ ID NO: 210)
TGTAGGTCTTGTGGATGAGC
and
(SEQ ID NO: 211)
TTCCTCTGTGTTGGCGGATA;
(vi)
(SEQ ID NO: 212)
CAGAGGAAAGGTAAATCCACCA
and
(SEQ ID NO: 213)
GGAATAAGATACCCAATAGCCTTCA;
(vii)
(SEQ ID NO: 214)
TTCCTTCTTTATTGCCCTTCTTA
and
(SEQ ID NO: 215)
CAACTTACCATGTTCAATGATTTCAA;
(viii)
(SEQ ID NO: 230)
CTACTCCTCGCCTGTGGACAAC
and
(SEQ ID NO: 231)
GATCTGGGACTGCATGCTGGTG;
(ix)
(SEQ ID NO: 220)
CCAACGTCAGTGAGGCAGAT
and
(SEQ ID NO: 221)
GGATGACAATACCAGGCAGGA;
(x)
(SEQ ID NO: 222)
TGCTTTCCTTGGAGCCAAAT
and
(SEQ ID NO: 223)
TGTGTTAGCTGGAGTGAAAACTTG;
(xi)
(SEQ ID NO: 224)
ATATGCCTGAGCGTTTCGATGC
and
(SEQ ID NO: 225)
GTGGCCTTCTAGCCAACCTCT;
(xii)
(SEQ ID NO: 226)
CCCAGGGGATATGCTGAACTA
and
(SEQ ID NO: 227)
ACCTGGAGAGAGGCTGAGTG;
(xiii)
(SEQ ID NO: 228)
TTGGCTTGCAGGTGCCCATC
and
(SEQ ID NO: 229)
GGCGAGTCCAGAACCAAGATTT;
(xiv) a pair of primers having the reverse complement sequences of any of (i) through (xiii); and
(xv) a pair of primers having at least 90% sequence identity to any of (i) through (xiv);
(b) one or more reagents suitable for performing High Resolution Melting (HRM) analysis.

176. The kit of claim 175, wherein the kit comprises at least 2 pairs of primers from (i) through (xiv).

177. The kit of claim 175, wherein the kit comprises at least 3 pairs of primers from (i) through (xiv).

178. The kit of claim 175, wherein the kit comprises at least 4 pairs of primers from (i) through (xiv).

179. The kit of any of claims 175-178, further comprising a wild-type control.

180. The kit of any of claims 175-179, further comprising a mutant control.

181. The kit of any of claims 175-180, further comprising a dye.

182. The kit of claim 181, wherein the dye comprises SYTO® 9, SYBR® Green, or Chai Green™.

183. The kit of any one of claims 175-182, wherein the gene of interest comprises SFB31, and wherein the pair of primers are selected from the list consisting of:
(i)
(SEQ ID NO: 208)
GGCTGCTGGTCTGGCTACTAT
and
(SEQ ID NO: 209)
CCAGGGCAGAGGCTACAAC;
(ii)
(SEQ ID NO: 210)
TGTAGGTCTTGTGGATGAGC
and
(SEQ ID NO: 211)
TTCCTCTGTGTTGGCGGATA;
(iii)
(SEQ ID NO: 212)
CAGAGGAAAGGTAAATCCACCA
and
(SEQ ID NO: 213)
GGAATAAGATACCCAATAGCCTTCA;
(iv)
(SEQ ID NO: 214)
TTCCTTCTTTATTGCCCTTCTTA
and
(SEQ ID NO: 215)
CAACTTACCATGTTCAATGATTTCAA;
(v) a pair of primers having the reverse complement sequences of any of (i) through (iv); and
(vi) a pair of primers having at least 90% sequence identity to any of (i) through (v).

184. The kit of any one of claims 175-182, wherein the gene of interest comprises NOTCH1, and wherein the pair of primers are selected from the list consisting of:
(i)
(SEQ ID NO: 230)
CTACTCCTCGCCTGTGGACAAC
and
(SEQ ID NO: 231)
GATCTGGGACTGCATGCTGGTG;
(ii) a pair of primers having the reverse complement sequences of (i); and
(iii) a pair of primers having at least 90% sequence identity to any of (i) through (ii).

185. The kit of any one of claims 175-182, wherein the gene of interest comprises CXCR4, and wherein the pair of primers are selected from the list consisting of:
(i)
(SEQ ID NO: 220)
CCAACGTCAGTGAGGCAGAT
and
(SEQ ID NO: 221)
GGATGACAATACCAGGCAGGA;
(ii)
(SEQ ID NO: 222)
TGCTTTCCTTGGAGCCAAAT
and
(SEQ ID NO: 223)
TGTGTTAGCTGGAGTGAAAACTTG;
(iii) a pair of primers having the reverse complement sequences of any of (i) through (ii); and
(iv) a pair of primers having at least 90% sequence identity to any of (i) through (iii).

186. The kit of any one of claims 175-182, wherein the gene of interest comprises MYD88, and wherein the pair of primers are selected from the list consisting of:
(i)
(SEQ ID NO: 224)
ATATGCCTGAGCGTTTCGATGC
and
(SEQ ID NO: 225)
GTGGCCTTCTAGCCAACCTCT;
(ii)
(SEQ ID NO: 226)
CCCAGGGGATATGCTGAACTA
and
(SEQ ID NO: 227)
ACCTGGAGAGAGGCTGAGTG;
(iii)
(SEQ ID NO: 228)
TTGGCTTGCAGGTGCCCATC
and
(SEQ ID NO: 229)
GGCGAGTCCAGAACCAAGATTT;
(iv) a pair of primers having the reverse complement sequences of any of (i) through (iii); and
(v) a pair of primers having at least 90% sequence identity to any of (i) through (iv).

187. The of any one of claims 175-186, wherein at least one of the pair of primers comprises a tag.

188. The kit of claim 187, wherein the tag comprises M13F.

189. A method of treating a subject in need of treatment of BCR/ABL1-induced leukemogenesis, the method comprising:
(a) amplifying a region of a gene of interest from a sample from the subject to produce a double-stranded oligonucleotide, wherein the amplifying step comprises performing PCR using two or more primers, selected from the group consisting of:
(i)
(SEQ ID NO: 243)
ATGCTGACCAACTCGTGTGTGAA;
(ii)
(SEQ ID NO: 244)
CAGAACTCGCAACAGTCCTTCGA;
(iii)
(SEQ ID NO: 245)
GTGCGTGGAGGAGATCGAGCG;
(iv)
(SEQ ID NO: 246)
TGATTATAGCCTAAGACCCGGAGC;
(v)
(SEQ ID NO: 247)
CCTAAGACCCGGAGCTTTTCAC;
(vi) two or more primers having the reverse complement sequences of any of (i) through (v); and
(vii) two or more primers having at least 90% sequence identity to any of (i) through (vi). (b) determining that the double-stranded oligonucleotide comprises an exon in the gene of interest by means of a High Resolution Melting (HRM) analysis;
(c) treating the subject in need of treatment of the chronic lymphocytic leukemia with an effective amount of a therapeutic agent.

190. The method of claim 189, wherein the determining step comprises comparing HRM analysis results of the double-stranded oligonucleotide to a control.

191. The method of claim 190, wherein the control comprises HRM analysis results of a wild-type sample.

192. The method of claim 190, wherein the control comprises HRM analysis results of a sample comprising the exon of interest.

193. The method of any of claims 189-192, wherein the HRM analysis is performed using a dye.

194. The method of claim 193, wherein the dye comprises SYTO® 9, SYBR® Green, or Chai Green™.

195. The method of any one of claims 189-194, wherein the amplifying step comprises performing PCR using, in parallel, at least 2 primers from (i) through (vii).

196. The method of any one of claims 189-194, wherein the amplifying step comprises performing PCR using, in parallel, at least 3 primers from (i) through (vii).

197. The method of any one of claims 189-194, wherein the amplifying step comprises performing PCR using, in parallel, at least 4 primers from (i) through (vii).

198. The method of any one of claims 189-197, wherein the gene of interest comprises BCR/ABL1 fusion gene, and wherein the primers are selected from the list consisting of:
(i)
(SEQ ID NO: 243)
ATGCTGACCAACTCGTGTGTGAA;
(iii)
(SEQ ID NO: 246)
TGATTATAGCCTAAGACCCGGAGC;
(iii)
(SEQ ID NO: 247)
CCTAAGACCCGGAGCTTTTCAC;
(iv) two or more primers having the reverse complement sequences of any of (i) through (iii); and
(v) two or more primers having at least 90% sequence identity to any of (i) through (iv).

199. The method of any one of claims 189-197, wherein the gene of interest comprises BCR/ABL1 fusion gene, and wherein the primers are selected from the list consisting of:
(i)
(SEQ ID NO: 244)
CAGAACTCGCAACAGTCCTTCGA;
(iii)
(SEQ ID NO: 246)
TGATTATAGCCTAAGACCCGGAGC;
(iii)
(SEQ ID NO: 247)
CCTAAGACCCGGAGCTTTTCAC;
(iv) two or more primers having the reverse complement sequences of any of (i) through (iii); and
(v) two or more primers having at least 90% sequence identity to any of (i) through (iv).

200. The method of any one of claims 189-197, wherein the gene of interest comprises BCR/ABL1 fusion gene, and wherein the primers are selected from the list consisting of:
(i)
(SEQ ID NO: 245)
GTGCGTGGAGGAGATCGAGCG;
(ii)
(SEQ ID NO: 246)
TGATTATAGCCTAAGACCCGGAGC;
(iii)
(SEQ ID NO: 247)
CCTAAGACCCGGAGCTTTTCAC;
(iv) two or more primers having the reverse complement sequences of any of (i) through (iii); and
(v) two or more primers having at least 90% sequence identity to any of (i) through (iv).

201. The method of any one of claims 189-200, wherein at least one primer comprises a tag.

202. The method of claim 201, wherein the tag comprises M13F.

203. A kit comprising:
(a) two or more primers capable of amplifying a region of a gene of interest from a sample to produce a double-stranded oligonucleotide, wherein the primers are selected from the group consisting of:
(i)
(SEQ ID NO: 243)
ATGCTGACCAACTCGTGTGTGAA;
(ii)
CAGAACTCGCAACAGTCCTTCGA;
(SEQ ID NO: 244)
(iii)
(SEQ ID NO: 245)
GTGCGTGGAGGAGATCGAGCG;
(iv)
(SEQ ID NO: 246)
TGATTATAGCCTAAGACCCGGAGC;
(v)
(SEQ ID NO: 247)
CCTAAGACCCGGAGCTTTTCAC;
(vi) two or more primers having the reverse complement sequences of any of (i) through (v); and
(vii) two or more primers having at least 90% sequence identity to any of (i) through (vi);
(b) two or more reagents suitable for performing High Resolution Melting (HRM) analysis.

204. The kit of claim 203, wherein the kit comprises at least 2 primers from (i) through (vii).

205. The kit of claim 203, wherein the kit comprises at least 3 primers from (i) through (vii).

206. The kit of claim 203, wherein the kit comprises at least 4 primers from (i) through (vii).

207. The kit of any of claims 203-206, further comprising a wild-type control.

208. The kit of any of claims 203-207, further comprising a mutant control.

209. The kit of any of claims 203-208, further comprising a dye.

210. The kit of claim 209, wherein the dye comprises SYTO® 9, SYBR® Green, or Chai Green™.

211. The kit of any one of claims 203-210, wherein the gene of interest comprises BCR/ABL1 fusion gene, and wherein the primers are selected from the list consisting of:
(i)
(SEQ ID NO: 243)
ATGCTGACCAACTCGTGTGTGAA;
(iii)
(SEQ ID NO: 246)
TGATTATAGCCTAAGACCCGGAGC;
(iii)
(SEQ ID NO: 247)
CCTAAGACCCGGAGCTTTTCAC;
(iv) two or more primers having the reverse complement sequences of any of (i) through (iii); and
(v) two or more primers having at least 90% sequence identity to any of (i) through (iv).

212. The kit of any one of claims 203-210, wherein the gene of interest comprises BCR/ABL1 fusion gene, and wherein the primers are selected from the list consisting of:
(i)
(SEQ ID NO: 244)
CAGAACTCGCAACAGTCCTTCGA;
(ii)
(SEQ ID NO: 246)
TGATTATAGCCTAAGACCCGGAGC;
(iii)
(SEQ ID NO: 247)
CCTAAGACCCGGAGCTTTTCAC;
(iv) two or more primers having the reverse complement sequences of any of (i) through (iii); and
(v) two or more primers having at least 90% sequence identity to any of (i) through (iv).

213. The kit of any one of claims 203-210, wherein the gene of interest comprises BCR/ABL1 fusion gene, and wherein the primers are selected from the list consisting of:
(i)
(SEQ ID NO: 245)
GTGCGTGGAGGAGATCGAGCG;
(ii)
(SEQ ID NO: 246)
TGATTATAGCCTAAGACCCGGAGC;
(iii)
(SEQ ID NO: 247)
CCTAAGACCCGGAGCTTTTCAC;
(iv) two or more primers having the reverse complement sequences of any of (i) through (iii); and
(v) two or more primers having at least 90% sequence identity to any of (i) through (iv).

214. The of any one of claims 203-213, wherein at least one of the primers comprises a tag.

215. The kit of claim 214, wherein the tag comprises M13F.

216. A method for performing a high resolution melting analysis (HRM) to detect the presence or absence of a mutation in a nucleic acid, the method comprising:
(a) combining the nucleic acid, at least one primer pair, a dye, and a reaction mixture comprising 10 mM Tris-HCl, 50 mM KCl, 1.5 mM MgCl₂, 0.2 mM dNTPs, 5% glycerol, and 25 units/mL of a DNA polymerase;
(b) amplifying the nucleic acid; and
(c) detecting the presence or absence of the mutation by performing a melting curve analysis of the amplified product.

217. The method of claim 216, wherein the DNA polymerase comprises Hot Start Taq DNA polymerase.

218. The method of claim 216 or 217, further comprising the step of sequencing the mutation.

219. The method of any one of claims 216-218, wherein the detecting step comprises comparing the HRM analysis results of the nucleic acid to a control.

220. The method of claim 219, wherein the control comprises HRM analysis results of a wild-type sample.

221. The method of claim 219, wherein the control comprises HRM analysis results of a sample comprising the mutation.

223. The method of any one of claims 216-221, wherein the dye comprises SYTO® 9, SYBR® Green, or Chai Green™.

224. A method for performing a high resolution melting analysis (HRM) to detect the presence or absence of a mutation in a nucleic acid, the method comprising:
(a) combining the nucleic acid, at least one primer pair, a DNA polymerase, dNTPs, optionally dUTPs, and a dye;
(b) amplifying the nucleic acid; and
(c) detecting the presence or absence of the mutation by performing a melting curve analysis of the amplified product.

225. The method of claim 224, wherein the DNA polymerase comprises AmpliTaq Gold™ 360 DNA Polymerase.

226. The method of claim 223 or 224, further comprising the step of sequencing the mutation.

227. The method of any one of claims 223-226, wherein the detecting step comprises comparing the HRM analysis results of the nucleic acid to a control.

228. The method of claim 227, wherein the control comprises HRM analysis results of a wild-type sample.

229. The method of claim 228, wherein the control comprises HRM analysis results of a sample comprising the mutation.

230. The method of any one of claims 223-229, wherein the dye comprises SYTO® 9, SYBR® Green, or Chai Green™.

231. The method of any one of claims 223-229, wherein the dye comprises MeltDoctor™ HRM Dye.
